SEQUENCE LISTING (126 sequences)

SEQ ID NO: 1 - DNA, 729 bp, synthetic construct
  source 1..729: mol_type = other DNA
gaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaa

SEQ ID NO: 2 - protein, 243 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 3 - DNA, 1473 bp, synthetic construct
  source 1..1473: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 4 - protein, 491 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 66 bp, synthetic construct
  source 1..66: mol_type = other DNA
atgctgctgctggtgaccagcctgctgctgtgcgaactgccgcatccggcgtttctgctgattccg

SEQ ID NO: 6 - protein, 22 aa, synthetic construct
MLLLVTSLLLCELPHPAFLLIP

SEQ ID NO: 7 - DNA, 747 bp, synthetic construct
  source 1..747: mol_type = other DNA
caggtacagctccagcagagtggcccagggctcgtgaagccaagccagacgctgtccctgacttgtgcaatttcaggggattcagtttcatcaaatagcgcggcgtggaattggattcgacaatctccttcccgagggttggaatggcttggacgaacatattacagatccaaatggtataacgactatgcggtatcagtaaagtcaagaataaccattaaccccgacacaagcaagaaccaattctctttgcagcttaactctgtcacgccagaagacacggcagtctattattgcgctcgcgaggtaacgggtgacctggaagacgcttttgacatttgggggcagggtacgatggtgacagtcagttcagggggcggtgggagtgggggagggggtagcggggggggagggtcagacattcagatgacccagtccccttcatccttgtctgcctccgtcggtgacagggtgacaataacatgcagagcaagccaaacaatctggagctatctcaactggtaccagcagcgaccaggaaaagcgccaaacctgctgatttacgctgcttcctccctccaatcaggcgtgcctagtagatttagcggtaggggctccggcaccgattttacgctcactataagctctcttcaagcagaagattttgcgacttattactgccagcagtcctatagtatacctcagactttcggacagggtaccaagttggagattaaggcggccgca

SEQ ID NO: 8 - protein, 249 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAA

SEQ ID NO: 9 - DNA, 1482 bp, synthetic construct
  source 1..1482: mol_type = other DNA
atgcttcttttggtgacttcccttttgctgtgcgagttgccacaccccgccttcctgcttattccccaggtacagctccagcagagtggcccagggctcgtgaagccaagccagacgctgtccctgacttgtgcaatttcaggggattcagtttcatcaaatagcgcggcgtggaattggattcgacaatctccttcccgagggttggaatggcttggacgaacatattacagatccaaatggtataacgactatgcggtatcagtaaagtcaagaataaccattaaccccgacacaagcaagaaccaattctctttgcagcttaactctgtcacgccagaagacacggcagtctattattgcgctcgcgaggtaacgggtgacctggaagacgcttttgacatttgggggcagggtacgatggtgacagtcagttcagggggcggtgggagtgggggagggggtagcggggggggagggtcagacattcagatgacccagtccccttcatccttgtctgcctccgtcggtgacagggtgacaataacatgcagagcaagccaaacaatctggagctatctcaactggtaccagcagcgaccaggaaaagcgccaaacctgctgatttacgctgcttcctccctccaatcaggcgtgcctagtagatttagcggtaggggctccggcaccgattttacgctcactataagctctcttcaagcagaagattttgcgacttattactgccagcagtcctatagtatacctcagactttcggacagggtaccaagttggagattaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 10 - protein, 494 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 12 - protein, 22 aa, synthetic construct
IWAPLAGTCGVLLLSLVITLYC

SEQ ID NO: 13 - DNA, 141 bp, synthetic construct
  source 1..141: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggactttgcctgcgatatctac

SEQ ID NO: 14 - protein, 47 aa, synthetic construct
TTTPAPRPPTPAPTIASQPLSLRPEACRPAAGGAVHTRGLDFACDIY

SEQ ID NO: 15 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 16 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 18 - protein, 42 aa, synthetic construct
KRGRKKLLYIFKQPFMRPVQTTQEEDGCSCRFPEEEEGGCEL

SEQ ID NO: 19 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1467 bp, synthetic construct
  source 1..1467: mol_type = other DNA
atgctgctgctggtgaccagcctgctgctgtgcgaactgccgcatccggcgtttctgctgattccggatattcagatgacccagaccaccagcagcctgagcgcgagcctgggcgatcgcgtgaccattagctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattatagcctgaccattagcaacctggaacaggaagatattgcgacctatttttgccagcagggcaacaccctgccgtatacctttggcggcggcaccaaactggaaattaccggcggcggcggcagcggcggcggcggcagcggcggcggcggcagcgaagtgaaactgcaggaaagcggcccgggcctggtggcgccgagccagagcctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattattaaagataacagcaaaagccaggtgtttctgaaaatgaacagcctgcagaccgatgataccgcgatttattattgcgcgaaacattattattatggcggcagctatgcgatggattattggggccagggcaccagcgtgaccgtgagcagcgcggcggcgccggcgccgcgcccgccgaccccggcgccgaccattgcgagccagccgctgagcctgcgcccggaagcgtgccgcccggcggcgggcggcgcggtgcatacccgcggcctggattttgtgcagccgatggcgctgattgtgctgggcggcgtggcgggcctgctgctgtttattggcctgggcatttttttttgcgtgcgctgccgcccgcgccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 22 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAPAPRPPTPAPTIASQPLSLRPEACRPAAGGAVHTRGLDFVQPMALIVLGGVAGLLLFIGLGIFFCVRCRPRRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1548 bp, synthetic construct
  source 1..1548: mol_type = other DNA
gaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgca

SEQ ID NO: 24 - protein, 516 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAA

SEQ ID NO: 25 - DNA, 2283 bp, synthetic construct
  source 1..2283: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 26 - protein, 761 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 726 bp, synthetic construct
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 28 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 29 - DNA, 1485 bp, synthetic construct
  source 1..1485: mol_type = other DNA
atgcttctcctggtcacctccctgctcctctgcgaactgcctcaccctgccttccttctgattcctgacactgacattcagatgactcagaccacctcttccttgtccgcgtcactgggagacagagtgaccatctcgtgtcgcgcaagccaggatatctccaagtacctgaactggtaccaacagaagcccgacgggactgtgaagctgctgatctaccacacctcacgcctgcacagcggagtgccaagcagattctccggctccggctcgggaaccgattactcgcttaccattagcaacctcgagcaggaggacatcgctacctacttctgccagcaaggaaataccctgccctacaccttcggcggaggaaccaaattggaaatcaccggctccacgagcggctccgggaagcctggttccggggaaggctccactaagggtgaagtgaagctccaggagtccggccccggcctggtggcgccgtcgcaatcactctctgtgacctgtaccgtgtcgggagtgtccctgcctgattacggcgtgagctggattcggcagccgccgcggaagggcctggaatggctgggtgtcatctggggatccgagactacctactacaactcggccctgaagtcccgcctgactatcatcaaagacaactcgaagtcccaggtctttctgaagatgaactccctgcaaactgacgacaccgccatctattactgtgctaagcactactactacggtggaagctatgctatggactactggggccaggggacatccgtgacagtcagctccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 30 - protein, 495 aa, synthetic construct
MLLLVTSLLLCELPHPAFLLIPDTDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 1470 bp, synthetic construct
  source 1..1470: mol_type = other DNA
atgcttctcctggtcacctccctgctcctctgcgaactgcctcaccctgccttccttctgattcctgacattcagatgactcagaccacctcttccttgtccgcgtcactgggagacagagtgaccatctcgtgtcgcgcaagccaggatatctccaagtacctgaactggtaccaacagaagcccgacgggactgtgaagctgctgatctaccacacctcacgcctgcacagcggagtgccaagcagattctccggctccggctcgggaaccgattactcgcttaccattagcaacctcgagcaggaggacatcgctacctacttctgccagcaaggaaataccctgccctacaccttcggcggaggaaccaaattggaaatcaccggcggaggaggctccgggggaggaggttccgggggcgggggttccgaagtgaagctccaggagtccggccccggcctggtggcgccgtcgcaatcactctctgtgacctgtaccgtgtcgggagtgtccctgcctgattacggcgtgagctggattcggcagccgccgcggaagggcctggaatggctgggtgtcatctggggatccgagactacctactacaactcggccctgaagtcccgcctgactatcatcaaagacaactcgaagtcccaggtctttctgaagatgaactccctgcaaactgacgacaccgccatctattactgtgctaagcactactactacggtggaagctatgctatggactactgggggcaaggcacttcggtgactgtgtcaagcgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 32 - protein, 490 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1548 bp, synthetic construct
  source 1..1548: mol_type = other DNA
gacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccggaggcggcggcagcggcgggggagggtccggagggggtggttctggtggaggaggatcgggaggcggtggcagcgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgca

SEQ ID NO: 34 - protein, 516 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKAAA

SEQ ID NO: 35 - DNA, 2283 bp, synthetic construct
  source 1..2283: mol_type = other DNA
atgctccttctcgtgacctccctgcttctctgcgaactgccccatcctgccttcctgctgattcccgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccggaggcggcggcagcggcgggggagggtccggagggggtggttctggtggaggaggatcgggaggcggtggcagcgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 36 - protein, 761 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 732 bp, synthetic construct
  source 1..732: mol_type = other DNA
gaggtccagctggtacagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatttatcgtcagtggctggaccctttaactactggggccagggcaccctggtcaccgtctcctcaggaggtggcgggtctggtggaggcggtagcggcggtggcggatcctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaggatgaggctgactattactgtaactcccgggacagcagtggtaaccatctggtattcggcggaggcacccagctgaccgtcctcggt

SEQ ID NO: 38 - protein, 244 aa, synthetic construct
EVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLG

SEQ ID NO: 39 - DNA, 1476 bp, synthetic construct
  source 1..1476: mol_type = other DNA
atgctgctgctggtgaccagcctgctgctgtgcgaactgccgcatccggcgtttctgctgattccggaggtccagctggtacagtctgggggaggcttggtacagcctggggggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatttatcgtcagtggctggaccctttaactactggggccagggcaccctggtcaccgtctcctcaggaggtggcgggtctggtggaggcggtagcggcggtggcggatcctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaggatgaggctgactattactgtaactcccgggacagcagtggtaaccatctggtattcggcggaggcacccagctgaccgtcctcggtgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 40 - protein, 492 aa, synthetic construct
MLLLVTSLLLCELPHPAFLLIPEVQLVQSGGGLVQPGGSLRLSCAASGFTFDDYAMHWVRQAPGKGLEWVSGISWNSGSIGYADSVKGRFTISRDNAKNSLYLQMNSLRAEDTALYYCAKDLSSVAGPFNYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTQLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 339 bp, synthetic construct
  source 1..339: mol_type = other DNA
gaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctca

SEQ ID NO: 42 - protein, 113 aa, synthetic construct
EVQLVESGGGLVQPGGSLRLSCAASGFTFSSYGMSWVRQAPRQGLEWVANIKQDGSEKYYADSVKGRFTISRDNSKNTLYLQMNSLRAEDTATYYCAKENVDWGQGTLVTVSS

SEQ ID NO: 43 - DNA, 1083 bp, synthetic construct
  source 1..1083: mol_type = other DNA
atgctgctgctggtgaccagcctgctgctgtgcgaactgccgcatccggcgtttctgctgattccggaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctcagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 44 - protein, 361 aa, synthetic construct
MLLLVTSLLLCELPHPAFLLIPEVQLVESGGGLVQPGGSLRLSCAASGFTFSSYGMSWVRQAPRQGLEWVANIKQDGSEKYYADSVKGRFTISRDNSKNTLYLQMNSLRAEDTATYYCAKENVDWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 797 bp, synthetic construct
  source 1..797: mol_type = other DNA
atggcactgcccgtgaccgccctgcttctgccgcttgcacttctgctgcacgccgctaggccccaagtcaccctcaaagagtcagggccaggaatcctcaagccctcacagactctgtctcttacttgctcattcagcggattcagcctttccacctctggtatgggcgtggggtggattaggcaacctagcggaaaggggcttgaatggctggcccacatctggtgggacgacgacaagtactacaacccctcactgaagtcccagctcactatttccaaagatacttcccggaatcaggtgttcctcaagattacctctgtcgacaccgctgataccgccacttactattgttcacgcagaccgagaggtaccatggacgcaatggactactggggacagggcaccagcgtgaccgtgtcatctggcggtggagggtcaggaggtggaggtagcggaggcggtgggtccgacattgtcatgacccaggccgccagcagcctgagcgcttcactgggcgacagggtgaccatcagctgtcgcgcatcacaagatatctctaagtatcttaattggtaccagcaaaagccggatggaaccgtgaagctgctgatctactacacctcacggctgcattctggagtgcctagccgctttagcggatctgggtccggtactgactacagcctcaccattagaaaccttgaacaggaggacatcgcaacttatttctgccaacaggtctatactctgccgtggaccttcggcggaggtaccaaactggagattaagtccgg

SEQ ID NO: 46 - protein, 265 aa, synthetic construct
MALPVTALLLPLALLLHAARPQVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCSRRPRGTMDAMDYWGQGTSVTVSSGGGGSGGGGSGGGGSDIVMTQAASSLSASLGDRVTISCRASQDISKYLNWYQQKPDGTVKLLIYYTSRLHSGVPSRFSGSGSGTDYSLTIRNLEQEDIATYFCQQVYTLPWTFGGGTKLEIKS

SEQ ID NO: 47 - DNA, 1170 bp, synthetic construct
  source 1..1170: mol_type = other DNA
atggcactgcccgtgaccgccctgcttctgccgcttgcacttctgctgcacgccgctaggccccaagtcaccctcaaagagtcagggccaggaatcctcaagccctcacagactctgtctcttacttgctcattcagcggattcagcctttccacctctggtatgggcgtggggtggattaggcaacctagcggaaaggggcttgaatggctggcccacatctggtgggacgacgacaagtactacaacccctcactgaagtcccagctcactatttccaaagatacttcccggaatcaggtgttcctcaagattacctctgtcgacaccgctgataccgccacttactattgttcacgcagaccgagaggtaccatggacgcaatggactactggggacagggcaccagcgtgaccgtgtcatctggcggtggagggtcaggaggtggaggtagcggaggcggtgggtccgacattgtcatgacccaggccgccagcagcctgagcgcttcactgggcgacagggtgaccatcagctgtcgcgcatcacaagatatctctaagtatcttaattggtaccagcaaaagccggatggaaccgtgaagctgctgatctactacacctcacggctgcattctggagtgcctagccgctttagcggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 48 - protein, 490 aa, synthetic construct
MALPVTALLLPLALLLHAARPQVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCSRRPRGTMDAMDYWGQGTSVTVSSGGGGSGGGGSGGGGSDIVMTQAASSLSASLGDRVTISCRASQDISKYLNWYQQKPDGTVKLLIYYTSRLHSGVPSRFSGSGSGTDYSLTIRNLEQEDIATYFCQQVYTLPWTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 1467 bp, synthetic construct
  source 1..1467: mol_type = other DNA
atgctgctgctggtcaccagcctgctgctgtgcgagctccctcaccccgcctttctgcttatcccggacattcagatgacacagaccacctcgagcttgtccgcgtcgctgggcgatcgcgtgaccatctcctgccgggcctcccaagacatttcaaagtatctcaactggtaccagcagaagccggacggaaccgtgaaactgctgatctaccataccagccgcctgcactccggcgtgccgtcccgcttctccggatcgggttccggaactgactactcactgactatctccaacttggaacaagaggacatcgccacttacttctgtcaacaaggaaatacccttccctacaccttcggggggggtaccaagctggagatcactgggggcggaggctccggtggaggcggatccggcggtggagggagcgaagtcaagctgcaggaatcaggaccaggactcgtggcgccatcccagtccctgtcggtgacctgtactgtctccggagtcagcctccccgattacggagtgtcatggattaggcaacccccaagaaaagggctggaatggctcggagtgatctggggctccgaaaccacctactacaactcggcgctgaagtcccggctgaccatcatcaaggacaactccaagagccaagtgttcttgaagatgaacagcttgcagaccgacgataccgcaatctactactgtgccaagcactattactacggggggtcttacgccatggactactggggacagggcacctccgtgactgtgtcgtccgcggccgcgcccgcccctcggcccccgactcctgccccgacgatcgcttcccaacctctctcgctgcgcccggaagcatgccggcccgccgccggtggcgctgtccacactcgcggactggactttgataccgcactggcggccgtgatctgtagcgccctggccaccgtgctgctggcgctgctcatcctttgcgtgatctactgcaagcggcagcctaggcgaaagaagctcctctacattttcaagcaacccttcatgcgccccgtgcaaaccacccaggaggaggatggatgctcatgccggttccctgaggaagaagagggcggttgcgagctcagagtgaaattcagccggtcggctgacgccccggcgtaccagcagggccagaaccagctgtacaatgagctcaacctggggcgccgcgaagagtacgacgtgctggacaagaggagaggcagagatccggaaatgggcggaaagccaaggcggaagaacccgcaggaaggtctttacaacgaactgcagaaggacaagatggccgaggcctactccgagattgggatgaagggagaaagacggaggggaaagggacatgacggactttaccagggcctgagcactgccacgaaggacacctatgatgccctgcacatgcaggcgctgccgcctcgg

SEQ ID NO: 50 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAPAPRPPTPAPTIASQPLSLRPEACRPAAGGAVHTRGLDFDTALAAVICSALATVLLALLILCVIYCKRQPRRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 2280 bp, synthetic construct
  source 1..2280: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccggtcgaagaggtccagactcttgcactccgactacatgaacatgactcctagaaggcccggacccactagaaagcactaccagccgtacgcccctcctcgggatttcgccgcataccggtccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 52 - protein, 760 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 3858 bp, synthetic construct
  source 1..3858: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccggtcgaagaggtccagactcttgcactccgactacatgaacatgactcctagaaggcccggacccactagaaagcactaccagccgtacgcccctcctcgggatttcgccgcataccggtccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatgcttctattagtgacttcccttttgctgtgcgagttgccacaccccgccttcctgcttattccccaggtacagctccagcagagtggcccagggctcgtgaagccaagccagacgctgtccctgacttgtgcaatttcaggggattcagtttcatcaaatagcgcggcgtggaattggattcgacaatctccttcccgagggttggaatggcttggacgaacatattacagatccaaatggtataacgactatgcggtatcagtaaagtcaagaataaccattaaccccgacacaagcaagaaccaattctctttgcagcttaactctgtcacgccagaagacacggcagtctattattgcgctcgcgaggtaacgggtgacctggaagacgcttttgacatttgggggcagggtacgatggtgacagtcagttcagggggcggtgggagtgggggagggggtagcggggggggagggtcagacattcagatgacccagtccccttcatccttgtctgcctccgtcggtgacagggtgacaataacatgcagagcaagccaaacaatctggagctatctcaactggtaccagcagcgaccaggaaaagcgccaaacctgctgatttacgctgcttcctccctccaatcaggcgtgcctagtagatttagcggtaggggctccggcaccgattttacgctcactataagctctcttcaagcagaagattttgcgacttattactgccagcagtcctatagtatacctcagactttcggacagggtaccaagttggagattaaggctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgcaagaggggccggaagaagctgctttacatcttcaagcagccgttcatgcggcccgtgcagacgactcaggaagaggacggatgctcgtgcagattccctgaggaggaagaggggggatgcgaactgagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacgg

SEQ ID NO: 54 - protein, 1286 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 3852 bp, synthetic construct
  source 1..3852: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccggtcgaagaggtccagactcttgcactccgactacatgaacatgactcctagaaggcccggacccactagaaagcactaccagccgtacgcccctcctcgggatttcgccgcataccggtccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatgttgctgctcgtgacctcgctccttctgtgcgagctgccccatccggcttttctgctcatccctcaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgcaagaggggccggaagaagctgctttacatcttcaagcagccgttcatgcggcccgtgcagacgactcaggaagaggacggatgctcgtgcagattccctgaggaggaagaggggggatgcgaactgagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacgg

SEQ ID NO: 56 - protein, 1284 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMLLLVTSLLLCELPHPAFLLIPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKASATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57-58 - skipped (2 entries)

SEQ ID NO: 59 - DNA, 3855 bp, synthetic construct
  source 1..3855: mol_type = other DNA
atgcttcttttggtgacttcccttttgctgtgcgagttgccacaccccgccttcctgcttattccccaggtacagctccagcagagtggcccagggctcgtgaagccaagccagacgctgtccctgacttgtgcaatttcaggggattcagtttcatcaaatagcgcggcgtggaattggattcgacaatctccttcccgagggttggaatggcttggacgaacatattacagatccaaatggtataacgactatgcggtatcagtaaagtcaagaataaccattaaccccgacacaagcaagaaccaattctctttgcagcttaactctgtcacgccagaagacacggcagtctattattgcgctcgcgaggtaacgggtgacctggaagacgcttttgacatttgggggcagggtacgatggtgacagtcagttcagggggcggtgggagtgggggagggggtagcggggggggagggtcagacattcagatgacccagtccccttcatccttgtctgcctccgtcggtgacagggtgacaataacatgcagagcaagccaaacaatctggagctatctcaactggtaccagcagcgaccaggaaaagcgccaaacctgctgatttacgctgcttcctccctccaatcaggcgtgcctagtagatttagcggtaggggctccggcaccgattttacgctcactataagctctcttcaagcagaagattttgcgacttattactgccagcagtcctatagtatacctcagactttcggacagggtaccaagttggagattaaggcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatggccctgcccgtcactgcgctgcttcttccacttgcgcttctgctgcacgcagcgcgcccggaagtccagctccagcaaagcggagccgaactcgtgaagccgggggcctccgtgaagatgagctgcaaggcatccggctacaccttcactagctacaacatgcactgggtgaagcagactccgggtcaagggctggagtggattggggcgatctacccgggcaacggcgacacctcctacaaccaaaagttcaaggggaaggctactcttacggcggacaagtcgtccagcaccgcatacatgcaactctcctccctgacctccgaggactcggcggactactactgcgcccggagcaactactacggttcctcctactggttcttcgacgtgtggggtgccggaactactgtgactgtgtcctccggtggtggcggatcaggcggcgggggatccggcggtggaggatccgacattgtgctgactcagtcccccgcaatcctttcggcctcccccggagagaaggtcacgatgacttgcagggcttcgtcctccgtgaactacatggattggtaccaaaagaagcccgggtcgtcgcctaagccgtggatctacgctacctcaaacctggcttccggcgtccctgcgcggttcagcggctcggggagcggtacctcatactcactcaccatctcccgggtggaggccgaagatgcggccacctattattgccaacagtggtccttcaatccgcccaccttcggggggggaaccaagctcgagatcaaggggggtggcggctcagggggaggcggaagcggagggggtggctcgggcggcggcggttccggcggcggagggtccgatatccaaatgacccagactactagctcgttgagcgcctcgctcggcgacagagtgaccattagctgcagggcatcccaggacatttcaaagtacctgaactggtaccaacagaagcccgacggaactgtgaagctcctgatctaccacacctcccggctgcactccggagtcccgtcgagattttccggctccggaagcggaaccgattattcgctcaccatttctaacctggaacaggaggacattgccacttacttctgtcaacaaggaaacactctgccttacacctttggtggcggaaccaagttggaaattaccggctccacctccggatccggaaagcctggatccggagagggatcaaccaagggagaagtgaagctgcaggagagcgggcccggccttgtcgccccgagccagtccttgtccgtgacctgtactgtctccggagtcagcctgccggactacggggtgtcctggatccgccagccgcctcgcaagggcctggagtggctcggcgtgatctggggatccgaaacgacttactacaactcggccctcaagtcgaggctcactattatcaaggacaactcgaagtcccaggtgttcctcaagatgaactcgctgcaaaccgacgacacagcgatctactactgtgcaaagcattactactacggaggcagctacgcaatggactactggggacagggaacctccgtgactgtctctagcgctagcgcgaccactacgcccgccccccgcccacctacccccgccccgaccattgcgagccaaccgttgtcactccgcccggaagcctgccgccccgccgctggcggagccgtgcacacccggggactggacttcgcatgcgacatctacatttgggccccgctggctggaacctgtggagtcctgctgctctccctcgtgatcactctgtactgccggtcgaagcgctcaagactgctgcactcagactacatgaacatgactcctcggcggccggggccgactcggaagcactaccagccttacgcacccccgagagatttcgcggcctaccgctcccgggtcaagttttcccggtctgccgacgctccggcgtaccagcaggggcagaaccagctctacaatgagctgaatctgggtcggagagaagagtacgatgtgctggataagcggagaggcagagatccagaaatgggaggaaagcctcggagaaagaacccacaggagggactgtataatgagctgcagaaggacaaaatggccgaagcctacagcgagatcggcatgaagggagagcggcgcagagggaagggacatgacggcctgtaccagggtctgagcaccgcgactaaggacacctacgatgcccttcatatgcaagcactccctccgcgc

SEQ ID NO: 60 - protein, 1285 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRVDMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 3849 bp, synthetic construct
  source 1..3849: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgcaagcgaggtaggaagaaattgctttacatttttaagcagccgttcatgcgaccagtacagactactcaagaagaagatgggtgctcttgtcggttcccggaagaagaagagggtggttgcgagttgagggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctagacgggcaaagcggggctcaggggcgactaacttttcactgttgaagcaggccggggatgtggaggagaatcctggtcctagagctaagcgagtagacatggccctgcccgtcactgcgctgcttcttccacttgcgcttctgctgcacgcagcgcgcccggaagtccagctccagcaaagcggagccgaactcgtgaagccgggggcctccgtgaagatgagctgcaaggcatccggctacaccttcactagctacaacatgcactgggtgaagcagactccgggtcaagggctggagtggattggggcgatctacccgggcaacggcgacacctcctacaaccaaaagttcaaggggaaggctactcttacggcggacaagtcgtccagcaccgcatacatgcaactctcctccctgacctccgaggactcggcggactactactgcgcccggagcaactactacggttcctcctactggttcttcgacgtgtggggtgccggaactactgtgactgtgtcctccggtggtggcggatcaggcggcgggggatccggcggtggaggatccgacattgtgctgactcagtcccccgcaatcctttcggcctcccccggagagaaggtcacgatgacttgcagggcttcgtcctccgtgaactacatggattggtaccaaaagaagcccgggtcgtcgcctaagccgtggatctacgctacctcaaacctggcttccggcgtccctgcgcggttcagcggctcggggagcggtacctcatactcactcaccatctcccgggtggaggccgaagatgcggccacctattattgccaacagtggtccttcaatccgcccaccttcggggggggaaccaagctcgagatcaaggggggtggcggctcagggggaggcggaagcggagggggtggctcgggcggcggcggttccggcggcggagggtccgatatccaaatgacccagactactagctcgttgagcgcctcgctcggcgacagagtgaccattagctgcagggcatcccaggacatttcaaagtacctgaactggtaccaacagaagcccgacggaactgtgaagctcctgatctaccacacctcccggctgcactccggagtcccgtcgagattttccggctccggaagcggaaccgattattcgctcaccatttctaacctggaacaggaggacattgccacttacttctgtcaacaaggaaacactctgccttacacctttggtggcggaaccaagttggaaattaccggctccacctccggatccggaaagcctggatccggagagggatcaaccaagggagaagtgaagctgcaggagagcgggcccggccttgtcgccccgagccagtccttgtccgtgacctgtactgtctccggagtcagcctgccggactacggggtgtcctggatccgccagccgcctcgcaagggcctggagtggctcggcgtgatctggggatccgaaacgacttactacaactcggccctcaagtcgaggctcactattatcaaggacaactcgaagtcccaggtgttcctcaagatgaactcgctgcaaaccgacgacacagcgatctactactgtgcaaagcattactactacggaggcagctacgcaatggactactggggacagggaacctccgtgactgtctctagcgctagcgcgaccactacgcccgccccccgcccacctacccccgccccgaccattgcgagccaaccgttgtcactccgcccggaagcctgccgccccgccgctggcggagccgtgcacacccggggactggacttcgcatgcgacatctacatttgggccccgctggctggaacctgtggagtcctgctgctctccctcgtgatcactctgtactgccggtcgaagcgctcaagactgctgcactcagactacatgaacatgactcctcggcggccggggccgactcggaagcactaccagccttacgcacccccgagagatttcgcggcctaccgctcccgggtcaagttttcccggtctgccgacgctccggcgtaccagcaggggcagaaccagctctacaatgagctgaatctgggtcggagagaagagtacgatgtgctggataagcggagaggcagagatccagaaatgggaggaaagcctcggagaaagaacccacaggagggactgtataatgagctgcagaaggacaaaatggccgaagcctacagcgagatcggcatgaagggagagcggcgcagagggaagggacatgacggcctgtaccagggtctgagcaccgcgactaaggacacctacgatgcccttcatatgcaagcactccctccgcgc

SEQ ID NO: 62 - protein, 1283 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRVDMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63-64 - skipped (2 entries)

SEQ ID NO: 65 - DNA, 1476 bp, synthetic construct
  source 1..1476: mol_type = other DNA
atgcttcttttggtgacttcccttttgctgtgcgagttgccacaccccgccttcctgcttattccccaggtacagcttcaacagagtgggccgggactggtgaaacactcccaaacactttctctgacgtgcgctatatcaggtgactctgtttcatctaattctgctgcgtggaactggattcgacaatctcccagtcgcgggttggaatggctgggacgaacatattatcggtctaagtggtataacgattatgctgtatctgttaaatctcgaattacgattaatcctgacacctccaagaaccagttctccctccagttgaactcagtcacaccggaagacactgcggtctactattgcgctcaagaagtcgagccacatgatgcattcgacatctggggccagggaacgatggtcaccgtcagcagtggcggcggcggatctgggggtggcggttctggcggtggaggatcagacatacaaatgacgcagagtccctcaagtgtgtacgcgagtgtgggggataaggtaactattacgtgcagagcgtcacaggatgttagtggatggcttgcctggtatcagcagaagccaggccttgctccacagctccttatcagtggtgcttctacacttcagggcgaggttccgagtagattctctggttctggatctggtactgacttcactcttacaatttcttctttgcaaccagaagactttgcgacttattactgccaacaggccaaatacttcccttatacatttggccaaggtaccaagttggagataaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 66 - protein, 492 aa, synthetic construct
MLLLVTSLLLCELPHPAFLLIPQVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSSGGGGSGGGGSGGGGSDIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1476 bp, synthetic construct
  source 1..1476: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 68 - protein, 492 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 1476 bp, synthetic construct
  source 1..1476: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgccattagcgggaactcagtctcgtccaattcggcggcctggaactggatccggcagtcaccatcaaggggcctggaatggctcgggcgcacttactaccggtccaaatggtataacgactacgccgtgtccgtgaagtcccggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatctttggcgccagcactcttcagggggaggtgccatcacgcttctccggaggtggttccggcaccgacttcaccctgaccatcagcagcctccagcctgaggacttcgccacttactactgccaacaggccaagtacttcccctataccttcggacaaggcactaagctggaaatcaaggcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 70 - protein, 492 aa, synthetic construct
MLLLVTSLLLCELPHPAFLLIPQVQLQQSGPGLVKPSQTLSLTCAISGNSVSSNSAAWNWIRQSPSRGLEWLGRTYYRSKWYNDYAVSVKSRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGGGSGTDFTLTISSLQPEDFATYYCQQAKYFPYTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71-72 - skipped (2 entries)

SEQ ID NO: 73 - DNA, 2280 bp, synthetic construct; identical to SEQ ID NO: 51
  source 1..2280: mol_type = other DNA

SEQ ID NO: 74 - protein, 760 aa, synthetic construct; identical to SEQ ID NO: 52

SEQ ID NO: 75 - DNA, 1482 bp, synthetic construct; identical to SEQ ID NO: 9
  source 1..1482: mol_type = other DNA

SEQ ID NO: 76 - protein, 494 aa, synthetic construct; identical to SEQ ID NO: 10

SEQ ID NO: 77 - DNA, 66 bp, synthetic construct
  source 1..66: mol_type = other DNA
atgcttcttttggtgacttcccttttgctgtgcgagttgccacaccccgccttcctgcttattccc

SEQ ID NO: 78 - protein, 22 aa, synthetic construct
MLLLVTSLLLCELPHPAFLLIP

SEQ ID NO: 79 - DNA, 63 bp, synthetic construct
  source 1..63: mol_type = other DNA
atggccctgcccgtcactgcgctgcttcttccacttgcgcttctgctgcacgcagcgcgcccg

SEQ ID NO: 80 - protein, 21 aa, synthetic construct
MALPVTALLLPLALLLHAARP

SEQ ID NO: 81 - DNA, 216 bp, synthetic construct
  source 1..216: mol_type = other DNA
gcggccgctaccacaacccctgcgccccggcctcctacccccgcacccacgattgcttctcaacctctttcactccgacctgaggcttgtagacctgcagccgggggtgccgtccacacacggggactcgacttcgcttgtgatatatatatttgggcgcccctggccggcacttgtggagttcttttgctctctcttgttatcacattgtactgc

SEQ ID NO: 82 - protein, 72 aa, synthetic construct
AAATTTPAPRPPTPAPTIASQPLSLRPEACRPAAGGAVHTRGLDFACDIYIWAPLAGTCGVLLLSLVITLYC

SEQ ID NO: 83 - DNA, 126 bp, synthetic construct
  source 1..126: mol_type = other DNA
aagcgaggtaggaagaaattgctttacatttttaagcagccgttcatgcgaccagtacagactactcaagaagaagatgggtgctcttgtcggttcccggaagaagaagagggtggttgcgagttg

SEQ ID NO: 84 - protein, 42 aa, synthetic construct; identical to SEQ ID NO: 18

SEQ ID NO: 85 - DNA, 123 bp, synthetic construct
  source 1..123: mol_type = other DNA
cggtcgaagcgctcaagactgctgcactcagactacatgaacatgactcctcggcggccggggccgactcggaagcactaccagccttacgcacccccgagagatttcgcggcctaccgctcc

SEQ ID NO: 86 - protein, 41 aa, synthetic construct
RSKRSRLLHSDYMNMTPRRPGPTRKHYQPYAPPRDFAAYRS

SEQ ID NO: 87 - DNA, 336 bp, synthetic construct
  source 1..336: mol_type = other DNA
agggtgaagttctcccgctctgccgacgcaccggcatatcagcagggacaaaaccagctctacaacgaattgaacctgggtcggcgggaagaatatgacgtgctcgataagcggcggggtcgcgacccagaaatgggaggcaaaccgcgcaggaaaaatccacaggagggactttataacgaacttcaaaaggataagatggcagaggcatacagcgaaatcgggatgaaaggcgagagaagaagggggaaagggcacgatggtctttaccaggggctttctaccgcgacgaaggatacctacgatgctctccatatgcaagcacttcctcctaga

SEQ ID NO: 88 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 91 bp, synthetic construct
  source 1..91: mol_type = other DNA
cgggcaaagcggggctcaggggcgactaacttttcactgttgaagcaggccggggatgtggaggagaatcctggtcctagagctaagcgag

SEQ ID NO: 90 - protein, 30 aa, synthetic construct
RAKRGSGATNFSLLKQAGDVEENPGPRAKR

SEQ ID NO: 91-94 - skipped (4 entries)

SEQ ID NO: 95 - DNA, 366 bp, synthetic construct
  source 1..366: mol_type = other DNA
caggtacagcttcaacagagtgggccgggactggtgaaacactcccaaacactttctctgacgtgcgctatatcaggtgactctgtttcatctaattctgctgcgtggaactggattcgacaatctcccagtcgcgggttggaatggctgggacgaacatattatcggtctaagtggtataacgattatgctgtatctgttaaatctcgaattacgattaatcctgacacctccaagaaccagttctccctccagttgaactcagtcacaccggaagacactgcggtctactattgcgctcaagaagtcgagccacatgatgcattcgacatctggggccagggaacgatggtcaccgtcagcagt

SEQ ID NO: 96 - protein, 122 aa, synthetic construct
QVQLQQSGPGLVKHSQTLSLTCAISGDSVSSNSAAWNWIRQSPSRGLEWLGRTYYRSKWYNDYAVSVKSRITINPDTSKNQFSLQLNSVTPEDTAVYYCAQEVEPHDAFDIWGQGTMVTVSS

SEQ ID NO: 97 - DNA, 321 bp, synthetic construct
  source 1..321: mol_type = other DNA
gacatacaaatgacgcagagtccctcaagtgtgtacgcgagtgtgggggataaggtaactattacgtgcagagcgtcacaggatgttagtggatggcttgcctggtatcagcagaagccaggccttgctccacagctccttatcagtggtgcttctacacttcagggcgaggttccgagtagattctctggttctggatctggtactgacttcactcttacaatttcttctttgcaaccagaagactttgcgacttattactgccaacaggccaaatacttcccttatacatttggccaaggtaccaagttggagataaag

SEQ ID NO: 98 - protein, 107 aa, synthetic construct
DIQMTQSPSSVYASVGDKVTITCRASQDVSGWLAWYQQKPGLAPQLLISGASTLQGEVPSRFSGSGSGTDFTLTISSLQPEDFATYYCQQAKYFPYTFGQGTKLEIK

SEQ ID NO: 99 - DNA, 366 bp, synthetic construct
  source 1..366: mol_type = other DNA
caagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtcc

SEQ ID NO: 100 - protein, 122 aa, synthetic construct
QVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSS

SEQ ID NO: 101 - DNA, 321 bp, synthetic construct
  source 1..321: mol_type = other DNA
gatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaag

SEQ ID NO: 102 - protein, 107 aa, synthetic construct
DIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIK

SEQ ID NO: 103 - DNA, 366 bp, synthetic construct
  source 1..366: mol_type = other DNA
caagtgcagctgcagcagtccggtcctggactggtcaagccgtcccagactctgagcctgacttgcgccattagcgggaactcagtctcgtccaattcggcggcctggaactggatccggcagtcaccatcaaggggcctggaatggctcgggcgcacttactaccggtccaaatggtataacgactacgccgtgtccgtgaagtcccggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtcc

SEQ ID NO: 104 - protein, 122 aa, synthetic construct
QVQLQQSGPGLVKPSQTLSLTCAISGNSVSSNSAAWNWIRQSPSRGLEWLGRTYYRSKWYNDYAVSVKSRITINPDTSKNQFSLQLNSVTPEDTAVYYCAQEVEPQDAFDIWGQGTMVTVSS

SEQ ID NO: 105 - DNA, 321 bp, synthetic construct
  source 1..321: mol_type = other DNA
gatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatctttggcgccagcactcttcagggggaggtgccatcacgcttctccggaggtggttccggcaccgacttcaccctgaccatcagcagcctccagcctgaggacttcgccacttactactgccaacaggccaagtacttcccctataccttcggacaaggcactaagctggaaatcaag

SEQ ID NO: 106 - protein, 107 aa, synthetic construct
DIQMTQSPSSVSASVGDKVTITCRASQDVSGWLAWYQQKPGLAPQLLIFGASTLQGEVPSRFSGGGSGTDFTLTISSLQPEDFATYYCQQAKYFPYTFGQGTKLEIK

SEQ ID NO: 107 - protein, 18 aa, synthetic construct
GSTSGSGKPGSGEGSTKG

SEQ ID NO: 108 - protein, 25 aa, synthetic construct
GGGGSGGGGSGGGGSGGGGSGGGGS

SEQ ID NO: 109 - DNA, 3711 bp, synthetic construct
  source 1..3711: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaacgaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgctggctgacaaaaaagaagtattcatctagtgtacatgatccgaacggtgaatacatgttcatgcgcgcggtgaacacggccaagaagagcagactgaccgacgtaacccttagagtgaagtttagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggccccaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgcagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttaagcacagcaacaaaggatacttacgacgctcttcacatgcaagctctgccaccacgg

SEQ ID NO: 110 - protein, 1237 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKASATTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 3717 aa, synthetic construct
ATGCTCCTTCTCGTGACCTCCCTGCTTCTCTGCGAACTGCCCCATCCTGCCTTCCTGCTGATTCCCGAGGTGCAGTTGCAACAGTCAGGAGCTGAACTGGTCAAGCCAGGAGCCAGCGTGAAGATGAGCTGCAAGGCCTCCGGTTACACCTTCACCTCCTACAACATGCACTGGGTGAAACAGACCCCGGGACAAGGGCTCGAATGGATTGGCGCCATCTACCCCGGGAATGGCGATACTTCGTACAACCAGAAGTTCAAGGGAAAGGCCACCCTGACCGCCGACAAGAGCTCCTCCACCGCGTATATGCAGTTGAGCTCCCTGACCTCCGAGGACTCCGCCGACTACTACTGCGCACGGTCCAACTACTATGGAAGCTCGTACTGGTTCTTCGATGTCTGGGGGGCCGGCACCACTGTGACCGTCAGCTCCGGGGGCGGAGGATCCGGTGGAGGCGGAAGCGGGGGTGGAGGATCCGACATTGTGCTGACTCAGTCCCCGGCAATCCTGTCGGCCTCACCGGGCGAAAAGGTCACGATGACTTGTAGAGCGTCGTCCAGCGTGAACTACATGGATTGGTACCAAAAGAAGCCTGGATCGTCACCCAAGCCTTGGATCTACGCTACATCTAACCTGGCCTCCGGCGTGCCAGCGCGGTTCAGCGGGTCCGGCTCGGGCACCTCATACTCGCTGACCATCTCCCGCGTGGAGGCTGAGGACGCCGCGACCTACTACTGCCAGCAGTGGTCCTTCAACCCGCCGACTTTTGGAGGCGGTACTAAGCTGGAGATCAAAGGAGGCGGCGGCAGCGGCGGGGGAGGGTCCGGAGGGGGTGGTTCTGGTGGAGGAGGATCGGGAGGCGGTGGCAGCGACATTCAGATGACTCAGACCACCTCCTCCCTGTCCGCCTCCCTGGGCGACCGCGTGACCATCTCATGCCGCGCCAGCCAGGACATCTCGAAGTACCTCAACTGGTACCAGCAGAAGCCCGACGGAACCGTGAAGCTCCTGATCTACCACACCTCCCGGCTGCACAGCGGAGTGCCGTCTAGATTCTCGGGTTCGGGGTCGGGAACTGACTACTCCCTTACTATTTCCAACCTGGAGCAGGAGGATATTGCCACCTACTTCTGCCAACAAGGAAACACCCTGCCGTACACTTTTGGCGGGGGAACCAAGCTGGAAATCACTGGCAGCACATCCGGTTCCGGGAAGCCCGGCTCCGGAGAGGGCAGCACCAAGGGGGAAGTCAAGCTGCAGGAATCAGGACCTGGCCTGGTGGCCCCGAGCCAGTCACTGTCCGTGACTTGTACTGTGTCCGGAGTGTCGCTCCCGGATTACGGAGTGTCCTGGATCAGGCAGCCACCTCGGAAAGGATTGGAATGGCTCGGAGTCATCTGGGGTTCCGAAACCACCTATTACAACTCGGCACTGAAATCCAGGCTCACCATTATCAAGGATAACTCCAAGTCACAAGTGTTCCTGAAGATGAATAGCCTGCAGACTGACGACACGGCGATCTACTATTGCGCCAAGCACTACTACTACGGCGGATCCTACGCTATGGACTACTGGGGCCAGGGGACCAGCGTGACCGTGTCATCCGCGGCCGCAACGACCACTCCAGCACCGAGACCGCCAACCCCCGCGCCTACCATCGCAAGTCAACCACTTTCTCTCAGGCCTGAAGCGTGCCGACCTGCAGCTGGTGGGGCAGTACATACCAGGGGTTTGGACTTCGCATGTGACGTGGCGGCAATTCTCGGCCTGGGACTTGTCCTTGGTCTGCTTGGTCCGCTCGCAATACTTCTGGCCTTGTACCTGCTCCGCAGAGACCAAAGACTTCCGCCCGACGCCCACAAGCCCCCAGGAGGAGGTTCCTTCAGAACGCCTATACAAGAAGAACAAGCAGATGCCCACTCTACCCTGGCTAAAATCAGGGTGAAGTTTAGCCGCTCAGCCGATGCACCGGCCTACCAGCAGGGACAGAACCAGCTCTACAACGAGCTCAACCTGGGTCGGCGGGAAGAATATGACGTGCTGGACAAACGGCGCGGCAGAGATCCGGAGATGGGGGGAAAGCCGAGGAGGAAGAACCCTCAAGAGGGCCTGTACAACGAACTGCAGAAGGACAAGATGGCGGAAGCCTACTCCGAGATCGGCATGAAGGGAGAACGCCGGAGAGGGAAGGGTCATGACGGACTGTACCAGGGCCTGTCAACTGCCACTAAGGACACTTACGATGCGCTCCATATGCAAGCTTTGCCCCCGCGGCGCGCGAAACGCGGCAGCGGCGCGACCAACTTTAGCCTGCTGAAACAGGCGGGCGATGTGGAAGAAAACCCGGGCCCGCGAGCAAAGAGGAATATTATGGCTCTGCCTGTTACGGCACTGCTCCTTCCGCTTGCATTGTTGTTGCACGCAGCGCGGCCCCAAGTGCAGCTGCAGCAGTCCGGTCCTGGACTGGTCAAGCCGTCCCAGACTCTGAGCCTGACTTGCGCAATTAGCGGGGACTCAGTCTCGTCCAATTCGGCGGCCTGGAACTGGATCCGGCAGTCACCATCAAGGGGCCTGGAATGGCTCGGGCGCACTTACTACCGGTCCAAATGGTATACCGACTACGCCGTGTCCGTGAAGAATCGGATCACCATTAACCCCGACACCTCGAAGAACCAGTTCTCACTCCAACTGAACAGCGTGACCCCCGAGGATACCGCGGTGTACTACTGCGCACAAGAAGTGGAACCGCAGGACGCCTTCGACATTTGGGGACAGGGAACGATGGTCACAGTGTCGTCCGGTGGAGGAGGTTCCGGAGGCGGTGGATCTGGAGGCGGAGGTTCGGATATCCAGATGACCCAGAGCCCCTCCTCGGTGTCCGCATCCGTGGGCGATAAGGTCACCATTACCTGTAGAGCGTCCCAGGACGTGTCCGGATGGCTGGCCTGGTACCAGCAGAAGCCAGGCTTGGCTCCTCAACTGCTGATCTTCGGCGCCAGCACTCTTCAGGGGGAAGTGCCATCACGCTTCTCCGGATCCGGTTCCGGCACCGACTTCACCCTGACCATCAGCAGCCTCCAGCCTGAGGACTTCGCCACTTACTACTGCCAACAGGCCAAGTACTTCCCCTATACCTTCGGAAGAGGCACTAAGCTGGAAATCAAGGCTAGCGCAACCACTACGCCTGCTCCGCGGCCTCCAACGCCCGCGCCCACGATAGCTAGTCAGCCGTTGTCTCTCCGACCAGAGGCGTGTAGACCGGCCGCTGGCGGAGCCGTACATACTCGCGGACTCGACTTCGCTTGCGACATCTACATTTGGGCACCCTTGGCTGGGACCTGTGGGGTGCTGTTGCTGTCCTTGGTTATTACGTTGTACTGCAGAGTCAAATTTTCCAGGTCCGCAGATGCCCCCGCGTACCAGCAAGGCCAGAACCAACTTTACAACGAACTGAACCTGGGTCGCCGGGAGGAATATGATGTGCTGGATAAACGAAGGGGGAGGGACCCTGAGATGGGAGGGAAACCTCGCAGGAAAAACCCGCAGGAAGGTTTGTACAACGAGTTGCAGAAGGATAAGATGGCTGAGGCTTACTCTGAAATAGGGATGAAGGGAGAGAGACGGAGAGGAAAAGGCCATGATGGCCTTTACCAGGGCTTAAGCACAGCAACAAAGGATACTTACGACGCTCTTCACATGCAAGCTCTGCCACCACGG

SEQ ID NO: 112 - protein, 1239 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKASATTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 3828 bp, synthetic construct
  source 1..3828: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaacgaccactccagcaccgagaccgccaacccccgcgcctaccatcgcaagtcaaccactttctctcaggcctgaagcgtgccgacctgcagctggtggggcagtacataccaggggtttggacttcgcatgtgacgtggcggcaattctcggcctgggacttgtccttggtctgcttggtccgctcgcaatacttctggccttgtacctgctccgcagagaccaaagacttccgcccgacgcccacaagcccccaggaggaggttccttcagaacgcctatacaagaagaacaagcagatgcccactctaccctggctaaaatcagggtgaagtttagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggccccaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgctggctgacaaaaaagaagtattcatctagtgtacatgatccgaacggtgaatacatgttcatgcgcgcggtgaacacggccaagaagagcagactgaccgacgtaacccttagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacgg

SEQ ID NO: 114 - protein, 1276 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKASATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 3855 bp, synthetic construct
  source 1..3855: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccaacggcgcaaataccgctccaataaaggcgaaagtccggtagaacccgcagaaccttgccactacagttgtcccagagaagaagagggttctacaatacctattcaagaggactataggaaaccagagcccgcatgtagtcccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggccccaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgctggctgacaaaaaagaagtattcatctagtgtacatgatccgaacggtgaatacatgttcatgcgcgcggtgaacacggccaagaagagcagactgaccgacgtaacccttagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacgg

SEQ ID NO: 116 - protein, 1285 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKASATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 2268 bp, synthetic construct
  source 1..2268: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgctggctgacaaaaaagaagtattcatctagtgtacatgatccgaacggtgaatacatgttcatgcgcgcggtgaacacggccaagaagagcagactgaccgacgtaacccttagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 118 - protein, 756 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1461 bp, synthetic construct
  source 1..1461: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgctggctgacaaaaaagaagtattcatctagtgtacatgatccgaacggtgaatacatgttcatgcgcgcggtgaacacggccaagaagagcagactgaccgacgtaacccttagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 120 - protein, 487 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKAAATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 2274 bp, synthetic construct
  source 1..2274: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaacgaccactccagcaccgagaccgccaacccccgcgcctaccatcgcaagtcaaccactttctctcaggcctgaagcgtgccgacctgcagctggtggggcagtacataccaggggtttggacttcgcatgtgacgtggcggcaattctcggcctgggacttgtccttggtctgcttggtccgctcgcaatacttctggccttgtacctgctccgcagagaccaaagacttccgcccgacgcccacaagcccccaggaggaggttccttcagaacgcctatacaagaagaacaagcagatgcccactctaccctggctaaaatcagggtgaagtttagccggtcagctgatgcacctgcatatcagcagggacagaaccagctgtacaatgagctgaacctcggacgaagagaggagtacgacgtgttggacaaaagacgaggtagagaccccgagatgggcggcaagccgagaagaaaaaacccacaagaagggctttataatgagcttcagaaagataagatggcagaggcctacagtgagattggcatgaagggcgaaagaaggaggggcaaaggacacgacggtctctaccaaggcctcagcacggctaccaaagatacgtatgacgcattgcatatgcaggcattgccgccccgc

SEQ ID NO: 122 - protein, 758 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 2301 bp, synthetic construct
  source 1..2301: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccaacggcgcaaataccgctccaataaaggcgaaagtccggtagaacccgcagaaccttgccactacagttgtcccagagaagaagagggttctacaatacctattcaagaggactataggaaaccagagcccgcatgtagtcccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 124 - protein, 767 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 1350 bp, synthetic construct
  source 1..1350: mol_type = other DNA
atgttgctgctcgtgacctcgctccttctgtgcgagctgccccatccggcttttctgctcatccctcaagtgcagctgcagcagtccggtcctggactggtcaagccgtcccagactctgagcctgacttgcgcaattagcggggactcagtctcgtccaattcggcggcctggaactggatccggcagtcaccatcaaggggcctggaatggctcgggcgcacttactaccggtccaaatggtataccgactacgccgtgtccgtgaagaatcggatcaccattaaccccgacacctcgaagaaccagttctcactccaactgaacagcgtgacccccgaggataccgcggtgtactactgcgcacaagaagtggaaccgcaggacgccttcgacatttggggacagggaacgatggtcacagtgtcgtccggtggaggaggttccggaggcggtggatctggaggcggaggttcggatatccagatgacccagagcccctcctcggtgtccgcatccgtgggcgataaggtcaccattacctgtagagcgtcccaggacgtgtccggatggctggcctggtaccagcagaagccaggcttggctcctcaactgctgatcttcggcgccagcactcttcagggggaagtgccatcacgcttctccggatccggttccggcaccgacttcaccctgaccatcagcagcctccagcctgaggacttcgccacttactactgccaacaggccaagtacttcccctataccttcggaagaggcactaagctggaaatcaaggcggccgcaaccactacaccagctccgcggccacccaccccagcaccaacaatagccagtcagcctttgtctctgagacctgaggcttgtcgacccgctgcaggtggggcagttcatactcggggtcttgatttcgcctgcgatatatatatttgggcccccctggcgggcacgtgtggggtgctccttctttcactcgtaattactctttactgtagggttaagttctcacgatccgccgatgcgccagcataccaacagggacagaaccaactttataatgagctgaatcttggtcgcagggaagaatatgatgtacttgataaacgcagaggccgggatcccgagatgggagggaaacctcggagaaagaacccccaggagggcctgtataatgaattgcaaaaagataaaatggctgaagcttattcagagattggaatgaaaggcgagcggagaagaggaaaagggcacgacgggctttaccaaggactgtccaccgcgacaaaggacacgtacgacgcccttcatatgcaggcgcttcctccacga

SEQ ID NO: 126 - protein, 450 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYTDYAVSVKNRITINPDTSKNQFSLQLNSVTPEDTAVYYCAQEVEPQDAFDIWGQGTMVTVSSGGGGSGGGGSGGGGSDIQMTQSPSSVSASVGDKVTITCRASQDVSGWLAWYQQKPGLAPQLLIFGASTLQGEVPSRFSGSGSGTDFTLTISSLQPEDFATYYCQQAKYFPYTFGRGTKLEIKAAATTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0367291 A1
(43) Pub. Date: Dec. 4, 2025

(54) Compositions and Methods for Treating Cancer with DuoCARs

(71) Applicant: Lentigen Technology, Inc., Gaithersburg, MD (US)

(72) Inventors: Rimas J. Orentas, Seattle, WA (US); Dina Schneider, Potomac, MD (US); Waleed M. Haso, Santa Monica, CA (US); Stefan Miltenyi, Bergish Gladbach (DE); Boro Dropulic, Ellicott City, MD (US)

(21) Appl. No.: 19/240,490

(22) Filed: Jun. 17, 2025

Related U.S. Application Data
(63) Continuation of application No. 17/540,377, filed on Dec. 2, 2021, now Pat. No. 12,365,718.
Application No. 17/540,377 is a continuation of application No. 16/692,957, filed on Nov. 22, 2019, now Pat. No. 11,208,455.
Application No. 16/692,957 is a continuation-in-part of application No. PCT/US2019/051734, filed on Sep. 18, 2019.
Application No. PCT/US2019/051734 is a continuation of application No. 16/134,735, filed on Sep. 18, 2018, now Pat. No. 10,689,431.
Application No. 16/134,735 is a continuation-in-part of application No. 16/078,269, filed on Aug. 21, 2018, now Pat. No. 11,453,712.
(60) Provisional application No. 62/382,791, filed on Sep. 2, 2016.

(51) Int. Cl. A61K 40/31; A61K 35/17; A61K 40/11; A61K 40/15; A61K 40/42; A61K 40/50; A61P 35/02; C07K 14/725

(52) U.S. Cl. CPC A61K 40/31; A61K 35/17; A61K 40/11; A61K 40/15; A61K 40/42; A61K 40/50; A61P 35/02; C07K 14/7051; A61K 2239/48

(57) ABSTRACT

Novel therapeutic immunotherapy compositions comprising at least two vectors, each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs are provided herein as well as are methods of use of same in a patient-specific immunotherapy that can be used to treat cancers and other diseases and conditions.

### CROSS-REFERENCE TO RELATED APPLICATIONS

This U.S. Utility patent application claims priority to PCT Application No. PCT/US19/51734, filed Sep. 18, 2019, which in turn claims priority to U.S. Utility patent application Ser. No. 16/134,735, filed on Sep. 18, 2018, which is a continuation-in-part application of U.S. Utility patent application Ser. No. 16/078,269, filed Aug. 21, 2018, which in turn claims priority to PCT Application No. PCT/US17/49923, filed Sep. 1, 2017, which in turn claims the benefit of priority under 35 U.S.C. Section 119 (e) to U.S. Provisional Patent Application No. 62/382,791 filed on Sep. 2, 2016, the entire contents of each of which are incorporated herein by reference.

### FIELD OF THE DISCLOSURE

This application relates to the field of cancer, particularly to a composition comprising at least two vectors encoding functional chimeric antigen receptors and methods of use of same in patient-specific immunotherapy.

### BACKGROUND OF THE INVENTION

Cancer is one of the deadliest threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult. One of the difficulties in modern cancer treatments is the amount of time that elapses between a biopsy and the diagnosis of cancer, and effective treatment of the patient. During this time, a patient's tumor may grow unimpeded, such that the disease has progressed further before treatment is applied. This negatively affects the prognosis and outcome of the cancer.

Chimeric Antigen Receptors (DuoCARs) are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4): e23621). The antigen-binding motif of a CAR is commonly fashioned after a single chain Fragment variable (scFv), the minimal binding domain of an immunoglobulin (Ig) molecule. Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or gamma-delta T cells) are also under development (Brown C E et al. Clin Cancer Res. 2012; 18 (8): 2199-209; Lehner M et al. PLOS One. 2012; 7 (2): e31210). There remains significant work with regard to defining the most active T-cell population to transduce with CAR vectors, determining the optimal culture and expansion techniques, and defining the molecular details of the CAR protein structure itself.

The linking motifs of a CAR can be a relatively stable structural domain, such as the constant domain of IgG, or designed to be an extended flexible linker. Structural motifs, such as those derived from IgG constant domains, can be used to extend the scFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ζ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well (Zhao Y et al. J Immunol. 2009; 183 (9): 5563-74). With the advent of new technology, the activation of T cells with beads linked to anti-CD3 and anti-CD28 antibody, the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia (Haso W, Lee D W, Shah N N, Stetler-Stevenson M, Yuan C M, Pastan I H, Dimitrov D S, Morgan R A, FitzGerald D J, Barrett D M, Wayne A S, Mackall C L, Orentas R J. Anti-CD22-chimeric antigen receptors targeting B cell precursor acute lymphoblastic leukemia. Blood. 2013; 121 (7): 1165-74; Kochenderfer J N et al. Blood. 2012; 119 (12): 2709-20). This is borne out by the clinical success of CD19-specific CARs that are in a second generation CD28/CD3-ζ (Lee D W et al. American Society of Hematology Annual Meeting. New Orleans, LA; Dec. 7-10, 2013) and a CD137/CD3-ζ signaling format (Porter D L et al. N Engl J Med. 2011; 365 (8): 725-33). In addition to CD137, other tumor necrosis factor receptor superfamily members such as OX40 also are able to provide important persistence signals in CAR-transduced T cells (Yvon E et al. Clin Cancer Res. 2009; 15 (18): 5852-60). Equally important are the culture conditions under which the CAR T-cell populations were cultured.

Current challenges in the more widespread and effective adaptation of CAR therapy for cancer relate to a paucity of compelling targets. Creating binders to cell surface antigens is now readily achievable, but discovering a cell surface antigen that is specific for tumor while sparing normal tissues remains a formidable challenge. One potential way to imbue greater target cell specificity to CAR-expressing T cells is to use combinatorial CAR approaches. In one system, the CD3-ζ and CD28 signal units are split between two different CAR constructs expressed in the same cell; in another, two DuoCARs are expressed in the same T cell, but one has a lower affinity and thus requires the alternate CAR to be engaged first for full activity of the second (Lanitis E et al. Cancer Immunol Res. 2013; 1 (1): 43-53; Kloss C C et al. Nat Biotechnol. 2013; 31 (1): 71-5). A second challenge for the generation of a single scFv-based CAR as an immunotherapeutic agent is tumor cell heterogeneity. At least one group has developed a CAR strategy for glioblastoma whereby the effector cell population targets multiple antigens (HER2, IL-13Ra, EphA2) at the same time in the hope of avoiding the outgrowth of target antigen-negative populations (Hegde M et al. Mol Ther. 2013; 21 (11): 2087-101).

T-cell-based immunotherapy has become a new frontier in synthetic biology; multiple promoters and gene products are envisioned to steer these highly potent cells to the tumor microenvironment, where T cells can both evade negative regulatory signals and mediate effective tumor killing. The elimination of unwanted T cells through the drug-induced dimerization of inducible caspase 9 constructs with AP1903 demonstrates one way in which a powerful switch that can control T-cell populations can be initiated pharmacologically (Di Stasi A et al. N Engl J Med. 2011; 365 (18): 1673-83). The creation of effector T-cell populations that are immune to the negative regulatory effects of transforming growth factor-β by the expression of a decoy receptor further demonstrates that degree to which effector T cells can be engineered for optimal antitumor activity (Foster A E et al. J Immunother. 2008; 31 (5): 500-5).

Thus, while it appears that CARs can trigger T-cell activation in a manner similar to an endogenous T-cell receptor, a major impediment to the clinical application of CAR-based technology to date has been limited in vivo expansion of CAR+ T cells, rapid disappearance of the cells after infusion, disappointing clinical activity, relapse of the underlying medical disease or condition, and the undue length of time that elapses between diagnosis and timely treatment of cancer using such CAR+ T cells.

Accordingly, there is an urgent and long felt need in the art for discovering compositions and methods for treatment of cancer using a CAR-based therapy that can exhibit cancer-specific intended therapeutic attributes without the aforementioned short comings.

The present invention addresses these needs by providing compositions comprising at least two vectors encoding functional chimeric antigen receptors and methods of use of same in patient-specific immunotherapy that can be used to treat cancers and other diseases and/or conditions.

In particular, the present invention as disclosed and described herein provides an immunotherapy composition comprising one or more isolated nucleic acid molecules encoding at least two vectors, each vector encoding a functional DuoCAR, whereby the combination of vectors results in the expression of two or more non-identical binding domains, wherein each vector encoded binding domain(s) are covalently linked to a transmembrane domain and one or more non-identical intracellular signaling motifs, which immunotherapy composition may be used to transduce autologous lymphocytes to generate active patient-specific anti-tumor lymphocyte cell populations that can be infused directly back into the patient to promote in vivo expansion, persistence of patient-specific anti-tumor T-cells resulting in tumor stabilization, reduction, elimination, remission of cancer, or prevention or amelioration of relapse of cancer, or a combination thereof, in a patient-specific manner.

### SUMMARY OF THE INVENTION

Novel adoptive immunotherapy compositions comprising two or more vector-transduced lymphocytes are provided herein as well as are methods of use of same in a patient-specific combination immunotherapy that can be used to treat cancers and other diseases and conditions.

Thus, in one aspect, lentiviral vectors expressing Duo chimeric antigen receptors (DuoCARs) are provided herein, as well as nucleic acid molecules encoding the lentiviral vectors expressing DuoCARs. Methods of using the disclosed lentiviral vectors expressing DuoCARs, host cells, and nucleic acid molecules are also provided, for example, to treat a cancer in a subject.

In one aspect, an immunotherapy composition is provided comprising one or more isolated nucleic acid molecules encoding at least two vectors (DuoCARs), each vector encoding a functional CAR, wherein at least one binding domain(s) in one of the vectors are non-identical, and whereby the combination of vectors results in the expression of two or more non-identical binding domains, wherein each vector encoded binding domain(s) are covalently linked to a transmembrane domain and one or more non-identical intracellular signaling motifs.

In one embodiment, an immunotherapy composition is provided comprising one or more isolated nucleic acid molecules encoding at least three vectors (TrioCARs), each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs.

In one embodiment, an immunotherapy composition is provided comprising one or more isolated nucleic acid molecules encoding at least four vectors (QuatroCARs), each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs.

In yet another embodiment, an immunotherapy composition is provided comprising one or more isolated nucleic acid molecules encoding at least two, three, four, five, six, seven, eight, nine, or ten vectors (e.g., an “nCAR”), each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs, wherein each unique member of the nCAR set when assembled into a CAR product constitutes a unique CAR composition referred to herein as “nCAR” (e.g., DuoCAR, TrioCAR, QuatroCAR, PentaCAR, HexaCAR, HeptaCAR, OctaCAR, NonaCAR, and DecaCAR, etc.).

In one embodiment, an immunotherapy composition is provided comprising: (a) at least two vectors, each comprising nucleic acid sequences that are functional in cells; (b) wherein each vector encodes a functional CAR; (c) wherein each CAR comprises of at least one binding domain, a single transmembrane domain, and at least one intracellular signaling motif; (d) wherein the at least one binding domains in one of the vectors are non-identical; and (e) wherein the at least one binding domain, a single transmembrane domain, at least one linker domain, and at least one intracellular signaling motif are covalently linked in each said vector, wherein the combination of vectors are used to genetically modify one or more lymphocyte populations.

In another embodiment, an immunotherapy composition is provided comprising: (a) at least two vectors, each comprising nucleic acid sequences that are functional in cells; (b) wherein each vector encodes a functional CAR; (c) wherein each CAR comprises at least one binding domain, a single transmembrane domain, and at least one intracellular signaling motif; (d) wherein the at least one binding domain(s) in each vector are non-identical; (e) wherein the at least one signaling motif combinations are non-identical between each of the vectors; and (f) wherein the at least one binding domain, a single transmembrane domain, and at least one intracellular signaling motif are covalently linked in each said vector, wherein the combination of two or more vectors are used to genetically modify one or more lymphocyte populations.

In one embodiment, an immunotherapy composition is provided wherein each vector encodes more than one functional CAR.

In another embodiment, an immunotherapy composition is provided wherein one or more signaling motifs combinations are identical on one or more vectors.

In another embodiment, an immunotherapy composition is provided wherein one or more multiple binding domains are identical on one or more vectors.

In another embodiment, an immunotherapy composition is provided wherein the lymphocyte population(s) comprise autologous T-cells or a mixture of peripheral blood derived lymphocytes.

In another embodiment, an immunotherapy composition is provided wherein the at least one extracellular antigen binding domain of the CAR comprises at least one single chain variable fragment of an antibody that binds to the antigen.

In another embodiment, an immunotherapy composition is provided wherein the at least one extracellular antigen binding domain of the CAR comprises at least one heavy chain variable region of an antibody that binds to the antigen.

In another embodiment, an immunotherapy composition is provided wherein the at least one extracellular antigen binding domain of the CAR, the at least one intracellular signaling domain of the CAR, or both are connected to the transmembrane domain by a linker or spacer domain.

In another embodiment, an immunotherapy composition is provided wherein the extracellular antigen binding domain of the CAR is preceded by a leader peptide.

In another embodiment, an immunotherapy composition is provided wherein the extracellular antigen binding domain of the CAR targets an antigen comprising CD19, CD20, CD22, ROR1, TSLPR, mesothelin, CD33, CD38, CD123 (IL3RA), CD138, BCMA (CD269), GPC2, GPC3, FGFR4, c-Met, PSMA, Glycolipid F77, EGFRVIII, GD-2, NY-ESO-1, MAGE-A3, PRAME peptides in combination with MHC, or any combination thereof.

In another embodiment, an immunotherapy composition is provided wherein the extracellular antigen binding domain of the CAR comprises an anti-CD19 scFV antigen binding domain, an anti-CD20 scFV antigen binding domain, an anti-CD22 scFV antigen binding domain, an anti-RORI scFV antigen binding domain, an anti-TSLPR scFV antigen binding domain, an anti-mesothelin scFV antigen binding domain, an anti-CD33 scFV antigen binding domain, an anti-CD38 scFV antigen binding domain, an anti-CD123 (IL3RA) scFV antigen binding domain, an anti-CD138 scFV antigen binding domain, an anti-BCMA (CD269) scFV antigen binding domain, an anti-GPC2 scFV antigen binding domain, an anti-GPC3 scFV antigen binding domain, an anti-FGFR4 scFV antigen binding domain, an anti-c-Met scFV antigen binding domain, an anti-PMSA scFV antigen binding domain, an anti-glycolipid F77 scFV antigen binding domain, an anti-EGFRvIII scFV antigen binding domain, an anti-GD-2 scFV antigen binding domain, an anti-NY-ESO-1 TCR (including single chain TCR constructs) antigen binding domain, an anti-MAGE-A3 TCR, or an amino acid sequence with 85%, 90%, 95%, 96%, 97%, 98% or 99% identity thereof, or any combination thereof.

In another embodiment, an immunotherapy composition is provided wherein the linker or spacer domain of the CAR is derived from the extracellular domain of CD8, and is linked to the transmembrane domain.

In another embodiment, an immunotherapy composition is provided wherein the CAR further comprises a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19, Fc epsilon R, or any combination thereof.

In another embodiment, an immunotherapy composition is provided wherein the at least one intracellular signaling domain further comprises a CD3 zeta intracellular domain.

In another embodiment, an immunotherapy composition is provided wherein the at least one intracellular signaling domain is arranged on a C-terminal side relative to the CD3 zeta intracellular domain.

In another embodiment, an immunotherapy composition is provided wherein the at least one intracellular signaling domain comprises a costimulatory domain, a primary signaling domain, or any combination thereof.

In another embodiment, an immunotherapy composition is provided wherein the at least one costimulatory domain comprises a functional signaling domain of OX40, CD70, CD27, CD28, CD5, ICAM-1, LFA-1 (CD11a/CD18), ICOS (CD278), DAP10, DAP12, and 4-1BB (CD137), PD-1, GITR, CTLA-4, or any combination thereof.

In another embodiment, an immunotherapy composition is provided wherein a single vector is used to encode all chimeric antigen receptors (e.g., retroviral, adenoviral, SV40, herpes vector, POX vector, RNA, plasmid, cosmid, or any viral vector or non-viral vector), in combination with a CRISPR system for integration.

In another embodiment, an immunotherapy composition is provided wherein each vector is an RNA or DNA vector, alone or in combination with a transfection reagent or a method to deliver the RNA or DNA into the cell, a non-limiting example being electroporation.

In another embodiment, an immunotherapy composition is provided wherein at least one vector expresses a nucleic acid molecule that modulates the expression of a nucleic acid in the cell.

In another embodiment, an immunotherapy composition is provided wherein the nucleic acid molecule inhibits or deletes the expression of an endogenous gene.

In certain embodiments, an immunotherapy composition is provided wherein the active patient-specific autologous anti-tumor lymphocyte cell population is generated within one day, two days, three days, four days, five days, seven days, ten days, twelve days, fourteen days, twenty-one days, or one month of lymphocyte harvest or tumor biopsy and wherein the active patient-specific autologous anti-tumor lymphocyte cell population that can be infused back into a patient suffering from cancer and is capable of promoting in vivo expansion, persistence of patient-specific anti-tumor lymphocyte cells resulting in tumor stabilization, reduction, elimination, remission of cancer, or prevention or amelioration of relapse of cancer, or a combination thereof, in a patient-specific manner.

In one aspect, isolated nucleic acid molecules encoding the aforementioned chimeric antigen receptors are provided herein.

In one aspect of the DuoCARs used in the patient-specific autologous lymphocyte population(s) of the immunotherapy composition of the present invention, the DuoCARs are modified to express or contain a detectable marker for use in diagnosis, monitoring, and/or predicting the treatment outcome such as progression free survival of cancer patients or for monitoring the progress of such treatment. In one embodiment of the DuoCARs used in the patient-specific autologous anti-tumor lymphocyte cell population(s), the nucleic acid molecules encoding the disclosed DuoCARs can be contained in a vector, such as a viral or non-viral vector. The vector is a DNA vector, an RNA vector, a plasmid vector, a cosmid vector, a herpes virus vector, a measles virus vector, a lentiviral vector, adenoviral vector, or a retrovirus vector, or a combination thereof.

In certain embodiments of the DuoCARs used in the patient-specific autologous anti-tumor lymphocyte cell population(s), the two or more lentiviral vectors are pseudotyped with different viral glycoproteins (GPS) including for example, and not by way of limitation, amphotropic murine leukemia virus [MLV-A], a baboon endogenous virus (BaEV), GP164, gibbon ape leukemia virus [GALV], RD114, feline endogenous virus retroviral-derived GPs, and vesicular stomatitis virus [VSV], measles virus, fowl plague virus [FPV], Ebola virus [EboV], lymphocytic choriomeningitis virus [LCMV]) non retroviral-derived GPs, as well as chimeric variants thereof including, for example, and not by way of limitation, chimeric GPs encoding the extracellular and transmembrane domains of GALV or RD114 GPs fused to the cytoplasmic tail (designated TR) of MLV-A GP.

In certain embodiments of the DuoCARs used in the patient-specific autologous anti-tumor lymphocyte cell population(s), the vector further comprises a promoter wherein the promoter is an inducible promoter, a tissue specific promoter, a constitutive promoter, a suicide promoter or any combination thereof.

In yet another embodiment of the DuoCARs used in the patient-specific autologous anti-tumor lymphocyte cell population(s), the vector expressing the CAR can be further modified to include one or more operative elements to control the expression of CAR T cells, or to eliminate CAR-T cells by virtue of a suicide switch. The suicide switch can include, for example, an apoptosis inducing signaling cascade or a drug that induces cell death. In a preferred embodiment, the vector expressing the CAR can be further modified to express an enzyme such thymidine kinase (TK) or cytosine deaminase (CD).

In another aspect of the DuoCARs used in the patient-specific autologous anti-tumor lymphocyte cell population(s), host cells including the nucleic acid molecule(s) encoding the DuoCARs are also provided. In some embodiments, the host cell is a T cell, such as a primary T cell obtained from a subject. In one embodiment, the host cell is a CD8+ T cell. In one embodiment the host cell is a CD4+ T cell. In one embodiment the host cells are selected CD4+ and CD8+ lymphocytes purified directly from a patient product without regard to proportionality. In another embodiment the number of CD4+ and CD8+ T cells in the product are specific. In another embodiment specific subsets of T cells are utilized as identified by phenotypic markers including T naïve cells (Tn), T effector memory cells (Tem), T central memory cells (Tcm), T regulatory cells (Treg), induced T regulatory cells (iTreg), T suppressor cells (Ts), T stem cell memory cells (Tscm), Natural Killer (NK) cells, and lymphokine activated killer (LAK) cells.

In yet another embodiment, a pharmaceutical composition is provided comprising an anti-tumor effective amount of an immunotherapy composition comprising a population of patient-specific autologous anti-tumor lymphocyte cell population(s) of a human having a cancer, wherein the cells of the population include cells comprising nucleic acid molecules encoding at least two vectors, each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs.

In yet another embodiment, a pharmaceutical composition is provided comprising an anti-tumor effective amount of an immunotherapy composition comprising a population of patient-specific autologous anti-tumor lymphocyte cell population(s) of a human having a cancer, wherein the cells of the population include cells comprising (a) nucleic acid molecules encoding two or more vectors; (b) wherein each vector encodes a functional CAR; (c) wherein each CAR comprises of at least one binding domain, at least one transmembrane domain, at least one linker domain, and at least one intracellular signaling motif; (d) wherein the at least one binding domains in one of the vectors are non-identical; and (e) wherein the at least one binding domain, a single transmembrane domain, at least one linker domain, and at least one intracellular signaling motif are covalently linked in each said vector, wherein the combination of vectors are used to genetically modify one or more lymphocyte populations.

In yet another embodiment, a pharmaceutical composition is provided comprising an anti-tumor effective amount of an immunotherapy composition comprising a population of patient-specific autologous anti-tumor lymphocyte cell population(s) of a human having a cancer, wherein the cells of the population include cells comprising (a) nucleic acid molecules encoding two or more vectors; (b) wherein each vector encodes a functional CAR; (c) wherein each CAR comprises at least one binding domain, at least one transmembrane domain, at least one linker domain, and at least one intracellular signaling motif; (d) wherein the at least one binding domain(s) in each vector are non-identical; (e) wherein the at least one signaling motif combinations are non-identical between each of the vectors; and (f) wherein the at least one binding domain, a single transmembrane domain, at least one linker domain, and at least one intracellular signaling motif are covalently linked in each said vector, wherein the combination of two or more vectors are used to genetically modify one or more lymphocyte populations.

In one embodiment, the cancer is a refractory cancer non-responsive to one or more chemotherapeutic agents. The cancer includes hematopoietic cancer, myelodysplastic syndrome, pancreatic cancer, head and neck cancer, cutaneous tumors, minimal residual disease (MRD) in acute lymphoblastic leukemia (ALL), acute myeloid leukemia (AML), lung cancer, breast cancer, ovarian cancer, prostate cancer, colon cancer, melanoma or other hematological cancer and solid tumors, or any combination thereof. In another embodiment, the cancer includes a hematological cancer such as leukemia (e.g., chronic lymphocytic leukemia (CLL), acute lymphocytic leukemia (ALL), acute myeloid leukemia (AML), or chronic myelogenous leukemia (CML), lymphoma (e.g., mantle cell lymphoma, non-Hodgkin's lymphoma or Hodgkin's lymphoma) or multiple myeloma, or any combination thereof.

In yet another embodiment, the cancer includes an adult carcinoma comprising coral and pharynx cancer (tongue, mouth, pharynx, head and neck), digestive system cancers (esophagus, stomach, small intestine, colon, rectum, anus, liver, intrahepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

In another aspect, a pharmaceutical composition is provided comprising an autologous lymphocyte cell population transduced with two or more lentiviral vectors encoding single or multiple chimeric antigen receptors (DuoCARs), thereby generating a patient-specific autologous anti-tumor lymphocyte cell population capable of promoting in vivo expansion, persistence of patient-specific anti-tumor T-cells resulting in tumor stabilization, reduction, elimination, remission of cancer, or prevention or amelioration of relapse of cancer, or a combination thereof, in a patient-specific manner.

In another aspect, a pharmaceutical composition is provided comprising an autologous T cell population transduced with one or more lentiviral vectors encoding single or multiple chimeric antigen receptors (DuoCARs) to generate an patient-specific autologous anti-tumor lymphocyte cell population capable of promoting in vivo expansion, persistence of patient-specific anti-tumor T-cells resulting in tumor stabilization, reduction, elimination, remission of cancer, or prevention or amelioration of relapse of cancer, or a combination thereof, in a patient-specific manner.

In another aspect, methods of making active patient-specific autologous anti-tumor Duo CAR-containing lymphocyte cells are provided. The methods include transducing a lymphocyte cell with two or more vectors or nucleic acid molecule encoding two or more chimeric antigen receptors (DuoCARs) that specifically bind an antigen, thereby making active patient-specific autologous anti-tumor Duo CAR-containing lymphocyte cells.

In yet another aspect, a method of generating a population of RNA-engineered lymphocyte cells is provided that comprises introducing an in vitro transcribed RNA or synthetic RNA of a nucleic acid molecule encoding a two or more chimeric antigen receptors (DuoCARs) into a cell population of a subject, thereby generating an patient-specific autologous anti-tumor lymphocyte cell population capable of promoting in vivo expansion, persistence of patient-specific anti-tumor T-cells resulting in tumor stabilization, reduction, elimination, remission of cancer, or prevention or amelioration of relapse of cancer, or a combination thereof, in a patient-specific manner.

In another aspect, a method is provided for treating a mammal having a disease, disorder or condition associated with an elevated expression of a tumor antigen, the method comprising administering to the subject a pharmaceutical composition comprising an anti-tumor effective amount of an autologous lymphocyte cell population transduced with one or more lentiviral vectors encoding single or multiple chimeric antigen receptors (DuoCARs) thereby generating an patient-specific autologous anti-tumor lymphocyte cell population capable of promoting in vivo expansion, persistence of patient-specific anti-tumor T-cells resulting in tumor stabilization, reduction, elimination, remission of cancer, or prevention or amelioration of relapse of cancer, or a combination thereof, in a patient-specific manner.

In another aspect, a method is provided for treating a mammal having a disease, disorder or condition associated with an elevated expression of a tumor antigen, the method comprising administering to the subject a pharmaceutical composition comprising an anti-tumor effective amount of an autologous lymphocyte cell population transduced with two or more lentiviral vectors encoding single or multiple chimeric antigen receptors (DuoCARs) to generate an patient-specific autologous anti-tumor lymphocyte cell population which can be infused directly back into the patient to promote in vivo expansion, persistence of patient-specific anti-tumor T-cells resulting in tumor stabilization, reduction, elimination, or remission of cancer, or prevention or amelioration of relapse of cancer, or any combination thereof, in a patient-specific manner.

In one embodiment, a method is provided for treating a mammal having a disease, disorder or condition associated with an elevated expression of a tumor antigen, the method comprising administering to the subject a pharmaceutical composition comprising at least two vectors, each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs, and a pharmaceutically acceptable excipient, wherein the combination of vectors are used to genetically modify one or more lymphocyte populations.

In another embodiment, a method is provided for treating a mammal having a disease, disorder or condition associated with an elevated expression of a tumor antigen, the method comprising administering to the subject a pharmaceutical composition comprising (a) nucleic acid molecules encoding two or more vectors; (b) wherein each vector encodes a functional CAR; (c) wherein each CAR comprises of at least one binding domain, at least one transmembrane domain, and at least one intracellular signaling motif; (d) wherein the at least one binding domains in one of the vectors are non-identical; and (e) wherein the at least one binding domain, a single transmembrane domain, and at least one intracellular signaling motif are covalently linked in each said vector, wherein the combination of vectors are used to genetically modify one or more lymphocyte populations.

In yet another embodiment, a method is provided for treating a mammal having a disease, disorder or condition associated with an elevated expression of a tumor antigen, the method comprising administering to the subject a pharmaceutical composition comprising (a) nucleic acid molecules encoding two or more vectors; (b) wherein each vector encodes a functional CAR; (c) wherein each CAR comprises at least one binding domain, at least one transmembrane domain, and at least one intracellular signaling motif; (d) wherein the at least one binding domain(s) in each vector are non-identical; (e) wherein the at least one signaling motif combinations are non-identical between each of the vectors; and (f) wherein the at least one binding domain, a single transmembrane domain, and at least one intracellular signaling motif are covalently linked in each said vector, wherein the combination of two or more vectors are used to genetically modify one or more lymphocyte populations.

In certain embodiments, the genetically modified lymphocytes are autologous T cell lymphocytes, and wherein the autologous or allogeneic T cell lymphocytes are infused directly back into the patient so as to prevent or ameliorate relapse of malignant disease.

In certain other embodiments, the genetically modified lymphocytes are autologous T cell lymphocytes, and wherein the autologous lymphocytes are infused directly back into the patient to promote in vivo expansion, persistence of patient-specific anti-tumor T-cell lymphocytes resulting in tumor stabilization, reduction, elimination, or remission of cancer, or prevention or amelioration of relapse of cancer, or any combination thereof, in a patient-specific manner.

In yet another embodiment, the T cell has been preselected by virtue of expressing specific activation or memory-associated surface markers.

In yet another embodiment, the T cell is derived from a hematopoietic stem cell donor, and wherein the procedure is carried out in the context of hematopoietic stem cell transplantation.

In certain embodiments, a method is provided wherein the lymphocyte cell has been preselected by virtue of expressing specific activation or memory-associated surface markers.

In certain embodiments, a method is provided herein wherein the lymphocyte cell is a T cell and is derived from a hematopoietic stem cell donor, and wherein the procedure is carried out in the context of hematopoietic stem cell transplantation.

In yet another aspect, a method is provided for generating a persisting population of genetically engineered patient-specific autologous anti-tumor lymphocyte cell population(s) in a human diagnosed with cancer. In one embodiment, the method comprises administering to a human patient in need thereof one or more patient-specific autologous anti-tumor lymphocyte cell population(s) described herein, wherein the persisting population of patient-specific autologous anti-tumor lymphocyte cell population(s), or the population of progeny of the lymphocyte cells, persists in the human for at least one month, two months, three months, four months, five months, six months, seven months, eight months, nine months, ten months, eleven months, twelve months, two years, or three years after administration.

In one embodiment, the progeny lymphocyte cells in the human comprise a memory T cell. In another embodiment, the T cell is an autologous T cell.

In all of the aspects and embodiments of methods described herein, any of the aforementioned cancers, diseases, disorders or conditions associated with an elevated expression of a tumor antigen that may be treated or prevented or ameliorated using a patient-specific autologous anti-tumor lymphocyte cell population(s) comprising one or more of the Duo Car immunotherapeutic compositions as disclosed herein.

In yet another aspect, a kit is provided for making a DuoCAR immunotherapeutic composition comprising a patient-specific autologous anti-tumor lymphocyte cell population(s) as described supra or for preventing, treating, or ameliorating any of the cancers, diseases, disorders or conditions associated with an elevated expression of a tumor antigen in a subject as described supra, comprising a container comprising any one of the nucleic acid molecules, vectors, host cells, or compositions disclosed supra or any combination thereof, and instructions for using the kit.

While the compositions and methods of the present invention have been illustrated with reference to the generation and utilization of DuoCARs, it is contemplated herein that the compositions and methods are specifically intended to include the generation and utilization of TrioCARs and QuatroCARs.

In yet another aspect, an immunotherapy composition comprising one or more isolated nucleic acids encoding at least one vector, wherein said vector contains a nucleic acid sequence that results in at least one messenger RNA (i.e., a multi-cistronic nucleic acid or a nucleic acid resulting in more than one transcript) encoding a DuoCAR, resulting in the ability to bind two or more non-identical antigen targets, thereby generating multiple antigen specificities residing in a single cell expressing said vector.

In yet another aspect, an immunotherapy composition comprising one or more isolated nucleic acids encoding at least two vectors, as described supra, wherein each vector further encodes a functional tag or anti-tag binding moiety (AT-CAR) that reconstitutes a functional chimeric antigen receptor upon co-incubation or co-administration of a soluble binder (such as a tagged scFv, or a scFv linked to an anti-tag binder), whereby the combination of the two vectors results in the ability to bind two or more non-identical antigen binding domains, resulting in multiple antigen specificities residing in a cell expressing these two vectors.

In yet another aspect, an immunotherapy composition comprising one or more isolated nucleic acids encoding at least two vectors, as described supra, wherein each vector encoding a functional tag or anti-tag binding moiety (AT-CAR) that reconstitutes a functional chimeric antigen receptor upon co-incubation or co-administration of a soluble binder (such as a tagged scFv, or a scFv linked to an anti-tag binder), wherein each vector expresses a unique tag (or anti-tag) that can bind soluble protein or protein modified structures resulting in multiple antigen specificities, or wherein each vector expresses a unique tag (or anti-tag) that binds only one of the soluble binding domains resulting in a specific linkage of the AT-CAR encoded intracellular signaling motifs to the antigen-binding domains of the tagged (or anti-tagged) binder.

In a non-limiting embodiment for the manufacture of DuoCAR vectors, the each of the compositions and methods disclosed in the embodiments and aspects referred to supra, the two vectors can be made separately and then added to the T cells sequentially or at the same time. In another non limiting embodiment, the plasmid DNA of the two or more vectors can be combined before or during transfection of production cells, or integrated in the production cells genome, to produce a mixture of viral vectors that contain the multiple DuoCAR vector particles, subsequently used for the transduction and genetic modification of patient T Cells.

For each of the various aspects and embodiments of the DuoCARs, TrioCARs and QuatroCARs specifically contemplated herein, the nucleotide sequences encoding the functional CAR comprise the nucleotide sequence of SEQ ID NO: 3, 9, 21, 25, 29, 31, 35, 39, 43, 47, 49, 51, 53, 55, 59, 61, 109, 111, 113, or 115 or any combination thereof.

For each of the various aspects and embodiments of the DuoCARs, TrioCARs and QuatroCARs specifically contemplated herein, each vector encodes a functional CAR comprising the amino acid sequence of SEQ ID NO: 4, 10, 22, 26, 30, 32, 36, 40, 44, 48, 50, 52, 54, 56, 60, 62, 110, 112, 114, or 116 or any combination thereof.

It will be understood that the patient-specific autologous anti-tumor lymphocyte cell population(s), the two or more lentiviral vectors expressing chimeric antigen receptors (DuoCARs), host cells, and methods as described supra are useful beyond the specific aspects and embodiments that are described in detail herein. The foregoing features and advantages of the disclosure will become more apparent from the following detailed description, which proceeds with reference to the accompanying figures.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts four (4) Products (Examples 1 through 4) that can be produced as discrete commercial entities. These DuoCARs sets can be created to target human B cell malignancies expressing three leukemia-associated antigens, CD19, CD20, and CD22. In Product 1, two gene vectors are used to co-transduce an activated T cell population. The first vector encodes two antigen binding domains (CD19, CD20) linked to a single intracellular domain (z, CD3 zeta chain) connected by virtue of a CD8 transmembrane region (8). The second vector encodes a CD22 binding domain and two signaling domains (BB, derived from CD137/4-1BB; and z). The second Product, Example 2, feature the first vector with CD19- and CD20-binding domains linked to CD28 and z signaling domains. The second vector encodes a CD22 binding domain and the BB and z signaling domains and essentially recapitulated the signaling package of a third generation CAR vector (three different signaling domains) In the third Product, Example 3, the first vector encodes CD20- and CD22-binding domain linked to BB and z signaling domains and the second vector encodes a CD19-binding domain linked to CD28 and z signaling domains. In the fourth Product, Example 4, the first vector encodes CD20- and CD22-binding domains and BB and z signaling domains. The second vector encodes a CD19 binding domains and a z signaling domain.

FIG. 2 depicts all potential single component that can be combined into DuoCARs for a therapeutic product targeting B cell malignancies. Nomenclature is identical to that in FIG. 1.

FIG. 3 depicts a generalized schema for DuoCARs that can be applied to multiple therapeutic needs, including inflammatory or autoimmune diseases and infectious diseases. In the Figure a-CDX, a-CDY, a-CDZ refer to antigen binding domains specific for three different target antigens, CDX, CDY, and CDZ, respectively. The intracellular aspect of the CARs all include the CD8 linker and transmembrane domain linked to either CD3-zeta, CD28, or 4-1BB signaling domains (as in FIG. 1). The specific combination of any of these two vectors (for example A plus F, wherein antigen X, Y, and Z would be targeted while providing intracellular signaling through CD3-zeta and 4-1BB) into a single vector will be defined according to the specific therapeutic need.

FIG. 4 depicts a generalized schema for DuoCAR sets in which two antigens are targeted by each vector. Vectors that are identical to those in FIG. 3 retain their specific letter designation (A in FIG. 3 and FIG. 4 are the same). The new, fourth, antigen binding domain is indicated by a-CDW. One product that would target 4 antigens be an A+T Duo CAR set. In this instance the extracellular antigens CDX, CDY, CDZ, and CDW would be targeted while providing both CD3-zeta and CD28 intracellular signals.

FIG. 5 depicts current CARs in the literature (A, B, C, D) in comparison to the DuoCARs of the present invention (E, F, G). CAR expression vectors can be created that induce expression of a single binding domain (paired black, open or striped spheres, each with separate specificities) connected to a linker and transmembrane domain (single open box). In the figure a thick gray line represents the plasma cell membrane. In this figure, the paired black spheres could represent anti-CD19 scFv, the paired open spheres represent anti-CD20 scFv and the paired striped spheres represent anti-CD22 scFv, all linked by joining amino acid sequences, for examples, multimers (1, 2, 3, 4, 5, or 6 repeats) of GGGGS. Intracellularly the lymphocyte signaling domains derived from 4-1BB (CD137), CD28, and the CD3-zeta chain can be combined as shown. (A) In Single CARs, a single binding domain is combined with a transmembrane and 2 signaling domains, created a second-generation CAR. (B) In Split CARS, two different binders are expressed with single signaling domains that must be combined to render effective T cell signaling upon recognition of two distinct antigens. (C) In Tandem CARs, two binding domains are linked to a single signaling domain. In this case binding of either domain induces full T cell activation. (D) In Multiple CARs from one vector, two fully functional CARs are expressed from a single vector, each able to bind only one antigen. (E) In contrast, DuoCARs are comprised of two vectors and express at least three binding domains, with multiple combinations of signaling domains possible. Essential features that differentiate the DuoCAR is the expression of two or more transcripts, the multiplicity of binding domains (at least one being multi-targeting), and the fully functional signaling characteristics of at least one of the two expressed cell surface proteins. (F) In a DuoCAR single-specificity soluble binder format, the CAR portion encoded by the vectors express a tag or an anti-tag motif that also encodes transmembrane and intracellular signaling motifs (CAR base vectors, non-identical with respect to intracellular motifs). The base vectors bind soluble proteins containing both the scFv domains that interact with antigen and a tag or anti-tag motif to mediate binding to the CAR base protein itself. Once the soluble proteins bind to the CAR base proteins, the same structural characteristics that mediate anti-tumor activity mediated by the DuoCAR [as in (E)] are reconstituted. (G) In a DuoCAR, dual-specificity soluble binder format, the dual specificity “tag”-“anti-tag” interactions are unique such that only one of the soluble binders can bind to only one of the base vectors. In this instance, the black diamond on the base vector and the angle-shaped binder on the soluble dual scFv protein may represent a “biotin”-“anti-biotin” interaction and the black crescent shape on the second CAR base vector interacts with the black oval on the single specificity scFv structure and may represent a “FITC”-“anti-FITC” interaction.

FIG. 6 depicts cell-surface expression levels of CAR constructs on primary human 5 T cells transduced with CAR expression vectors that differ between second generation (two costimulatory domains) and third generation (three costimulatory domains) formats. T cells were transduced to express the following CARs: no CAR (mock), a second generation CAR (CAR-A-28z), a third generation CAR (CAR-A-28BBz), and an alternate second generation CAR (CAR-A-BBz). The level of surface expression of the CAR was detected by flow cytometry and is reported as mean fluorescence intensity (MF), y-axis. The MFI of both second 20 generation CARs was much brighter, even though all construct expressed the very same CAR binding domain.

FIG. 7 depicts DuoCAR cell surface expression in human T cells. Human T cells were activated with CD3-CD28 nanomatrix (TransAct, Miltenyi Biotec) in the presence of IL-2, transduced with two vectors (one encoding a tandem CD20-CD19 CAR and one encoding a single CD22 CAR, thus a 2+1 Duo-Set format), and then analyzed for expression of CD19-, CD20-, or CD22-scFv domains by flow cytometry using recombinant CD19, CD20, or CD22 for staining. The paired columns show dual staining for CD20 and CD19 scFvs, left column, and CD22 and CD19 scFvs, right column. Row 1 shows T cells that were not transduced (UTD) and thus show no binding. Row 2 shows T cells transduced with LV encoding a CD20_CD19 CAR vector with a CD8 transmembrane and intracellular CD28 and CD3-zeta signaling domains (20-19-28z). While dual staining is seen for CD20 and CD19 binding (left panel), only CD19 binding is seen in the right panel. Row 3 shows T cells transduced with a CD22 CAR vector with a CD8 transmembrane and intracellular 4-1BB and CD3-zeta signaling domains (22-BBz). No dual staining is seen with CD19 or CD20 (left panel) and only a single population of cells able to bind CD22 is seen (right panel). In Row 4 T cells are transduced with a DuoSet comprised of both vectors in Row 2 and Row 3. Only the DuoSet express all three CAR-encoded binding domains (42% of the cells express CD20_19 (left panel), and 38% expresses CD22 and CD19 binding domains (right panel). As CD22 and CD19 scFv are on each of the two separate transmembrane proteins comprising the DuoSet, 38% represents the true DuoSet expressing population in this example.

FIG. 8 depicts the anti-tumor cytolytic activity of DuoCAR expressing T cells. Human T cells transduced with single CAR components (20_19-28z or 22-BBz) or DuoCARs (20_19-28z+22-BBz), as described in FIG. 7, were used in cytotoxic T cells assay at four different effector to target ratios (20:1, 10:1, 5:1, 2.5:1, as indicated). The leukemia cell lines used as CAR-T targets were: Raji (expresses all three target antigens), REH (expresses all three target antigens), K562 (control, no targets expressed), K562-CD19 (expresses CD19), K562-CD20 (expresses CD20), and K562-CD22 (expresses CD22). Only the DuoCAR-transduced cells (20-19-28z+22-BBz, 2+1 DuoSet) exhibited high cytolytic activity against both leukemia cell lines (Raji and REH), and all three single-expressing K562 target cells lines (K562-CD19, K562-CD20, K562-CD22).

FIG. 9 depicts DuoCAR cell surface expression in primary human T cells, as achieved by two different methods of LV preparation. The same methods and data analyses were used as in FIG. 7, thus cells transduced with a DuoCAR specific for CD19, CD20, and CD22 (a 2+1 DuoSet where one CAR is a tandem CD20 and CD19 binder and the second CAR is comprised of a CD22 binder) were created. The first column of data shows flow cytometric analysis for the expression of CD19 and CD20 binders, whereas the second column shows flow cytometric analysis for CD22 and CD19 binders present as CARs in DuoCAR expressing cells for four distinct populations corresponding to the non-transduced, the singly CD22-CAR transduced, the dually transduced with CD22 and CD20_19 CARs, and singly transduced with the tandem CD20_CD19 CAR in the lower left, upper left, upper right, and lower right quadrants, respectively. Both the two LV transduction method (co-transduction) and the single LV transduction method (co-transfection) gave a similar DuoCAR staining pattern, where more than 30% of the T cell population was specific for CD19, CD20, and CD22, by virtue of expressing both CAR cell surface proteins.

FIG. 10 depicts a schematic representation of DuoCAR bicistronic constructs. DuoCAR constructs are expressed from a single bicistronic open reading frame, containing sequences of two CAR chains separated by 2A peptide. One CAR is comprised of CD22 scFv, linked in frame to CD8 hinge and transmembrane domain, 4-1BB costimulatory domain and CD3 zeta activation domain. Another CAR is comprised of a tandem CD20 CD19 scFv-based targeting domain, followed by CD8 hinge and transmembrane domain, CD28 costimulatory domain and CD3 zeta activation domain.

FIG. 11 depicts cell surface expression of Set 1 Bicistronic DuoCARs on primary human T cells transduced with DuoCAR expression vectors and controls as measured by flow cytometry. T cells were transduced to express the following CARs: no CAR (UTD), construct number 2228 (2019 tandem CAR), construct numbers 2200, 2209, 2218, 2225, 2227 (CD22 CAR variants), construct numbers (2515, 2520, 2521 Bicistronic CARs containing one CAR chain targeted to CD22, and another tandem CAR chain targeted to CD20 and CD19 tumor antigens). In bipartite plots shown, the CAR 22 expression is shown on the Y axis, and CAR 19 expression, representing the tandem 2019 CAR chain, is shown on the X axis. Percentage positive cells is denoted in each quadrant. Data are representative of three transduction experiments in T cells from separate healthy donors.

FIG. 12 depicts cytokine response of Bicistronic DuoCARs set 1 co-incubated with Raji tumor cells. T cells were transduced to express the following CARs: no CAR (UTD), construct number 2228 (-2019 tandem CAR), construct number 2200 (−CD22 CAR), construct numbers 2515, 2520, 2521 DuoCAR T cells and controls were incubated with triple positive Raji cells overnight, then supernatants were harvested and analyzed by ELISA for IFNg, TNFa and IL-2. N=3, +/−SD. One experiment representing three separate experiments in T cells from separate donors is shown.

FIG. 13 depicts cell surface expression of Set 2 Bicistronic DuoCARs on primary human T cells transduced with DuoCAR expression vectors and controls as measured by flow cytometry. T cells were transduced to express the following CARs: no CAR (UTD), construct number 1497 (-2019 tandem CAR), construct number 2200 (−CD22 CAR), construct numbers D0043, D0044, D0046, D0047-Bicistronic CARs containing one CAR chain targeted to CD22, and another tandem CAR chain targeted to CD20 and CD19 tumor antigens. In bipartite plots shown, the CAR 22 expression is shown on the Y axis, and CAR 19 expression, representing the tandem 2019 CAR chain, is shown on the X axis. Percentages of positive cells are denoted in each quadrant. Data are representative of three transduction experiments in T cells from three separate healthy donors.

FIG. 14 depicts the anti-tumor cytolytic activity of Set 2 Bicistronic DuoCARs-expressing T cells. Human T cells transduced with single CAR components (LTG1497, 20_19-28z or LTG2200, 22-BBz) or DuoCARs (construct numbers D0043, D0044, D0046, D0047, encoding 20_19-28z+22-BBz), were used in cytotoxic T cells assay at four different effector to target ratios (10:1, 5:1, 2.5:1, as indicated, zeroes between “D” and the numerical designation in the construct name were omitted for simplicity). The leukemia cell lines used as CAR-T targets were: Raji (expresses all three target antigens), Reh (expresses all three target antigens), 392T (devoid of all three target antigens). DuoCARs lysed triple-positive cell lines in E:T dependent manner, and no lysis occurred in target negative 293T cell line.

FIG. 15 depicts the anti-tumor cytolytic activity of Bicistronic DuoCAR Set 2 expressing T cells. Human T cells transduced with single CAR components (LTG1497, 20_19-28z or LTG 2200, 22-BBz) or DuoCARs (construct numbers D0043, D0044, D0046, D0047, encoding 20_19-28z+22-BBz), were used in cytotoxic T cells assay at four different effector to target ratios (10:1, 5:1, 2.5:1. As indicated, zeroes between “D” and the numerical designation in the construct name were omitted for simplicity). The single-positive tumor cell lines used as CAR-T targets were: K19 (expresses CD19), K20 (expresses CD20), and K22 (expresses CD22). The three single-positive tumor cell lines were developed on the background of the parent K562 erythroleukemia line, which is naturally devoid of CD19, CD20 or CD22 expression, by stable transduction of the desired single antigen (CD19, CD20, or CD22) and the firefly luciferase gene. DuoCARs lysed single-positive cell lines in E:T dependent manner, and no lysis above background level was mediated by CAR controls with mismatched antigen targeting domains (CAR 22, LTG 2200 vs K19 and K20, tandem CAR 2019, LTG 1479 vs K22).

FIG. 16 depicts cytokine response of Bicistronic DuoCARs version 2 co-incubated with Raji tumor cells or incubated in the absence of tumors (CAR alone). T cells were transduced to express the following CARs: no CAR (UTD), construct number 2273 (-2019 tandem single chain CAR), construct number 2200 (CD22 single chain CAR), construct numbers D44, D47 (reference to D0044 and D0047 CAR constructs, respectively, zeroes between “D” and the numerical designation in the construct name were omitted for simplicity). DuoCARs and T cells and controls were incubated with triple positive Raji cells overnight, then supernatants were harvested and analyzed by ELISA for IFNg, TNFa and IL-2. N=3, +/−SD. One experiment representing three separate experiments in T cells from separate donors is shown.

FIG. 17 depicts a schematic representation of two CAR chains that can be combined for co-expression in the same cell or population of cells to generate DuoCARs by way of co-transfection or co-transduction. One CAR chain is comprised of CD22 scFv, linked in frame to CD8 hinge and transmembrane domain, 4-1BB costimulatory domain and CD3 zeta activation domain. Another CAR chain is comprised of a tandem CD20 CD19 scFv-based targeting domain, followed by CD8 hinge and transmembrane domain, CD28 costimulatory domain and CD3 zeta activation domain.

FIG. 18 depicts cell surface expression of DuoCARs and controls on primary human T cells transduced with DuoCAR expression vector preps generated by co-transfection of two transfer plasmids to produce LV or individually transduced single vector controls (top panel) as measured by flow cytometry. T cells were transduced to express the following CARs: construct numbers 2273, 2228 (-2019 tandem CAR), D1, D2, D3, CD22 CAR, and DuoCARs (construct numbers D1+2273, D2+2273, D3+2273). In scatter plots shown, the CAR 22 expression is shown on the Y axis, and CAR 19 expression, representing the tandem 2019 CAR chain, is shown on the X axis. Percentages of positive cells are denoted in each quadrant. Representative data for three experiments using T cells from three donors.

FIGS. 19A and 19B depict the anti-tumor cytolytic activity of DuoCAR cells or single chain CAR controls. The DuoCAR T cells were generated by co-transfection of two transfer plasmids to produce lentiviral vectors. T cells were transduced with the resulting DuoCAR vectors or with single chain CAR controls to express the following CARs: construct number 2273 (-the 2019 single chain tandem CAR); construct numbers D1, D2, D3 (−CD22 single chain CARs); and DuoCARs (construct numbers D1+2273, D2+2273, D3+2273, “D” in the designation omitted for brevity) generated by combination of two single CAR chains in the same CAR T product. The resulting CAR T cells were analyzed in a cytotoxic T cells assay at two different effector to target ratios (10:1, 5:1, as indicated) against native leukemia lines that are CD19+CD20+CD22+ (Raji, Reh) or CD19, CD20, CD22 triple-negative control line 293T (FIG. 19A). The native target lines Raji and Reh were lysed by single-chain CAR constructs by all DuoCAR groups construct numbers D1+2273, D2+2273, D3+2273, (“D” in the designation omitted for brevity), as well as by single chain CAR controls. By contract, DuoCARs and single CAR controls were not cytolytic vs the CD19, CD20, CD22-triple negative line 293T, demonstration target specificity of CAR constructs. Since DuoCARs target three target antigens simultaneously, and to further address the question of target-specificity, DuoCARs were tested against transgenic single-positive tumor lines generated on the background of K562 erythroleukemia cells, which are naturally devoid of CD19, CD20 or CD22 expression. The single-positive tumor cell lines used as CAR-T targets were: K19 (expresses CD19), K20 (expresses CD20), and K22 (expresses CD22), FIG. 19B. DuoCARs lysed single-positive cell lines in E:T dependent manner, indicating that all targeting domains of DuoCARs are functional, and specific to their cognate target molecules (FIG. 19B). Moreover, CAR single chain controls with mismatched antigen targeting domains (CAR 22, LTG 2200 vs K19 and K20, tandem CAR 2019, LTG 1479 vs K22) had no specific lytic activity (FIG. 19B).

FIG. 20 depicts the cytokine release activity of DuoCAR cells or single chain CAR controls in response to Raji13G11, a CD19+CD20+CD22+ clone. The DuoCAR T cells were generated by co-transfection of two transfer plasmids to produce lentiviral vectors. T cells were transduced with the resulting DuoCAR vectors of single chain CAR control vectors to express the following CARs: construct number 2273 (-2019 tandem CAR); construct numbers D1, D2, D3 (−CD22 CAR); and three DuoCARs (D1+2273, D2+2273, D3+2273, FIG. 20, “D” in the group labels omitted for brevity). The resulting CAR T cells were combined with the triple positive Raji tumor line at E:T ratio of 10 overnight and culture supernatants were analyzed for IFNg, TNFa and IL-2. All DuoCAR constructs elaborated high levels of the three cytokines in response to Raji cells. DuoCARs alone controls, comprised of CAR T cells incubated in the absence of Raji targets, produced no appreciable cytokines in response to Raji 13G11 cells, demonstrating that the cytokine response is target-specific (FIG. 20).

FIGS. 21A and 21B depict the construction of a DuoCARs targeting CD19, CD20 and CD22 simultaneously, as well as tandem and single CAR controls. Each DuoCAR is comprised of a tandem CD20 and CD19 dual targeting CAR, co-expressed with a first generation, or a second generation, single-targeting CD22 CAR. The two CAR constructs are co-expressed in a bicistronic format and are linked by ribosomal skip site 2A sequence, to assure stoichiometrically equal expression of the two CAR chains. Due to the nature of this bicistronic expression cassette, both CAR chains are co-expressed in each transduced T cell. FIG. 21A: The triple targeting anti-CD20 and anti-CD19 anti-CD22 DuoCAR D93 is comprised of 20-19 tandem ScFv, hinge and transmembrane domain, ICOS co-stimulatory domain and the CD3z activation domain, followed by the 2A sequence, and then the single targeting CD22 CAR comprised from CD22 scFv, hinge and transmembrane domain, and CD3z activation domain. DuoCAR D94 is constructed as D93, except for the substitution of the ICOS co-stimulatory domain for OX40 domain. DuoCAR construct D95 is constructed as D94, except for the addition of the ICOS co-stimulatory domain to the CD22 CAR chain. DuoCAR construct D96 is comprised as construct D95, except for the substitution of OX40 costimulatory domain in the D95 construct for the CD27 costimulatory domain. All constructs contain CD8-derived hinge and transmembrane domains. Tandem Construct 1497, and single CAR constructs D89, D92, 1538, 1497 represent functional controls. FIG. 21B schematically depicts DuoCAR T cell, in which one tandem CAR chain and one single CAR chain are co-expressed in the same T cell, a tandem CAR, a Single CAR of the second generation (with co-stimulatory domain), and a single CAR of the first generation (without costimulatory domain).

FIGS. 22A and 22B: Surface expression of Duo-CAR T constructs D93, D94, D95, D96 and a tandem CAR 1497 (comparison) on human primary T cells. CAR T expression was determined by flow cytometry. T cells were activated with Miltenyi Biotec TransAct™ CD3 CD28 reagent in the presence of IL-2, and transduced with LV at MOI 80, as described in Materials and Methods. On culture day 8, viable transduced T cells (7-AAD negative) were assayed for CAR surface expression using one of three staining methods: CD19 Fc followed by anti-Fc-AF647, CD20 biotin reagent followed by streptavidin PE, or CD22-his reagent followed by anti-his-PE staining. (FIG. 22A) One representative transduction experiment, out of four experiments, is shown. Expression of CD20-targeting scFv in relation to CD19 targeting scFv is shown in the top panel, and the expression of CD22 targeting scFv in relation to the expression of the CD19-targeting scFv is shown in the bottom panel. The LV used in transduction is listed on the top of each column. Percentage of CAR T-positive populations is noted in each quadrant of the histogram. (FIG. 22B) Mean percentage DuoCAR expression ±SEM for four transduction experiments performed in T cells from different donors is shown. CAR-transduced T cells were defined as CAR19+CAR22+ cells, representing simultaneous detection of the two DuoCAR chains co-expressed in each cell.

FIGS. 23A-23D depict CAR T cytotoxicity in vitro. Luciferase-based cytotoxicity assays were performed using, Raji 13G11 CD19+CD20+CD22+, REH CD19+CD20lowCD22+, or CD19-CD22-cell lines (293T or K562), stably transduced with luciferase. Specific lysis of target cells by DuoCARs DI-D4, tandem CAR 1497, or single CARs D89 and D92 is shown for (FIG. 23A) Raji cells, (FIG. 23B) Reh cells, (FIG. 23C) K562 cells or (FIG. 23D) 293T cells. Negative control UTD-untransduced T cells was included. CAR T cells and target tumor cells were co-incubated overnight at the listed effector to target (E:T) ratios, x-axis. Error bars represent mean values from three technical replicates. One experiment representing three separate experiments in T cells from three donors, is shown. Bars represent mean+/−SD values from three independent experiments performed with CAR T cells from three separate donors.

FIG. 24 depicts CAR T cytokine release in response to leukemia cell lines. Cytokine IL-2, IFNγ and TNFα production by CAR-T, listed on the x-axis, upon overnight co-culture with the Raji leukemia line at an E:T ratio of 10:1, was measured using ELISA. Bars represent mean+SD of three replicate samples. Data are representative of three independent experiments performed with CAR T cells from three separate donors.

FIGS. 25A and 25B depict in vivo anti-tumor activity of DuoCARs. NSG mice bearing Raji tumors were treated with DuoCAR T cells D93, D94, D95 and D96, or tandem CAR 1497, or single CARs D89 or D92. CAR T cells were injected i.v. seven days after tumor inoculation, either at dose of 5 million CAR T cells per mouse (FIG. 25A) or at two million CAR T cells per mouse (FIG. 25B). Tumor burden was evaluated by bioluminescence on days indicated. N=6 mice per group, mean Radiance±SEM is shown.

FIGS. 26A-26H depict CAR T cytotoxicity in vitro against A431 tumor line clones transduced to over express one target antigen only, in order to confirm the specificity of the CD22 CD19-targeting CAR T cells (FIGS. 26A-D), and the parental A431 negative control line, or Raji leukemia line clones engineered to lack expression of either CD19, CD20, or CD22, representing antigen-escaped clones, and the parental Raji line for comparison (FIGS. 26E-H). All target lines stably expressed firefly luciferase. Bars indicate mean±SEM values from triplicate determination from one experiment, representing three independent experiments performed with CAR T cells from three separate donors.

FIG. 27 depicts in vivo anti-tumor activity of DuoCARs in a model of tumor antigen escape. NSG mice were inoculated with a mixture of Raji CD19neg, Raji CD20neg, Raji CD22neg, and the parental Raji clone in equal proportions. Raji tumors were treated with DuoCAR T cells D93, D94, D95 or D96, which are capable of targeting CD19, CD20, or CD22 antigens, or single CAR controls: CAR22 D92, CAR19 1538, or CAR20 1495. T cells were injected i.v. seven days after tumor inoculation, at dose of 5 million CAR T cells per mouse or Tumor burden was evaluated by bioluminescence on days indicated. N=6 mice per group, mean Radiance±SEM is shown.

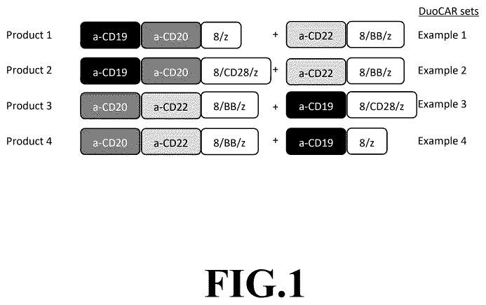

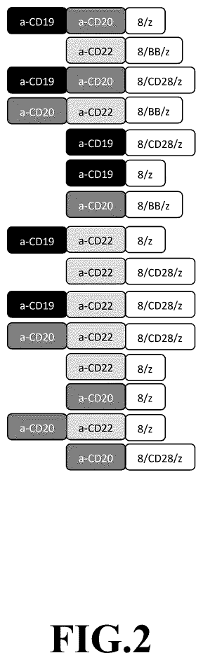

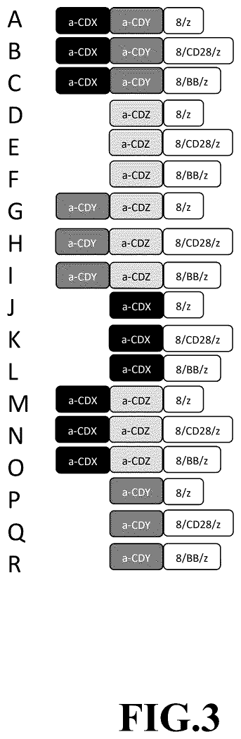

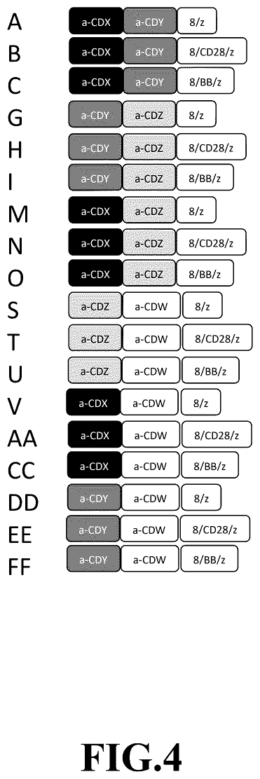

### DETAILED DESCRIPTION

### Definitions

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” Thus, “comprising an antigen” means “including an antigen” without excluding other elements. The phrase “and/or” means “and” or “or.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

The term “about” when referring to a measurable value such as an amount, a temporal duration, and the like, is meant to encompass variations of +/−20%, +/−10%, or more preferably +/−5%, or +/−1%, or still more preferably +/−0.1% from the specified value, as such variations are appropriate to perform the disclosed methods.

Unless otherwise noted, the technical terms herein are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: A Comprehensive Desk Reference, published by VCH Publishers, Inc., 1995; and other similar references.

The present invention relates to compositions and methods for treating diseases and/or conditions, as well as cancers including, but not limited to, hematologic malignancies and solid tumors. The present invention relates to a patient-specific, tumor-specific strategy of adoptive cell transfer of T cells transduced with two or more vectors to express one or more DuoCARs.

The present invention relates more particularly to lentiviral vectors expressing chimeric antigen receptors (DuoCARs), as well as host cells (e.g., lymphocytes, T cells) transduced with the lentiviral vectors expressing the CARs, nucleic acid molecules encoding the lentiviral vectors and chimeric antigen receptors, and methods of using same are also provided, for example, to treat a cancer in a subject.

Surprisingly and unexpectedly, it has now been discovered by the inventors that an immunotherapy composition comprising a patient-specific autologous anti-tumor lymphocyte cell population is much more effective as an anti-tumor immunotherapeutic if the autologous lymphocyte cell population is transduced with two or more lentiviral vectors encoding single or multiple chimeric antigen receptors (DuoCARs). The use of at least two or more lentiviral vectors expressing single or multiple CARS appears to promote in vivo expansion, persistence of patient-specific anti-tumor T-cells resulting in tumor stabilization, reduction, elimination, or remission of cancer, or prevention or amelioration of relapse of cancer, or any combination thereof, in a patient-specific manner.

Such active patient-specific anti-tumor T-cell populations as described herein can be infused directly back into the patient to promote in vivo expansion, persistence of patient-specific anti-tumor T-cells resulting in tumor stabilization, reduction, elimination, remission of cancer, or prevention or amelioration of relapse of cancer, or a combination thereof, in a patient-specific manner. This also includes effective expansion and rapid contraction of the therapeutic cell population.

Thus, in its broadest aspect, the novelty of this adoptive immunotherapy lies in the use of a combination of CAR-expression vectors. The differentiating feature is that contrary to the conventional use of a single vector expressing one or more chimeric antigen receptors, the Duo CAR approach confers both multiple antigen specificity and optimal signaling for anti-tumor T cell activity in vivo. Creating a system whereby three or more antigens are efficiently targeted is far superior to single or tandem approaches which allow for the tumor cancer cells to generate escape variants resulting in tumor metastasis and/or tumor relapse. The use of two or more vectors encoding single or multiple chimeric antigen receptors (DuoCARs) wherein the specific combination of least one binding domain(s) in each vector are non-identical coupled with the requirement that at least one signaling motif combination(s) are non-identical between each of the vectors, serves to ensure that genetically modified one or more lymphocyte populations transduced with such duo lentiviral vector-derived CARs generate a patient-specific autologous anti-tumor lymphocyte cell population capable of promoting in vivo expansion, persistence of patient-specific anti-tumor lymphocyte cells resulting in the stabilization, reduction, elimination, or remission of the tumor or cancer, and/or the prevention or amelioration of relapse of the tumor or cancer, or any combination thereof, in a patient-specific manner.

In one aspect, an immunotherapy composition is provided comprising one or more isolated nucleic acid molecules encoding at least two vectors (DuoCARs), each vector encoding a functional CAR, wherein at least one binding domain(s) in one of the vectors are non-identical, and whereby the combination of vectors results in the expression of two or more non-identical binding domains, wherein each vector encoded binding domain(s) are covalently linked to a transmembrane domain and one or more non-identical intracellular signaling motifs.

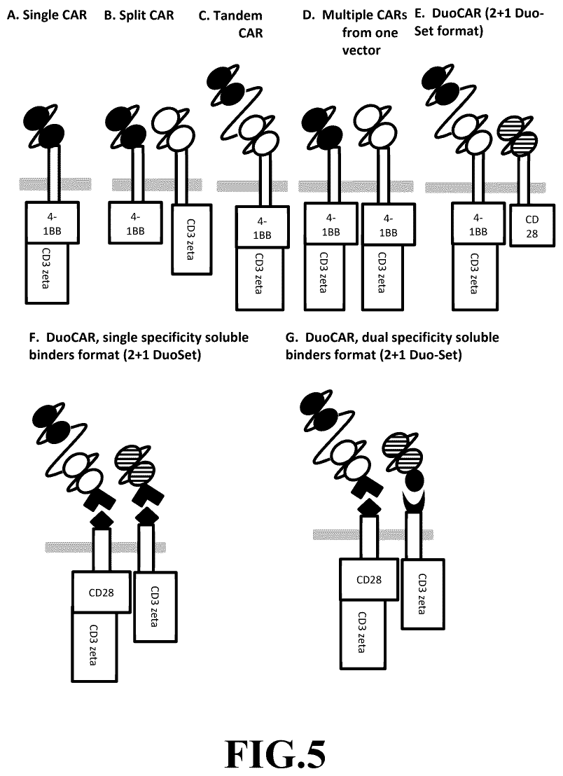

In another aspect, an immunotherapy composition is provided comprising one or more isolated nucleic acid molecules encoding at least two vectors (DuoCARs), each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs, with the proviso that said immunotherapy composition specifically excludes the single CARs, the Split CARs, the Tandem CARs, or the Multiple CARs depicted in FIG. 5 (A), (B), (C), or (D), respectively.

The immunotherapeutic efficacy and prevention or amelioration of relapse of the tumor or cancer achieved with the DuoCAR Lentiviral vector-modified T cells of the present invention is significantly greater and synergistically more than that achieved with the singular conventional CAR design. It is this unique combination of biological therapeutic benefits that correlates with the increased in vivo expansion, persistence of patient-specific anti-tumor lymphocyte cells resulting in the stabilization, reduction, elimination, or remission of the tumor or cancer compared to conventional CAR-based T-cell immunotherapy.

CAR expression vectors can be created that induce expression of a single binding domain (black, open or striped spheres, each with separate specificities, FIG. 5) connected to a linker and transmembrane domain (single open box). FIG. 5, infra, depicts a comparison of the conventional CARs versus the DuoCARs of the present invention. In FIG. 5, a thick gray line represents the plasma cell membrane. Intracellularly the lymphocyte signaling domains derived from 4-1BB (CD137), CD28, and the CD3-zeta chain can be combined as shown. In all examples and uses of the CD3 signaling domain in this document, included are modifications of the CD3 zeta chain by the alteration of either one, two, or three of the immunoreceptor tyrosine-based activation motifs (ITAM) by selective mutagenesis of the tyrosine residue therein, or other such mutations that render that ITAM motif to no longer be a target for phosphorylation. In Single CARs (FIG. 5A), a single binding domain is combined with a transmembrane and 2 signaling domains. In Split CARs (FIG. 5B), two different binders are expressed with single signaling domains that must be combined to render effective signaling. In Tandem CARs (FIG. 5C), two binding domains are linked to a single signaling domain. In Multiple CARs from one vector (FIG. 5D), two fully functional CARs are expressed from a single vector. The Duo-CARs of the present invention (e.g., FIG. 5E) encode at least two vectors, each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs. Essential features that differentiate the DuoCARs of the present invention is the use of two or more vectors, the multiplicity of binding domains, and the fully functional signaling characteristics (with regard to T cell expansion in vivo) of at least one of the two expressed cell surface proteins.

In another aspect, the DuoCARs are used to enhance the immune response to tumor mediated by the therapeutic T cell population. The immune response is enhanced in at least three ways.

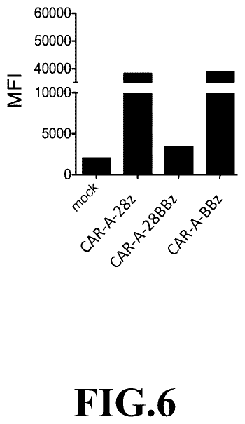

First, by providing the T cells an additional signal to expand and survive in the body, the DuoCARs of the present invention allow for the persistence of the therapeutic T cell population by virtue of stimulating the T cell population upon encountering self-antigen (for example CD19), whose loss can be tolerated by the patient, and yet which serves to provide a stimulatory signal for the therapeutic cellular population that does not reside in the tumor tissue itself. It is well known/established that third generation DuoCARs (expressing three co-stimulatory domains intracellularly, linked to a single extracellular Ig-like binder) are not expressed as well on therapeutic T cells compared to those DuoCARs expressing two intracellular co-stimulatory domains. For example, in FIG. 6 infra, the expression level of CAR constructs on primary human T cells differs between second generation (two costimulatory domains) and third generation (three costimulatory domains) constructs. T cells were transduced to express the following CARs: no CAR (mock), a second generation CAR (CAR-A-28z), a third generation CAR (CAR-A-28BBz), and an alternate second generation CAR (CAR-A-BBz). The level of surface expression of the CAR was detected by flow cytometry and is reported as mean fluorescence intensity (MF), y-axis. The MFI of both second-generation CARs was much brighter, even though all construct expressed the very same CAR binding domain.

By providing a third T cell activating sequence on a separate vector CAR construct, the inventors are able to regain the advantage of expressing three co-stimulatory domains, without incurring the disadvantage of the decreased expression of the CAR at the T cell surface.

In a second aspect, the DuoCARs of the present invention may target cell-types other than the tumor that mediate immunosuppressive effects. For example, if CD19-expressing B cells are present in the tumor lesion and also inhibit an anti-tumor immunity, as by the production of IL-4 or other mediators, the second benefit to the use of the DuoCAR-expressing tumor-specific T cell population is that the immunosuppressive cell population is also removed.

For example, if immunosuppressive B cells are present within a solid tumor lesion, these could be eliminated by the use of a B cell-specific DuoCAR (such as CD19-specific DuoCARs). If immunosuppressive fibroblast-like cells are present, these could be removed by stromal-specific DuoCARs (for example by targeting fibroblast activating protein-alpha (FAP)). If malformed vasculature is responsible for the lack of an efficacious immune response a DuoCAR specific for these types of vascular or lymph vessel specific targets (such as anti-VEGFR) may also improve therapeutic outcome.

In a third aspect, the DuoCARs of the present invention target an immunosuppressive population that is distal to the tumor, i.e. present in another compartment in the body. For example, using a DuoCAR to target myeloid derived suppressor cells (MDSCs), that may be present either in the tumor lesion itself or in the regional lymph nodes or bone marrow. It is well established that tumor-draining lymph nodes can either be loci of immune activation or immune suppression. This depends upon the overall inflammatory tone of the lymph node as well as distal dendritic cell differentiation prior to migration to the lymph node. If a tumor-draining lymph node is populated with myeloid-derived suppressor cells (MDSC) or miss-differentiated antigen presenting cells such as dendritic cells, a DuoCAR that targets these cell types, although distal to the tumor itself, may also improve therapeutic outcome. Beyond the cancer-specific DuoCAR immunotherapeutic applications, a second application of DuoCARs would be the prevention or treatment of autoimmune and/or inflammatory diseases. The difference from oncologic-based applications is that T-regulatory cells (Treg), or induced T-regulatory cells (iTreg), or other cells cultured in conditions that promote Th-2-like immune responses, would be the cellular substrate. For oncologic application Th-1 like cells are the cellular substrate. In therapeutic applications as diverse as graft-versus-host disease (GvHD) following hematopoietic stem cell transplantation (HSCT), allergic airway, gut, or other mucosal inflammation, or skin allergies, the presence of CAR-modified lymphocytes that produce immune-inhibitory cytokines, such as transforming growth factor-beta (TFG-beta), would serve to exert a broad tolerogenic signal that ameliorates the autoimmune- or inflammation-driven disease. This approach includes neurological inflammatory conditions of the periphery or central nervous system (CNS) such as Alzheimer's disease, multiple sclerosis, traumatic brain injury, Parkinson's disease, and CTE (chronic traumatic encephalopathy due to repeated concussions or micro-concussions). This approach also includes progressive scarring diseases such as COPD (chronic obstructive pulmonary disease).

In the treatment of inflammatory diseases, lymphocytes specific for tissue antigens, distress markers on the surface of inflamed cells, or misfolded proteins (such as tau protein or beta-amyloid) would be created by generating DuoCAR expression vectors that are specific for these targets. Single antibody-based therapy for Alzheimer's is already in clinical development (i.e., Solanezumab by Eli Lilly and Company and Aducanumab by Biogen, Inc.). In Alzheimer's disease, antibody to monomeric or aggregated beta-amyloid could be used in a CAR format in lieu of binders to cell surface proteins. Binders to tau protein or tau-peptides bound by MHC molecules could also be used as binding motifs for CARS. Receptors that mediate the homing of lymphocytes to specific peripheral tissues can also be included in a CAR format, in order to render regional specificity to the CAR-expressing Treg population. Adhesion receptor domains known to drive lymphocyte infiltration into specific tissues and cytokine sequences or cytokine or chemokine receptors or binders could be used as part of the CAR domain. Adhesion molecules such as CD44 and integrin alpha-4 are known to target lymphocytes to the CNS, thus including domains from adhesion molecules know to mediate CNS migratory behavior of lymphocyte populations could also be used to target CAR-expressing lymphocytes to regions of disease. The same would hold true for the gut (i.e. binders to MAdCAm-1, expression of a CCR9, or anti-CCL25, etc.), lung (i.e. P-selectin or mesothelin), skin (i.e. binders to E-selectin), or other mucosal surfaces.

To use this approach, a patient with an inflammatory condition or whose disease could be treated by mitigation of inflammatory pathology, such as Alzheimer's disease, would be admitted to the clinic and peripheral blood harvested. Treg could be selected directly by immunomagnetic beads (Regulatory T cell isolation kit, Miltenyi Biotec), or induced by culture in the appropriate cytokine milieu. These Treg or iTreg would then be transduced with a DuoCAR vector and if required expanded in vitro (Treg expansion kit, Miltenyi Biotec). The DuoCAR binding domains would be derived from antibodies or receptors that mediate tissue specific homing and disease-associated binders, such as anti-beta amyloid. The engineered immune effector cells thus generated would be targeted to the appropriate site, and produce cytokines consistent with their Th2 or Treg differentiation pattern. It is also known that CAR-T cells can be engineered to secrete specific genetic payloads upon activation of the CAR receptor. In addition to the DuoCAR payload expressed from the vector, additional therapeutic proteins or peptides could be expressed or secreted by the engineered T cell populations such as: a) A-beta DPs (amyloid beta degrading proteases), b) matrix proteases (such as MMP-9 and MMP9 inhibitors in COPD), c) peptides or soluble antibody-like binders that interfere with plaque formation, and d) cytokines (such as TGF-beta, IL-4, IL-10).

MiRNAs could also be expressed within cells to modulate T cell function. Examples of miRNAs are miR-92a, miR-21, miR-155, miR-146a, miR-3162, miR-1202, miR-1246 and miR-4281, miR-142, miR-17-92. Also shRNAs to miRNAs could be developed. Examples are shRNAs targeted to miR-28, miR-150 and miR-107, which normally bind to PDI and increase its expression.

Beyond oncology-based and inflammatory and autoimmune disease-based applications, a third application of the Duo CAR technology is the generation of therapeutic lymphocyte populations specific for viral, bacterial, or fungal antigens. Thus, as for oncology applications described for B cell malignancies, the targeting of infectious disease would allow the DuoCAR products to mediate immunoprotective or immunotherapeutic activity against the infective agents or the diseased tissues where they reside based upon recognition of microbial antigens. Unlike T cell receptor (TCR)-based approaches, where the T cell receptor itself mediates the recognition of pathogen encoded peptides, the Duo CAR approach would utilize binding proteins expressed in a CAR vector format that would give antibody-like recognition (that is, not requiring antigen processing) to the transduced T cell population. The activation of the therapeutic T cell population would result in an immune activating locus able to eliminate the infected cells, and if the microbial antigen is not cell associated, to release soluble mediators like interferon-gamma that would enable an effective immune response to be mounted against the infectious agent.

For example, HIV is known to be highly variable, and yet specific clades or families can be categorized and antibody to clade-specific viral envelope protein (env, gp120) created. Using the DuoCAR approach, three or more clade-specific antibody-like binders are included in the CAR constructs resulting in broad anti-HIV immune activity. In addition to viral proteins, bacterial protein can be targeted. A current medical challenge is the treatment of antibiotic resistant bacterial strains that often arise in healthcare settings. These include VRE (vancomycin resistant enterococci), MRSA (methicillin-resistant Staphylococcus aureus), KPC (Klebsiella pneumoniae carbapenemase producing gram-negative bacteria, also CRKP), and others. Klebsiella cell surface antigens include the O antigen (9 variants) and the K antigen (appx. 80 variants). The O antigen spectrum could readily be covered with a small DuoCAR library, as could a number of the K antigens. For use, CAR constructs would be created that feature antibodies that bind to different K or O serotypes, and these CAR vectors used to transduce a Th1-like effector cell population, isolated and activated as for oncology applications. In fungal diseases, the work of L. Cooper et al. (Kumasesan, P. R., 2014, PNAS USA, 111:10660) demonstrated that a fungal binding protein normally expressed on human cells, dectin-1, can be reconfigured as a CAR, and used to control fungal growth in vitro. The human disease aspergillosis occurs in severely immunosuppressed individuals and is caused by the fungus A. fumigatus. Multiple groups have produced monoclonal antibodies specific for the antigenic components of the aspergillus cell surface, thus opening the door to adoptive immunotherapy with DuoCARs that target three or more aspergillus antigens on the fungal surface. Thus, in all of these infectious disease applications, the ability to create immunoglobulin-like binders to microbial antigens allows a plurality of antigens to be targeted by CAR-expressing effector lymphocyte populations.

What follows is a detailed description of the DuoCARs that may be used in the patient-specific autologous anti-tumor lymphocyte cell population(s) disclosed herein, including a description of their extracellular domain, the transmembrane domain and the intracellular domain, along with additional description of the DuoCARs, antibodies and antigen binding fragments thereof, conjugates, nucleotides, expression, vectors, and host cells, methods of treatment, compositions, and kits employing the disclosed DuoCARs. While the compositions and methods of the present invention have been illustrated with reference to the generation and utilization of DuoCARs, it is contemplated herein that the compositions and methods are specifically intended to include the generation and utilization of TrioCARs and QuatroCARs.

### A. Chimeric Antigen Receptors (as Present in DuoCARs)

The DuoCARs disclosed herein comprise at least two vectors, each vector encoding a functional CAR, whereby the combination of vectors results in the expression of two or more non-identical binding domains, wherein each vector encoded binding domain(s) are covalently linked to a transmembrane domain and one or more non-identical intracellular signaling motifs, at least one extracellular domain capable of binding to an antigen, at least one transmembrane domain, and at least one intracellular domain.

A CAR is an artificially constructed hybrid protein or polypeptide containing the antigen binding domains of an antibody (e.g., single chain variable fragment (scFv)) linked to T-cell signaling domains via a transmembrane domain. Characteristics of DuoCARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing DuoCARs the ability to recognize antigen independent of antigen processing, thus bypassing a major mechanism of tumor escape. Moreover, when expressed in T-cells, DuoCARs advantageously do not dimerize with endogenous T cell receptor (TCR) alpha and beta chains.

As disclosed herein, the intracellular T cell signaling domains of the DuoCARs can include, for example, a T cell receptor signaling domain, a T cell costimulatory signaling domain, or both. The T cell receptor signaling domain refers to a portion of the CAR comprising the intracellular domain of a T cell receptor, such as, for example, and not by way of limitation, the intracellular portion of the CD3 zeta protein. The costimulatory signaling domain refers to a portion of the CAR comprising the intracellular domain of a costimulatory molecule, which is a cell surface molecule other than an antigen receptor or their ligands that are required for an efficient response of lymphocytes to antigen. In some instances the activation domains can be attenuated by the mutation of specific sites of phosphorylation, i.e. the ITAM motifs in the CD3 zeta chain, thus carefully modulating the degree of signal transduction mediated by that domain.

### 1. Extracellular Domain

In one embodiment, the CAR used in the patient-specific autologous anti-tumor lymphocyte cell population(s) as disclosed herein, comprises a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen binding domain in the CAR include those associated with viral, bacterial and parasitic infections, autoimmune disease and cancer cells.

In one embodiment, the CAR can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens are well known in the art and include, for example, a glioma-associated antigen, carcinoembryonic antigen (CEA), beta-human chorionic gonadotropin, alphafetoprotein (AFP), lectin-reactive AFP, thyroglobulin, RAGE-1, MN-CA IX, human telomerase reverse transcriptase, RUI, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I receptor, IGF-II receptor, IGF-I receptor and mesothelin. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20, CD22, and CD37 are other candidates for target antigens in B-cell lymphoma. Some of these antigens (CEA, HER-2, CD19, CD20, CD22, idiotype) have been used as targets for passive immunotherapy with monoclonal antibodies with limited success.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG16, TA-90\Mac-2 binding protein\cyclophilin C-associated protein, TAAL6, TAG72, TLP, and TPS.

In a preferred embodiment, the antigen binding domain portion of the CAR targets an antigen that includes but is not limited to CD19, CD20, CD22, ROR1, Mesothelin, CD33, c-Met, PSMA, Glycolipid F77, EGFRVIII, GD-2, MY-ESO-1 TCR, MAGE A3 TCR, and the like. In yet another embodiment, a DuoCAR is provided herein comprising a Tag or anti-Tag binding domain.

Depending on the desired antigen to be targeted, the CAR can be engineered to include the appropriate antigen binding domain that is specific to the desired antigen target. For example, if CD19 is the desired antigen that is to be targeted, an antibody or the scFv subfragment thereof specific for CD19 can be used as the antigen bind domain incorporated into the CAR.

In one exemplary embodiment, the antigen binding domain portion of the CAR targets CD19. Preferably, the antigen binding domain in the CAR is anti-CD19 scFV, wherein the nucleic acid sequence of the anti-CD19 scFV comprises the sequence set forth in SEQ ID NO: 27. In one embodiment, the anti-CD19 scFV comprises the nucleic acid sequence that encodes the amino acid sequence of SEQ ID NO: 28. In another embodiment, the anti-CD19 scFV portion of the CAR comprises the amino acid sequence set forth in SEQ ID NO: 28. In a second exemplary embodiment, the antigen binding domain of the CAR targets CD20. Preferably, the antigen binding domains in the CAR is anti-CD20 scFv, wherein the nucleic acid sequence of the anti-CD20 scFv comprises the sequence set forth in SEQ ID NO: 1. In another embodiment, the anti-CD20 scFV portion of the CAR comprises the amino acid sequence set forth in SEQ ID NO: 2. In a third exemplary embodiment, the antigen binding domain of the CAR targets CD22. Preferably, the antigen binding domains in the CAR is anti-CD22 scFv, wherein the nucleic acid sequence of the anti-CD22 scFv comprises the sequence set forth in SEQ ID NO: 7. In another embodiment, the anti-CD22 scFV portion of the CAR comprises the amino acid sequcne set forth in SEQ ID NO: 8.

In one aspect of the present invention, there is provided a CAR capable of binding to a non-TSA or non-TAA including, for example and not by way of limitation, an antigen derived from Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g. type 1 and type 2 herpes simplex virus (HSV), varicella-zoster virus, cytomegalovirus (CMV), and herpes virus], Poxviridae (e.g. smallpox virus, vaccinia virus, and pox virus), or hepatitis C virus, or any combination thereof.

In another aspect of the present invention, there is provided a CAR capable of binding to an antigen derived from a bacterial strain of Staphylococci, Streptococcus, Escherichia coli, Pseudomonas, or Salmonella. Particularly, there is provided a CAR capable of binding to an antigen derived from an infectious bacterium, for example, Helicobacter pyloris, Legionella pneumophilia, a bacterial strain of Mycobacteria sps. (e.g. M. tuberculosis, M. avium, M. intracellulare, M. kansaii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani, or a combination thereof.

### 2. Transmembrane Domain

In the DuoCARs used in the patient-specific autologous anti-tumor lymphocyte cell population(s) as disclosed herein, the CAR comprises one or more transmembrane domains fused to the extracellular domain of the CAR.

In one embodiment, an isolated nucleic acid molecule is provided wherein the encoded linker domain is derived from the extracellular domain of CD8, and is linked to the transmembrane domain.

In one embodiment, an isolated nucleic acid molecule is provided wherein the encoded linker domain is derived from the extracellular domain of the transmembrane domain and is linked to the transmembrane domain.

In some instances, the transmembrane domain can be selected or by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19, Fc epsilon R, or any combination thereof. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet or a triple alanine motif provides a particularly suitable linker.

In one embodiment, the transmembrane domain in the CAR of the invention is the CD8 transmembrane domain. In one embodiment, the CD8 transmembrane domain comprises the nucleic acid sequence of SEQ ID NO: 11. In one embodiment, the CD8 transmembrane domain comprises the nucleic acid sequence that encodes the amino acid sequence of SEQ ID NO: 12. In another embodiment, the CD8 transmembrane domain comprises the amino acid sequence of SEQ ID NO: 12.

In some instances, the transmembrane domain of the CAR comprises the CD8.alpha.hinge domain. In one embodiment, the CD8 hinge domain comprises the nucleic acid sequence of SEQ ID NO: 13. In one embodiment, the CD8 hinge domain comprises the nucleic acid sequence that encodes the amino acid sequence of SEQ ID NO: 14. In another embodiment, the CD8 hinge domain comprises the amino acid sequence of SEQ ID NO: 14.

Without being intended to limit to any particular mechanism of action, it is believed that possible reasons for the enhanced therapeutic function associated with the exemplary DuoCARs used in the patient-specific autologous anti-tumor lymphocyte cell population(s) as disclosed herein of the invention include, for example, and not by way of limitation, a) improved lateral movement within the plasma membrane allowing for more efficient signal transduction, b) superior location within plasma membrane microdomains, such as lipid rafts, and greater ability to interact with transmembrane signaling cascades associated with T cell activation, c) superior location within the plasma membrane by preferential movement away from dampening or down-modulatory interactions, such as less proximity to or interaction with phosphatases such as CD45, and d) superior assembly into T cell receptor signaling complexes (i.e. the immune synapse), or any combination thereof.

In one embodiment of the patient-specific autologous anti-tumor lymphocyte cell population(s) as disclosed herein, non-limiting exemplary transmembrane domains for use in the DuoCARs disclosed herein include the TNFRSF16 and TNFRSF19 transmembrane domains may be used to derive the TNFRSF transmembrane domains and/or linker or spacer domains as disclosed in Applicant's co-pending Provisional Patent Application No. 62/239,509, entitled CHIMERIC ANTIGEN RECEPTORS AND METHODS OF USE, as filed on Oct. 9, 2015, and assigned Lentigen Technology, Inc. matter number LEN_015PRO, including, in particular, those other TNFRSF members listed within the tumor necrosis factor receptor superfamily as listed in Table I therein.

### 3. Spacer Domain

In the DuoCARs used in the patient-specific autologous anti-tumor lymphocyte cell population(s) as disclosed herein, a spacer domain can be arranged between the extracellular domain and the TNFRSF transmembrane domain, or between the intracellular domain and the TNFRSF transmembrane domain. The spacer domain means any oligopeptide or polypeptide that serves to link the TNFRSF transmembrane domain with the extracellular domain and/or the TNFRSF transmembrane domain with the intracellular domain. The spacer domain comprises up to 300 amino acids, preferably 10 to 100 amino acids, and most preferably 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

As the spacer domain, the entire or a part of amino acid numbers 137 to 206 (SEQ ID NO: 15) which includes the hinge region of CD8.alpha. (NCBI RefSeq: NP.sub.-001759.3), amino acid numbers 135 to 195 of CD8.beta. (GenBank: AAA35664.1), amino acid numbers 315 to 396 of CD4 (NCBI RefSeq: NP.sub.-000607.1), or amino acid numbers 137 to 152 of CD28 (NCBI RefSeq: NP.sub.-006130.1) can be used. Also, as the spacer domain, a part of a constant region of an antibody H chain or L chain (CHI region or CL region, for example, a peptide having an amino acid sequence shown in SEQ ID NO: 16) can be used. Further, the spacer domain may be an artificially synthesized sequence.

Further, in the CAR, a signal peptide sequence can be linked to the N-terminus. The signal peptide sequence exists at the N-terminus of many secretory proteins and membrane proteins, and has a length of 15 to 30 amino acids. Since many of the protein molecules mentioned above as the intracellular domain have signal peptide sequences, the signal peptides can be used as a signal peptide for the CAR. In one embodiment, the signal peptide comprises the nucleotide sequence of the leader (signal peptide) sequence shown in SEQ ID NO: 5. In one embodiment, the signal peptide comprises the amino acid sequence shown in SEQ ID NO: 6.

### 4. Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARS disclosed herein include those derived from TCR zeta (CD3 Zeta), FcR gamma, FcR beta, CD3 gamma, CD3 delta, CD3 epsilon, CD5, CD22, CD79a, CD79b, and CD66d. Specific, non-limiting examples, of the ITAM include peptides having sequences of amino acid numbers 51 to 164 of CD3.zeta. (NCBI RefSeq: NP.sub.-932170.1), amino acid numbers 45 to 86 of Fc.epsilon.RI.gamma. (NCBI RefSeq: NP.sub.-004097.1), amino acid numbers 201 to 244 of Fc.epsilon.RI.beta. (NCBI RefSeq: NP.sub.-000130.1), amino acid numbers 139 to 182 of CD3.gamma. (NCBI RefSeq: NP.sub.-000064.1), amino acid numbers 128 to 171 of CD3.delta. (NCBI RefSeq: NP.sub.-000723.1), amino acid numbers 153 to 207 of CD3.epsilon. (NCBI RefSeq: NP.sub.-000724.1), amino acid numbers 402 to 495 of CD5 (NCBI RefSeq: NP.sub.-055022.2), amino acid numbers 707 to 847 of 0022 (NCBI RefSeq: NP.sub.-001762.2), amino acid numbers 166 to 226 of CD79a (NCBI RefSeq: NP.sub.-001774.1), amino acid numbers 182 to 229 of CD79b (NCBI RefSeq: NP.sub.-000617.1), and amino acid numbers 177 to 252 of CD66d (NCBI RefSeq: NP.sub.-001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein. In one embodiment, the cytoplasmic signaling molecule in the CAR comprises a cytoplasmic signaling sequence derived from CD3 zeta. In another embodiment one, two, or three of the ITAM motifs in CD3 zeta are attenuated by mutation or substitution of the tyrosine residue by another amino acid.

In a preferred embodiment, the intracellular domain of the CAR can be designed to comprise the CD3-zeta signaling domain by itself or combined with any other desired cytoplasmic domain(s) useful in the context of the CAR. For example, the intracellular domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such costimulatory molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Specific, non-limiting examples, of such costimulatory molecules include peptides having sequences of amino acid numbers 236 to 351 of CD2 (NCBI RefSeq: NP.sub.-001758.2), amino acid numbers 421 to 458 of CD4 (NCBI RefSeq: NP.sub.-000607.1), amino acid numbers 402 to 495 of CD5 (NCBI RefSeq: NP.sub.-055022.2), amino acid numbers 207 to 235 of CD8.alpha. (NCBI RefSeq: NP.sub.-001759.3), amino acid numbers 196 to 210 of CD83 (GenBank: AAA35664.1), amino acid numbers 181 to 220 of CD28 (NCBI RefSeq: NP.sub.-006130.1), amino acid numbers 214 to 255 of CD137 (4-1BB, NCBI RefSeq: NP.sub.-001552.2), amino acid numbers 241 to 277 of CD134 (OX40, NCBI RefSeq: NP.sub.-003318.1), and amino acid numbers 166 to 199 of ICOS (NCBI RefSeq: NP.sub.-036224.1), and their variants having the same function as these peptides have. Thus, while the disclosure herein is exemplified primarily with 4-1BB as the co-stimulatory signaling element, other costimulatory elements are within the scope of the disclosure.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to comprise the signaling domain of CD3-zeta and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-zeta and the signaling domain of CD28 and 4-1BB.

In one embodiment, the intracellular domain in the CAR is designed to comprise the signaling domain of 4-1BB and the signaling domain of CD3-zeta, wherein the signaling domain of 4-1BB comprises the nucleic acid sequence set forth in SEQ ID NO: 17 and the signaling domain of CD3-zeta comprises the nucleic acid sequence set forth in SEQ ID NO: 19.

In one embodiment, the intracellular domain in the CAR is designed to comprise the signaling domain of 4-1BB and the signaling domain of CD3-zeta, wherein the signaling domain of 4-1BB comprises the nucleic acid sequence that encodes the amino acid sequence of SEQ ID NO: 18 and the signaling domain of CD3-zeta comprises the nucleic acid sequence that encodes the amino acid sequence of SEQ ID NO: 20.

In one embodiment, the intracellular domain in the CAR is designed to comprise the signaling domain of 4-1BB and the signaling domain of CD3-zeta, wherein the signaling domain of 4-1BB comprises the amino acid sequence set forth in SEQ ID NO: 18 and the signaling domain of CD3-zeta comprises the amino acid sequence set forth in SEQ ID NO: 20.

### 5. Additional Description of DuoCARs

Also expressly included within the scope of the invention are functional portions of the DuoCARs used in the patient-specific autologous anti-tumor lymphocyte cell population(s) as disclosed herein. The term “functional portion” when used in reference to a CAR refers to any part or fragment of one or more of the DuoCARs disclosed herein, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the DuoCARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively, or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the DuoCARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The DuoCARs (including functional portions and functional variants) can be of any length, i.e., can comprise any number of amino acids, provided that the DuoCARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The DuoCARs (including functional portions and functional variants of the invention) can comprise synthetic amino acids in place of one or more naturally-occurring amino acids. Such synthetic amino acids are known in the art, and include, for example, aminocyclohexane carboxylic acid, norleucine,-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine,-aminocyclopentane carboxylic acid, α-aminocyclohexane carboxylic acid, a-aminocycloheptane carboxylic acid, a-(2-amino-2-norbornane)-carboxylic acid, γ-diaminobutyric acid, β-diaminopropionic acid, homophenylalanine, and a-tert-butylglycine.

The DuoCARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The DuoCARs (including functional portions and functional variants thereof) can be obtained by methods known in the art. The DuoCARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Methods of generating chimeric antigen receptors, T cells including such receptors, and their use (e.g., for treatment of cancer) are known in the art and further described herein (see, e.g., Brentjens et al., 2010, Molecular Therapy, 18:4, 666-668; Morgan et al., 2010, Molecular Therapy, published online Feb. 23, 2010, pages 1-9; Till et al., 2008, Blood, 1 12:2261-2271; Park et al., Trends Biotechnol., 29:550-557, 2011; Grupp et al., N Engl J Med., 368:1509-1518, 2013; Han et al., J. Hematol Oncol., 6:47, 2013; Tumaini et al., Cytotherapy, 15, 1406-1417, 2013; Haso et al., (2013) Blood, 121, 1165-1174; PCT Pubs. WO2012/079000, WO2013/126726; and U.S. Pub. 2012/0213783, each of which is incorporated by reference herein in its entirety). For example, a nucleic acid molecule encoding a disclosed chimeric antigen binding receptor can be included in an expression vector (such as a lentiviral vector) used to transduce a host cell, such as a T cell, to make the disclosed CAR. In some embodiments, methods of using the chimeric antigen receptor include isolating T cells from a subject, transducing the T cells with an expression vector (such as a lentiviral vector) encoding the chimeric antigen receptor, and administering the CAR-expressing T cells to the subject for treatment, for example for treatment of a tumor in the subject.

### B. Antibodies and Antigen Binding Fragments

One embodiment further provides a CAR used in the patient-specific autologous anti-tumor lymphocyte cell population(s) disclosed herein, a T cell expressing a CAR, an antibody, or antigen binding domain or portion thereof, which specifically binds to one or more of the antigens disclosed herein. As used herein, a “T cell expressing a CAR,” or a “CAR T cell” means a T cell expressing a CAR, and has antigen specificity determined by, for example, the antibody-derived targeting domain of the CAR.

As used herein, and “antigen binding domain” can include an antibody and antigen binding fragments thereof. The term “antibody” is used herein in the broadest sense and encompasses various antibody structures, including but not limited to monoclonal antibodies, polyclonal antibodies, multi-specific antibodies (e.g., bispecific antibodies), and antigen binding fragments thereof, so long as they exhibit the desired antigen-binding activity. Non-limiting examples of antibodies include, for example, intact immunoglobulins and variants and fragments thereof known in the art that retain binding affinity for the antigen.

A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. In some examples, a monoclonal antibody is an antibody produced by a single clone of B lymphocytes or by a cell into which nucleic acid encoding the light and heavy variable regions of the antibody of a single antibody (or an antigen binding fragment thereof) have been transfected, or a progeny thereof. In some examples monoclonal antibodies are isolated from a subject. Monoclonal antibodies can have conservative amino acid substitutions which have substantially no effect on antigen binding or other immunoglobulin functions. Exemplary methods of production of monoclonal antibodies are known, for example, see Harlow & Lane, Antibodies, A Laboratory Manual, 2nd ed. Cold Spring Harbor Publications, New York (2013).

Typically, an immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (2) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6.sup.th ed., W.H. Freeman and Co., page 91 (2007).) In several embodiments, the heavy and the light chain variable regions combine to specifically bind the antigen. In additional embodiments, only the heavy chain variable region is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). References to “VH” or “VH” refer to the variable region of an antibody heavy chain, including that of an antigen binding fragment, such as Fv, scFv, dsFv or Fab. References to “VL” or “VL” refer to the variable domain of an antibody light chain, including that of an Fv, scFv, dsFv or Fab.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, MD, 1991; “Kabat” numbering scheme), Al-Lazikani et al., (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a VH CDR3 is the CDR3 from the variable domain of the heavy chain of the antibody in which it is found, whereas a VL CDRI is the CDRI from the variable domain of the light chain of the antibody in which it is found. Light chain CDRs are sometimes referred to as LCDR1, LCDR2, and LCDR3. Heavy chain CDRs are sometimes referred to as LCDR1, LCDR2, and LCDR3.

An “antigen binding fragment” is a portion of a full length antibody that retains the ability to specifically recognize the cognate antigen, as well as various combinations of such portions. Non-limiting examples of antigen binding fragments include Fv, Fab, Fab′, Fab′-SH, F (ab′) 2; diabodies; linear antibodies; single-chain antibody molecules (e.g. scFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

A single-chain antibody (scFv) is a genetically engineered molecule containing the VH and VL domains of one or more antibody (ies) linked by a suitable polypeptide linker as a genetically fused single chain molecule (see, for example, Bird et al., Science, 242:423 426, 1988; Huston et al., Proc. Natl. Acad. Sci., 85:5879 5883, 1988; Ahmad et al., Clin. Dev. Immunol., 2012, doi: 10.1155/2012/980250; Marbry, IDrugs, 13:543-549, 2010). The intramolecular orientation of the VH-domain and the VL-domain in a scFv, is typically not decisive for scFvs. Thus, scFvs with both possible arrangements (VH-domain-linker domain-VL-domain; VL-domain-linker domain-VH-domain) may be used.

In a dsFv the heavy and light chain variable chains have been mutated to introduce a disulfide bond to stabilize the association of the chains. Diabodies also are included, which are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444 6448, 1993; Poljak et al., Structure, 2:1121 1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3rd Ed., W.H. Freeman & Co., New York, 1997.

Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1st Ed. New York: Cold Spring Harbor Laboratory Press, 2004. Print.; Lonberg, Nat. Biotech., 23:1117-1125, 2005; Lonenberg, Curr. Opin. Immunol., 20:450-459, 2008).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Methods of testing antibodies for the ability to bind to any functional portion of the CAR are known in the art and include any antibody-antigen binding assay, such as, for example, radioimmunoassay (RIA), ELISA, Western blot, immunoprecipitation, and competitive inhibition assays (see, e.g., Janeway et al., infra, U.S. Patent Application Publication No. 2002/0197266 A1, and U.S. Pat. No. 7,338,929).

Also, a CAR, a T cell expressing a CAR, an antibody, or antigen binding portion thereof, can be to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

### C. Conjugates

The DuoCARs used in the patient-specific autologous anti-tumor lymphocyte cell population(s) disclosed herein, a T cell expressing a CAR, or monoclonal antibodies, or antigen binding fragments thereof, specific for one or more of the antigens disclosed herein, can be conjugated to an agent, such as an effector molecule or detectable marker, using any number of means known to those of skill in the art. Both covalent and noncovalent attachment means may be used. Conjugates include, but are not limited to, molecules in which there is a covalent linkage of an effector molecule or a detectable marker to an antibody or antigen binding fragment that specifically binds one or more of the antigens disclosed herein. One of skill in the art will appreciate that various effector molecules and detectable markers can be used, including (but not limited to) chemotherapeutic agents, anti-angiogenic agents, toxins, radioactive agents such as ¹²⁵I, ³²P, ¹⁴C, ³H and ³⁵S and other labels, target moieties and ligands, etc.

The choice of a particular effector molecule or detectable marker depends on the particular target molecule or cell, and the desired biological effect. Thus, for example, the effector molecule can be a cytotoxin that is used to bring about the death of a particular target cell (such as a tumor cell).

The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, IL. The linker can be any molecule used to join the antibody or antigen binding fragment to the effector molecule or detectable marker. The linker is capable of forming covalent bonds to both the antibody or antigen binding fragment and to the effector molecule or detectable marker. Suitable linkers are well known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

In some embodiments, the linker is cleavable under intracellular conditions, such that cleavage of the linker releases the effector molecule or detectable marker from the antibody or antigen binding fragment in the intracellular environment. In yet other embodiments, the linker is not cleavable and the effector molecule or detectable marker is released, for example, by antibody degradation. In some embodiments, the linker is cleavable by a cleaving agent that is present in the intracellular environment (for example, within a lysosome or endosome or caveolea). The linker can be, for example, a peptide linker that is cleaved by an intracellular peptidase or protease enzyme, including, but not limited to, a lysosomal or endosomal protease. In some embodiments, the peptide linker is at least two amino acids long or at least three amino acids long. However, the linker can be 4, 5, 6, 7, 8, 9, 10, 11, 12, 13, 14 or 15 amino acids long, such as 1-2, 1-3, 2-5, 3-10, 3-15, 1-5, 1-10, 1-15 amino acids long. Proteases can include cathepsins B and D and plasmin, all of which are known to hydrolyze dipeptide drug derivatives resulting in the release of active drug inside target cells (see, for example, Dubowchik and Walker, 1999, Pharm. Therapeutics 83:67-123). For example, a peptide linker that is cleavable by the thiol-dependent protease cathepsin-B, can be used (for example, a Phenylalanine-Leucine or a Glycine-Phenylalanine-Leucine-Glycine linker). Other examples of such linkers are described, for example, in U.S. Pat. No. 6,214,345, incorporated herein by reference. In a specific embodiment, the peptide linker cleavable by an intracellular protease is a Valine-Citruline linker or a Phenylalanine-Lysine linker (see, for example, U.S. Pat. No. 6,214,345, which describes the synthesis of doxorubicin with the Valine-Citruline linker).

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) can be used. (See, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, for example, a thioether attached to the therapeutic agent via an acylhydrazone bond (see, for example, U.S. Pat. No. 5,622,929).

In other embodiments, the linker is cleavable under reducing conditions (for example, a disulfide linker). A variety of disulfide linkers are known in the art, including, for example, those that can be formed using SATA (N-succinimidyl-S-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene)-, SPDB and SMPT. (See, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3 (10): 1299-1304), or a 3′-N-amide analog (Lau et al., 1995, Bioorg-Med-Chem. 3 (10): 1305-12).

In yet other embodiments, the linker is not cleavable and the effector molecule or detectable marker is released by antibody degradation. (See U.S. Publication No. 2005/0238649 incorporated by reference herein in its entirety).

In several embodiments, the linker is resistant to cleavage in an extracellular environment. For example, no more than about 20%, no more than about 15%, no more than about 10%, no more than about 5%, no more than about 3%, or no more than about 1% of the linkers, in a sample of conjugate, are cleaved when the conjugate is present in an extracellular environment (for example, in plasma). Whether or not a linker is resistant to cleavage in an extracellular environment can be determined, for example, by incubating the conjugate containing the linker of interest with plasma for a predetermined time period (for example, 2, 4, 8, 16, or 24 hours) and then quantitating the amount of free effector molecule or detectable marker present in the plasma. A variety of exemplary linkers that can be used in conjugates are described in WO 2004-010957, U.S. Publication No. 2006/0074008, U.S. Publication No. 20050238649, and U.S. Publication No. 2006/0024317, each of which is incorporated by reference herein in its entirety.

In several embodiments, conjugates of a CAR, a T cell expressing a CAR, an antibody, or antigen binding portion thereof, and one or more small molecule toxins, such as a calicheamicin, maytansinoids, dolastatins, auristatins, a trichothecene, and CC1065, and the derivatives of these toxins that have toxin activity, are provided.

Maytansine compounds suitable for use as maytansinoid toxin moieties are well known in the art, and can be isolated from natural sources according to known methods, produced using genetic engineering techniques (see Yu et al (2002) PNAS 99:7968-7973), or maytansinol and maytansinol analogues prepared synthetically according to known methods. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, each of which is incorporated herein by reference. Conjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference.

Additional toxins can be employed with a CAR, a T cell expressing a CAR, an antibody, or antigen binding portion thereof. Exemplary toxins include Pseudomonas exotoxin (PE), ricin, abrin, diphtheria toxin and subunits thereof, ribotoxin, ribonuclease, saporin, and calicheamicin, as well as botulinum toxins A through F. These toxins are well known in the art and many are readily available from commercial sources (for example, Sigma Chemical Company, St. Louis, MO). Contemplated toxins also include variants of the toxins (see, for example, see, U.S. Pat. Nos. 5,079,163 and 4,689,401).

Saporin is a toxin derived from Saponaria officinalis that disrupts protein synthesis by inactivating the 60S portion of the ribosomal complex (Stirpe et al., Bio/Technology, 10:405-412, 1992). However, the toxin has no mechanism for specific entry into cells, and therefore requires conjugation to an antibody or antigen binding fragment that recognizes a cell-surface protein that is internalized in order to be efficiently taken up by cells.

Diphtheria toxin is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. Virol. 19:220, 1976), and has been used in human clinical trials. See, U.S. Pat. Nos. 5,792,458 and 5,208,021.

Ricin is the lectin RCA60 from Ricinus communis (Castor bean). For examples of ricin, see, U.S. Pat. Nos. 5,079,163 and 4,689,401. Ricinus communis agglutinin (RCA) occurs in two forms designated RCA60 and RCA120 according to their molecular weights of approximately 65 and 120 kD, respectively (Nicholson & Blaustein, J. Biochim. Biophys. Acta 266:543, 1972). The A chain is responsible for inactivating protein synthesis and killing cells. The B chain binds ricin to cell-surface galactose residues and facilitates transport of the A chain into the cytosol (Olsnes et al., Nature 249:627-631, 1974 and U.S. Pat. No. 3,060,165).

Ribonucleases have also been conjugated to targeting molecules for use as immunotoxins (see Suzuki et al., Nat. Biotech. 17:265-70, 1999). Exemplary ribotoxins such as α-sarcin and restrictocin are discussed in, for example Rathore et al., Gene 190:31-5, 1997; and Goyal and Batra, Biochem. 345 Pt 2:247-54, 2000. Calicheamicins were first isolated from Micromonospora echinospora and are members of the enediyne antitumor antibiotic family that cause double strand breaks in DNA that lead to apoptosis (see, for example Lee et al., J. Antibiot. 42:1070-87, 1989). The drug is the toxic moiety of an immunotoxin in clinical trials (see, for example, Gillespie et al., Ann. Oncol. 11:735-41, 2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B chain (abrin-b) binds to D-galactose residues (see, Funatsu et al., Agr. Biol. Chem. 52:1095, 1988; and Olsnes, Methods Enzymol. 50:330-335, 1978).

The CAR used in the patient-specific autologous anti-tumor lymphocyte cell population(s), a T cell expressing a CAR, monoclonal antibodies, antigen binding fragments thereof, specific for one or more of the antigens disclosed herein, can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP). A CAR, a T cell expressing a CAR, an antibody, or antigen binding portion thereof, can also be conjugated with enzymes that are useful for detection, such as horseradish peroxidase, β-galactosidase, luciferase, alkaline phosphatase, glucose oxidase and the like. When a CAR, a T cell expressing a CAR, an antibody, or antigen binding portion thereof, is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. A CAR, a T cell expressing a CAR, an antibody, or antigen binding portion thereof, may also be conjugated with biotin, and detected through indirect measurement of avidin or streptavidin binding. It should be noted that the avidin itself can be conjugated with an enzyme or a fluorescent label.

A CAR, a T cell expressing a CAR, an antibody, or antigen binding portion thereof, may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

A CAR, a T cell expressing a CAR, an antibody, or antigen binding portion thereof, can also be conjugated with a radiolabeled amino acid. The radiolabel may be used for both diagnostic and therapeutic purposes. For instance, the radiolabel may be used to detect one or more of the antigens disclosed herein and antigen expressing cells by x-ray, emission spectra, or other diagnostic techniques. Further, the radiolabel may be used therapeutically as a toxin for treatment of tumors in a subject, for example for treatment of a neuroblastoma. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### D. Nucleotides, Expression, Vectors, and Host Cells

Further provided by an embodiment of the invention is a nucleic acid comprising a nucleotide sequence encoding any of the DuoCARs, an antibody, or antigen binding portion thereof, described herein (including functional portions and functional variants thereof). The nucleic acids of the invention may comprise a nucleotide sequence encoding any of the leader sequences, antigen binding domains, transmembrane domains, and/or intracellular T cell signaling domains described herein.

In one embodiment, an isolated nucleic acid molecule encoding a chimeric antigen receptor (DuoCARs) is provided comprising, from N-terminus to C-terminus, at least one extracellular antigen binding domain, at least one transmembrane domain, and at least one intracellular signaling domain.

In one embodiment of the CAR used in the patient-specific autologous anti-tumor lymphocyte cell population(s), an isolated nucleic acid molecule encoding the CAR is provided wherein the encoded extracellular antigen binding domain comprises at least one single chain variable fragment of an antibody that binds to the antigen.

In another embodiment of the CAR used in the patient-specific autologous anti-tumor lymphocyte cell population(s), an isolated nucleic acid molecule encoding the CAR is provided wherein the encoded extracellular antigen binding domain comprises at least one heavy chain variable region of an antibody that binds to the antigen.

In yet another embodiment of the CAR used in the patient-specific autologous anti-tumor lymphocyte cell population(s), an isolated nucleic acid molecule encoding the CAR is provided wherein the encoded CAR extracellular antigen binding domain comprises at least one lipocalin-based antigen binding antigen (anticalins) that binds to the antigen.

In one embodiment of the CAR used in the patient-specific autologous anti-tumor lymphocyte cell population(s), an isolated nucleic acid molecule is provided wherein the encoded extracellular antigen binding domain is connected to the transmembrane domain by a linker domain.

In another embodiment of the DuoCARs used in the patient-specific autologous anti-tumor lymphocyte cell population(s), an isolated nucleic acid molecule encoding the CAR is provided wherein the encoded extracellular antigen binding domain is preceded by a sequence encoding a leader or signal peptide.

In yet another embodiment of the DuoCARs used in the patient-specific autologous anti-tumor lymphocyte cell population(s), an isolated nucleic acid molecule encoding the CAR is provided wherein the encoded extracellular antigen binding domain targets an antigen that includes, but is not limited to, CD19, CD20, CD22, ROR1, mesothelin, CD33/IL3Ra, CD38, CD123 (IL3RA), CD138, BCMA (CD269), GPC2, GPC3, FGFR4, c-Met, PSMA, Glycolipid F77, EGFRVIII, GD-2, NY-ESO-1 TCR, MAGE A3 TCR, or any combination thereof.

In certain embodiments of the DuoCARs used in the patient-specific autologous anti-tumor lymphocyte cell population(s), an isolated nucleic acid molecule encoding the CAR is provided wherein the encoded extracellular antigen binding domain comprises an anti-CD19 scFV antigen binding domain, an anti-CD20 scFV antigen binding domain, an anti-CD22 scFV antigen binding domain, an anti-RORI scFV antigen binding domain, an anti-TSLPR scFV antigen binding domain, an anti-mesothelin scFV antigen binding domain, an anti-CD33/IL3Ra scFV antigen binding domain, an anti-CD38 scFV antigen binding domain, an anti-CD123 (IL3RA) scFV antigen binding domain, an anti-CD138 scFV antigen binding domain, an anti-BCMA (CD269) scFV antigen binding domain, an anti-GPC2 scFV antigen binding domain, an anti-GPC3 scFV antigen binding domain, an anti-FGFR4 scFV antigen binding domain, an anti-c-Met scFV antigen binding domain, an anti-PMSA scFV antigen binding domain, an anti-glycolipid F77 scFV antigen binding domain, an anti-EGFRvIII scFV antigen binding domain, an anti-GD-2 scFV antigen binding domain, an anti-NY-ESo-1 TCR scFV antigen binding domain, an anti-MAGE A3 TCR scFV antigen binding domain, or an amino acid sequence with 85%, 90%, 95%, 96%, 97%, 98% or 99% identity thereof, or any combination thereof.

In one aspect of the DuoCARs used in the patient-specific autologous anti-tumor lymphocyte cell population(s), the DuoCARs provided herein further comprise a linker domain.

In one embodiment of the DuoCARs used in the patient-specific autologous anti-tumor lymphocyte cell population(s), an isolated nucleic acid molecule encoding the CAR is provided wherein the extracellular antigen binding domain, the intracellular signaling domain, or both are connected to the transmembrane domain by a linker domain.

In one embodiment of the DuoCARs used in the patient-specific autologous anti-tumor lymphocyte cell population(s), an isolated nucleic acid molecule encoding the CAR is provided wherein the encoded linker domain is derived from the extracellular domain of CD8, and is linked to the transmembrane domain.

In yet another embodiment of the DuoCARs used in the patient-specific autologous anti-tumor lymphocyte cell population(s), an isolated nucleic acid molecule encoding the CAR is provided wherein the nucleic acid sequence encoding the transmembrane domain comprises a nucleotide sequence with 85%, 90%, 95%, 96%, 97%, 98% or 99% identity thereof.

In one embodiment of the DuoCARs used in the patient-specific autologous anti-tumor lymphocyte cell population(s), an isolated nucleic acid molecule encoding the CAR is provided wherein the encoded transmembrane domain comprises an amino acid sequence comprising at least one but not more than 10 modifications, or a sequence with 85%, 90%, 95%, 96%, 97%, 98% or 99% identity thereof.

In another embodiment of the DuoCARs used in the patient-specific autologous anti-tumor lymphocyte cell population(s), an isolated nucleic acid molecule encoding the CAR is provided wherein the encoded CAR further comprises a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154, or a combination thereof.

In yet another embodiment of the DuoCARs used in the patient-specific autologous anti-tumor lymphocyte cell population(s), an isolated nucleic acid molecule encoding the CAR is provided wherein the encoded intracellular signaling domain further comprises a CD3 zeta intracellular domain.

In one embodiment of the CAR disclosed herein, an isolated nucleic acid molecule encoding the CAR is provided wherein the encoded intracellular signaling domain is arranged on a C-terminal side relative to the CD3 zeta intracellular domain.

In another embodiment of the DuoCARs used in the patient-specific autologous anti-tumor lymphocyte cell population(s), an isolated nucleic acid molecule encoding the CAR is provided wherein the encoded at least one intracellular signaling domain comprises a costimulatory domain, a primary signaling domain, or a combination thereof.

In further embodiments of the DuoCARs used in the patient-specific autologous anti-tumor lymphocyte cell population(s), an isolated nucleic acid molecule encoding the CAR is provided wherein the encoded at least one costimulatory domain comprises a functional signaling domain of OX40, CD70, CD27, CD28, CD5, ICAM-1, LFA-1 (CD11a/CD18), ICOS (CD278), DAP10, DAP12, and 4-1BB (CD137), or a combination thereof.

In one embodiment of the DuoCARs used in the patient-specific autologous anti-tumor lymphocyte cell population(s), an isolated nucleic acid molecule encoding the CAR is provided that further contains a leader sequence or signal peptide sequence.

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an embodiment of the invention, the nucleic acid may comprise a codon-modified nucleotide sequence that encodes the antigen binding domain of the inventive CAR. In another embodiment of the invention, the nucleic acid may comprise a codon-modified nucleotide sequence that encodes any of the DuoCARs described herein (including functional portions and functional variants thereof).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., supra. The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Integrated DNA Technologies (Coralville, IA, USA).

The nucleic acid can comprise any isolated or purified nucleotide sequence which encodes any of the DuoCARs or functional portions or functional variants thereof. Alternatively, the nucleotide sequence can comprise a nucleotide sequence which is degenerate to any of the sequences or a combination of degenerate sequences.

An embodiment also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive DuoCARs. It is generally appreciated that conditions can be rendered more stringent by the addition of increasing amounts of formamide.

Also provided is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA).

Bacteriophage vectors, such as λ\acute{ν}TIO, λ\ddot{∥}TI 1, λZapII (Stratagene), EMBL4, and λNMI 149, also can be used. Examples of plant expression vectors include pBIOl, pBI101.2, pBHOl. 3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector. A lentiviral vector is a vector derived from at least a portion of a lentivirus genome, including especially a self-inactivating lentiviral vector as provided in Milone et al., Mol. Ther. 17 (8): 1453-1464 (2009). Other examples of lentivirus vectors that may be used in the clinic, include, for example, and not by way of limitation, the LENTIVECTOR® gene delivery technology from Oxford BioMedica plc, the LENTIMAX™ vector system from Lentigen and the like. Nonclinical types of lentiviral vectors are also available and would be known to one skilled in the art.

A number of transfection techniques are generally known in the art (see, e.g., Graham et al., Virology, 52:456-467 (1973); Sambrook et al., supra; Davis et al., Basic Methods in Molecular Biology, Elsevier (1986); and Chu et al., Gene, 13:97 (1981).

Transfection methods include calcium phosphate co-precipitation (see, e.g., Graham et al., supra), direct micro injection into cultured cells (see, e.g., Capecchi, Cell, 22:479-488 (1980)), electroporation (see, e.g., Shigekawa et al., BioTechniques, 6:742-751 (1988)), liposome mediated gene transfer (see, e.g., Mannino et al., BioTechniques, 6:682-690 (1988)), lipid mediated transduction (see, e.g., Feigner et al., Proc. Natl. Acad. Sci. USA, 84:7413-7417 (1987)), and nucleic acid delivery using high velocity microprojectiles (see, e.g., Klein et al., Nature, 327:70-73 (1987)).

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

An embodiment further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

For purposes herein, the T cell can be any T cell, such as a cultured T cell, e.g., a primary T cell, or a T cell from a cultured T cell line, e.g., Jurkat, SupTI, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4+/CD8+ double positive T cells, CD4+ helper T cells, e.g., Thi and Th2 cells, CD8+ T cells (e.g., cytotoxic T cells), tumor infiltrating cells, memory T cells, naive T cells, and the like. The T cell may be a CD8+ T cell or a CD4+ T cell.

In an embodiment, the DuoCARs as described herein can be used in suitable non-T cells. Such cells are those with an immune-effector function, such as, for example, NK cells, and T-like cells generated from pluripotent stem cells.

Also provided by an embodiment is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

DuoCARs (including functional portions and variants thereof), nucleic acids, recombinant expression vectors, host cells (including populations thereof), and antibodies (including antigen binding portions thereof), can be isolated and/or purified. For example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

### E. Methods of Treatment

It is contemplated that the DuoCARs used in the patient-specific autologous anti-tumor lymphocyte cell population(s) can be used in methods of treating or preventing a disease in a mammal. In this regard, an embodiment provides a method of treating or preventing cancer in a mammal, comprising administering to the mammal the DuoCARs, the nucleic acids, the recombinant expression vectors, the host cells, the population of cells, the antibodies and/or the antigen binding portions thereof, and/or the pharmaceutical compositions in an amount effective to treat or prevent cancer in the mammal. Additional methods of use of the aforementioned DuoCARs have been disclosed supra.

An embodiment further comprises lymphodepleting the mammal prior to administering the DuoCARs disclosed herein. Examples of lymphodepletion include, but may not be limited to, nonmyeloablative lymphodepleting chemotherapy, myeloablative lymphodepleting chemotherapy, total body irradiation, etc.

For purposes of the methods, wherein host cells or populations of cells are administered, the cells can be cells that are allogeneic or autologous to the mammal. Preferably, the cells are autologous to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia (CLL), hairy cell leukemia, acute lymphocytic leukemia (ALL), acute myeloid leukemia (AML), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a mammal.

Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment provides a method of detecting the presence of cancer in a mammal, comprising: (a) contacting a sample comprising one or more cells from the mammal with the DuoCARs, the nucleic acids, the recombinant expression vectors, the host cells, the population of cells, the antibodies, and/or the antigen binding portions thereof, or the pharmaceutical compositions, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the method of detecting the presence of a proliferative disorder, e.g., cancer, in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

The contacting can take place in vitro or in vivo with respect to the mammal. Preferably, the contacting is in vitro.

Also, detection of the complex can occur through any number of ways known in the art. For instance, the DuoCARs disclosed herein, polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles) as disclosed supra.

Methods of testing a CAR for the ability to recognize target cells and for antigen specificity are known in the art. For instance, Clay et al., J. Immunol, 163:507-513 (1999), teaches methods of measuring the release of cytokines (e.g., interferon-γ, granulocyte/monocyte colony stimulating factor (GM-CSF), tumor necrosis factor a (TNF-a) or interleukin 2 (IL-2)). In addition, CAR function can be evaluated by measurement of cellular cytotoxicity, as described in Zhao et al., J. Immunol. 174:4415-4423 (2005).

Another embodiment provides for the use of the DuoCARs, nucleic acids, recombinant expression vectors, host cells, populations of cells, antibodies, or antigen binding portions thereof, and/or pharmaceutical compositions of the invention, for the treatment or prevention of a proliferative disorder, e.g., cancer, in a mammal. The cancer may be any of the cancers described herein.

Any method of administration can be used for the disclosed therapeutic agents, including local and systemic administration. For example, topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used; for example, a chemotherapeutic agent may be administered orally and an antibody or antigen binding fragment or conjugate or composition may be administered intravenously. Methods of administration include injection for which the CAR, CAR T Cell, conjugates, antibodies, antigen binding fragments, or compositions are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the antibody or antigen binding fragment to a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody or antigen binding fragment may be beneficial. In other examples, the conjugate is applied as an eye drop topically to the cornea, or intravitreally into the eye.

The disclosed therapeutic agents can be formulated in unit dosage form suitable for individual administration of precise dosages. In addition, the disclosed therapeutic agents may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Typical dosages of the antibodies or conjugates can range from about 0.01 to about 30 mg/kg, such as from about 0.1 to about 10 mg/kg.

In particular examples, the subject is administered a therapeutic composition that includes one or more of the conjugates, antibodies, compositions, DuoCARs, CAR T cells or additional agents, on a multiple daily dosing schedule, such as at least two consecutive days, 10 consecutive days, and so forth, for example for a period of weeks, months, or years. In one example, the subject is administered the conjugates, antibodies, compositions or additional agents for a period of at least 30 days, such as at least 2 months, at least 4 months, at least 6 months, at least 12 months, at least 24 months, or at least 36 months.

In some embodiments, the disclosed methods include providing surgery, radiation therapy, and/or chemotherapeutics to the subject in combination with a disclosed antibody, antigen binding fragment, conjugate, CAR or T cell expressing a CAR (for example, sequentially, substantially simultaneously, or simultaneously). Methods and therapeutic dosages of such agents and treatments are known to those skilled in the art, and can be determined by a skilled clinician. Preparation and dosing schedules for the additional agent may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, Md.

In some embodiments, the combination therapy can include administration of a therapeutically effective amount of an additional cancer inhibitor to a subject. Non-limiting examples of additional therapeutic agents that can be used with the combination therapy include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. These agents (which are administered at a therapeutically effective amount) and treatments can be used alone or in combination. For example, any suitable anti-cancer or anti-angiogenic agent can be administered in combination with the CARS, CAR-T cells, antibodies, antigen binding fragment, or conjugates disclosed herein. Methods and therapeutic dosages of such agents are known to those skilled in the art, and can be determined by a skilled clinician.

Additional chemotherapeutic agents for combination immunotherapy include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, vemurafinib, vandetanib, and tretinoin. Selection and therapeutic dosages of such agents are known to those skilled in the art, and can be determined by a skilled clinician.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used.

The combination therapy may provide synergy and prove synergistic, that is, the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the compounds are administered or delivered sequentially, for example by different injections in separate syringes. In general, during alternation, an effective dosage of each active ingredient is administered sequentially, i.e. serially, whereas in combination therapy, effective dosages of two or more active ingredients are administered together.

In one embodiment, an effective amount of an antibody or antigen binding fragment that specifically binds to one or more of the antigens disclosed herein or a conjugate thereof is administered to a subject having a tumor following anti-cancer treatment. After a sufficient amount of time has elapsed to allow for the administered antibody or antigen binding fragment or conjugate to form an immune complex with the antigen expressed on the respective cancer cell, the immune complex is detected. The presence (or absence) of the immune complex indicates the effectiveness of the treatment. For example, an increase in the immune complex compared to a control taken prior to the treatment indicates that the treatment is not effective, whereas a decrease in the immune complex compared to a control taken prior to the treatment indicates that the treatment is effective.

### F. Biopharmaceutical Compositions

Biopharmaceutical or biologics compositions (hereinafter, “compositions”) are provided herein for use in gene therapy, immunotherapy, adoptive immunotherapy, and/or cell therapy that include one or more of the disclosed DuoCARs, or T cells expressing a CAR, antibodies, antigen binding fragments, conjugates, DuoCARs, or T cells expressing a CAR that specifically bind to one or more antigens disclosed herein, in a carrier (such as a pharmaceutically acceptable carrier). The compositions can be prepared in unit dosage forms for administration to a subject. The amount and timing of administration are at the discretion of the treating clinician to achieve the desired outcome. The compositions can be formulated for systemic (such as intravenous) or local (such as intra-tumor) administration. In one example, a disclosed DuoCARs, or T cells expressing a CAR, antibody, antigen binding fragment, conjugate, is formulated for parenteral administration, such as intravenous administration. Compositions including a CAR, or T cell expressing a CAR, a conjugate, antibody or antigen binding fragment as disclosed herein are of use, for example, for the treatment and detection of a tumor, for example, and not by way of limitation, a neuroblastoma. In some examples, the compositions are useful for the treatment or detection of a carcinoma. The compositions including a CAR, or T cell expressing a CAR, a conjugate, antibody or antigen binding fragment as disclosed herein are also of use, for example, for the detection of pathological angiogenesis.

The compositions for administration can include a solution of the CAR, or T cell expressing a CAR, conjugate, antibody or antigen binding fragment dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents, adjuvant agents, and the like, for example, sodium acetate, sodium chloride, potassium chloride, calcium chloride, sodium lactate and the like. The concentration of a CAR, or T cell expressing a CAR, antibody or antigen binding fragment or conjugate in these formulations can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

A typical composition for intravenous administration includes about 0.01 to about 30 mg/kg of antibody or antigen binding fragment or conjugate per subject per day (or the corresponding dose of a CAR, or T cell expressing a CAR, conjugate including the antibody or antigen binding fragment). Actual methods for preparing administrable compositions will be known or apparent to those skilled in the art and are described in more detail in such publications as Remington's Pharmaceutical Science, 19th ed., Mack Publishing Company, Easton, PA (1995).

A CAR, or T cell expressing a CAR, antibodies, antigen binding fragments, or conjugates may be provided in lyophilized form and rehydrated with sterile water before administration, although they are also provided in sterile solutions of known concentration. The DuoCARs, or T cells expressing a CAR, antibody or antigen binding fragment or conjugate solution is then added to an infusion bag containing 0.9% sodium chloride, USP, and in some cases administered at a dosage of from 0.5 to 15 mg/kg of body weight. Considerable experience is available in the art in the administration of antibody or antigen binding fragment and conjugate drugs; for example, antibody drugs have been marketed in the U.S. since the approval of RITUXAN® in 1997. A CAR, or T cell expressing a CAR, antibodies, antigen binding fragments and conjugates thereof can be administered by slow infusion, rather than in an intravenous push or bolus. In one example, a higher loading dose is administered, with subsequent, maintenance doses being administered at a lower level. For example, an initial loading dose of 4 mg/kg antibody or antigen binding fragment (or the corresponding dose of a conjugate including the antibody or antigen binding fragment) may be infused over a period of some 90 minutes, followed by weekly maintenance doses for 4-8 weeks of 2 mg/kg infused over a 30 minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres, the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the DuoCARs, or T cells expressing a CAR, antibody or antigen binding fragment or conjugate compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44 (2): 58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### G. Kits

In one aspect, Kits employing the DuoCARs disclosed herein are also provided. For example, kits for treating a tumor in a subject, or making a CAR T cell that expresses one or more of the DuoCARs disclosed herein. The kits will typically include a disclosed antibody, antigen binding fragment, conjugate, nucleic acid molecule, CAR or T cell expressing a CAR as disclosed herein. More than one of the disclosed antibodies, antigen binding fragments, conjugates, nucleic acid molecules, DuoCARs or T cells expressing a CAR can be included in the kit.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, conjugates, nucleic acid molecules, DuoCARs or T cells expressing a CAR. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of a disclosed antibodies, antigen binding fragments, conjugates, nucleic acid molecules, DuoCARs or T cells expressing a CAR, for example, in a method of treating or preventing a tumor or of making a CAR T cell. The package insert typically includes instructions customarily included in commercial packages of therapeutic products that contain information about the indications, usage, dosage, administration, contraindications and/or warnings concerning the use of such therapeutic products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

### EXAMPLES

This invention is further illustrated by the examples of the DuoCARs depicted within the accompanying Figures infra and the disclosure at pages 17-27, inclusive supra, which examples are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein. Those skilled in the art will recognize, or be able to ascertain using no more than routine experimentation, many equivalents to the embodiments of the invention described herein. Such equivalents are encompassed by the following claims.

### DESCRIPTION OF EXAMPLES

Five examples are provided whereby the expression of three functional binding domains on the surface of a LV-transduced human T cell population, and combination of different co-stimulatory intracellular domains proves the feasibility of the DuoSet technology (Example 1), and the functional activity of this population against three different leukemia antigens proves its effectiveness (Example 2). Comparison of expression and function of DuoCARs generated co-transfection, aka transduction with single LV product encoding both DuoCAR chains (generated by co-transfection of the packaging line with two CAR encoding plasmids) are described in Example 3. In Example 4, DuoCARs transduced with LV generated by co-transfection method, and bicistronic DuoCARs encoded by a single construct, in which two DuoCAR chains are separated by a ribosomal skip site are compared for transduction efficiency and function.

Examples of the single specificity CARs on which this technology is based and which may be included as a DuoSet component in a DuoCAR include the single CD20 targeting vector LTG1495, nucleotide sequence SEQ ID NO: 3 and amino acid sequence SEQ ID NO: 4. A second example is the single specificity CAR LTG2200, specific for CD22, nucleotide sequence SEQ ID NO: 9 and amino acid sequence SEQ ID NO: 10. An important molecular aspect in creating DuoCARs is the inclusion of non-redundant compatible sequences, and the evaluation of those sequence in transduced T cells such that no untoward recombination or intracellular association occurs. This can occur both in the producer cell line of the vector, or in the target cell population. For this reason, variant CAR structures that are known to be compatible in the DuoCAR setting were included. These include the CD19-specific CAR LTG1494 described in nucleotide sequence SEQ ID: 29 and amino acid sequence SEQ ID: 30, respectively. This sequence includes the well-described linker that joins the heavy and light chains of the scFv referred to as the Whitlow linker (amino acid sequence GSTSGSGKPGSGEGSTKG (SEQ ID NO: 107), see Whitlow M., et al., 1993, Protein Eng. 6:989-995). In some cases the Whitlow linker was substituted for a (GGGGS)ₙ linker, for example in a CD19 CAR format, as in LTG1538, nucleotide sequence SEQ ID NO: 31 and amino acid sequence SEQ ID NO: 32, respectively. In another example CARs were created that have alternate transmembrane domains. The anti-CD19 CAR LTG1562, nucleotide sequence SEQ ID NO: 21 and amino acid sequence SEQ ID NO: 22, respectively, utilizes the CD4 (as opposed to CD8) transmembrane domain. Similarly the anti-CD19 CAR LTG1563 has an alternate transmembrane derived from TNFRSF19, nucleotide sequence SEQ ID NO: 49 and amino acid sequence SEQ ID NO:50, respectively. DuoCARs can also be targeted to solid tumors, for example those expressing the mesothelin tumor antigen. For example, scFV binders have been created for mesothelin, as disclosed in Applicant's co-pending Provisional Patent Application No. 62/444,201, entitled Compositions and Methods for Treating Cancer with Anti-Mesothelin Immunotherapy, as filed on Jan. 9, 2017, and assigned Lentigen Technology, Inc. matter number LEN_017, nucleotide sequence SEQ ID NO: 37 and amino acid sequence SEQ ID NO: 38, respectively, that can be incorporated into functional CARs, nucleotide sequence SEQ ID NO: 39 and amino acid sequence SEQ ID NO: 40, respectively, and that can thereby be incorporated into a DuoCAR therapy. In addition to scFv sequences, single chain antigen binders (as opposed to scFv) can be incorporated into a DuoCAR application. For example, the CD33-specific heavy chain only binder, as disclosed in Applicant's co-pending Provisional Patent Application No. 62/476,438, entitled Compositions and Methods For Treating Cancer With Anti-CD33 Immunotherapy, as filed on Mar. 24, 2017, and assigned Lentigen Technology, Inc. matter number LEN_018, nucleotide sequence SEQ ID NO: 41 and amino acid sequence SEQ ID NO: 42, respectively, can be incorporated into a functional CAR, LTG1906, nucleotide sequence SEQ ID NO: 43 and amino acid sequence SEQ ID NO: 44, respectively, that targets CD33-expressing malignancies. One example of a DuoCAR therapeutic application would be the treatment of leukemia that expresses the CD19, CD20, and TSLPR antigens. In this case, LTG1496 or LTG1497 (SEQ ID NOs: 35, 26, respectively) could be combined with a TSLPR-specific CAR (LTG1789), SEQ ID NO: 47 and amino acid sequence SEQ ID NO: 48, respectively, that had been created from TSLPR-specific scFV domains, nucleotide sequence SEQ ID NO: 45 and amino acid sequence SEQ ID NO: 46.

Another example of a DuoCAR therapeutic application would be the treatment of cancer that expresses the CD38 antigen. For instance, the CD38-specific binders, as disclosed in Applicant's co-pending Provisional Patent Application No. 62/773,940; entitled Compositions and Methods For Treating Cancer With Anti-CD38 Immunotherapy; as filed on Nov. 30, 2018; and assigned Lentigen Technology, Inc. matter number LEN_026; can be incorporated into one or more functional CARs that target CD38-expressing malignancies, as disclosed in Applicant's co-pending Provisional Patent Application No. 62/773,940, the entirety of which is incorporated by reference herein.

Another example of a DuoCAR therapeutic application would be the treatment of cancer that expresses the CD123 antigen. For instance, the CD123-specific binders, as disclosed in Applicant's co-pending U.S. patent application Ser. No. 16/578,063; entitled Compositions and Methods For Treating Cancer With Anti-CD123 Immunotherapy; as filed on Sep. 20, 2019; and assigned Lentigen Technology, Inc. matter number LEN_024; and claiming priority to Provisional Patent Application No. 62/734,106; as filed on Sep. 20, 2018; can be incorporated into one or more functional CARs that target CD123-expressing malignancies, as disclosed in Applicant's co-pending U.S. patent application Ser. No. 16/578,063, the entirety of which is incorporated by reference herein.

Another example of a DuoCAR therapeutic application would be the treatment of cancer that expresses the BCMA antigen. For instance, the BCMA-specific binders, as disclosed in Applicant's co-pending Provisional Patent Application No. 62/854,574; entitled Fully Human BCMA CAR T Cells for the Treatment of Multiple Myeloma and Other BCMA-Positive Malignancies; as filed on May 30, 2019; and assigned Lentigen Technology, Inc. matter number MBG_13; can be incorporated into one or more functional CARs that target BCMA-expressing malignancies, as disclosed in Applicant's co-pending Provisional Patent Application No. 62/854,574, the entirety of which is incorporated by reference herein.

Examples of tandem-CARs (containing 2 scFv domains, as described in nucleotide sequence SEQ ID: 23 and amino acid sequence SEQ ID: 24) on which this technology is based include the CD20_CD19 CAR LTG1497, nucleotide sequence SEQ ID NO: 25 and amino acid sequence SEQ ID NO: 26. In some cases reversing the order of the two binders may provide a better DuoCAR expression in target cells. Thus, LTG1497, where the CD19 scFv is more proximal, as shown in nucleotide sequence SEQ ID NO: 25 and amino acid sequence SEQ ID NO: 26; and LTG1496 where the CD19 scFV is more distal to the membrane, as shown in nucleotide sequence SEQ ID NO: 33 and amino acid sequence SEQ ID NO: 34, can both be used as one of the members of a DuoSet comprising a DuoCAR.

### Methods Utilized in Examples 1 and 2

### Cell Lines (PBMC and Targets)

All cell lines and reagents were purchased from American Tissue Culture Collection (ATCC, Manassas, VA), unless otherwise noted. The Burkitt lymphoma cell line Raji, the acute lymphocytic leukemia cell lines REH, as well as the chronic myelogenous leukemia cell line K562, were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). The human embryonic kidney cell line 293T was propagated in Dulbecco's modified Eagle medium supplemented with 10% heat-inactivated FBS.

Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones. The mouse-adapted Raji-luc line was generated by engrafting a Raji clone stably expressing firefly luciferase into NSG mice (NOD.Cg-Prkd^{scid} Il2rg^{tm1Wjl}/SzJ), The Jackson Laboratory Sacramento, CA), isolating the engrafted Raji-luc tumor cells from mouse spleens by either positive (CD19 microBeads, human, Miltenyi Biotec, Bergisch Gladbach, Germany) or negative selection (mouse cell depletion kit, Miltenyi Biotec), expanding in culture, and re-cloning to facilitate the selection of clones with high expression of firefly luciferase. Whole blood was collected from healthy volunteers at Oklahoma Blood Institute (OBI, Oklahoma City, OK) with donors' written consent. Processed buffy coats were purchased from OBI. The CD4-positive and CD8-positive human T cells were purified from buffy coats via positive selection using a 1:1 mixture of CD4- and CD8-MicroBeads (Miltenyi Biotec) according to manufacturer's protocol.

### Creation of Chimeric Antigen Receptor (CAR)-Expressing Vectors Comprising DuoCARs

CAR antigen-binding domains, scFv, sequences were derived from the mouse hybridoma FMC-63 for CD19 (FMC-63: AA 1-267, GenBank ID: HM852952.1) and Leu-16 for CD20 [1], entire sequence of VL and VH. The CD22 scFv binding was created from publicly available sequences. Tandem CAR19_20 or CAR20_19 were generated by linking scFv of each antibody in frame to CD8 hinge and transmembrane domains (AA 123-191, Ref sequence ID NP_001759.3), 4-1BB (CD137, AA 214-255, UniProt sequence ID Q07011) transactivation domain and CD3 zeta signaling domain (CD247, AA 52-163, Ref sequence ID: NP_000725.1.). The scFv regions of 19A and 20A were linked in sequence by a flexible interchain linker (GGGGS); (SEQ ID NO: 108), followed by CD8, 4-1BB and CD3 zeta domains. Leader sequence from human granulocyte macrophage colony stimulating factor receptor alpha subunit was included in all constructs, as described in [2]. CAR constructs sequences were codon optimized (DNA2.0, Newark, CA) and cloned into a third generation lentiviral plasmid backbone (Lentigen Technology Inc., Gaithersburg, MD) under the regulation of a human EF-1α promoter. Lentiviral vector (LV) containing supernatants were generated by transient transfection of HEK 293T cells, as previously described [3]. Harvested pelleted lentiviral supernatants were stored at −80° C.

### Primary T Cell Transduction:

Selected CD4+ and CD8+ human primary T cells from normal donors were cultivated in TexMACS medium (serum-free) supplemented with 40 IU/ml IL-2 at a density of 0.3 to 2×10⁶ cells/ml, activated with CD3/CD28 MACS® GMP TransAct reagent (Miltenyi Biotec) and transduced on day 3 with lentiviral vectors encoding CAR constructs in the presence of 10 μg/ml protamine sulfate (Sigma-Aldrich, St. Louis, MO) overnight, and media exchanged on day 4. On day 5, cultures were transferred to TexMACS medium supplemented with 200 IU/ml IL-2, and propagated until harvest on day 10-13.

Immune effector assays: To determine cell-mediated cytotoxicity (CTL assay), 5,000 target cells stably transduced with firefly luciferase were combined with CAR T cells at various effector to target ratios and incubated overnight. SteadyGlo reagent (Promega, Madison WI) was added to each well and the resulting luminescence was analyzed on an EnSpire plate reader (Perkin Elmer, Shelton, Connecticut) and recorded as counts per second (sample CPS). Target only wells (max CPS) and target only wells plus 1% Tween-20 (min CPS) were used to determine assay range. Percent specific lysis was calculated as: (1−(sample CPS−min CPS)/(max CPS−min CPS)).

Flow Cytometric analysis: All cell staining reagents for flow cytometry were from Miltenyi Biotec, unless otherwise noted. One million CAR T transduced cells were harvested from culture, washed two times in cold staining buffer (AutoMACS solution with 0.5% bovine serum albumin) and pelleted at 350 xg for 5 minutes at 4° C. CAR surface expression on transduced T cells was initially detected by staining with protein L-biotin conjugate (stock 1 mg/ml, 1:1000 dilution, GenScript, Piscataway, NJ) for 30 minutes at 4° C., followed by two washes and staining with streptavidin-PE conjugate for 30 minutes at 4° C. (stock: 1.0 ml, 1:200 dilution, Jackson ImmunoResearch Laboratories, West Grove, PA). Non-transduced cells and transduced cells stained with streptavidin-PE only, were used as negative controls. Anti-CD4 antibody was employed to determine CD4 to CD8 ratio of CAR T positive population, and was added during the second incubation step. Dead cells were excluded by 7AAD staining (BD Biosciences, San Jose, CA). Cells were washed twice and resuspended in 200 ul Staining Buffer before quantitative analysis by flow cytometry. Specific DuoSet CAR T staining was carried out on Human T cells activated with CD3-CD28 nanomatrix (TransAct, Miltenyi Biotec) transduced with DuoSet vectors in the presence of IL-2, and analyzed for expression of CD19-, CD20-, or CD22-scFv domains by flow cytometry using recombinant CD19, CD20, or CD22 for staining, as for antibodies.

Anti-CD19 scFv activity was detected with CD19-Fc (R&D Biosystems), used at 1 ug/sample, and stained with goat anti-human Fc-gamma-R-PE (Jackson ImmuoResearch Laboratories, Inc.) at 0.75 ug/smaple. Anti-CD20 scFv activity was detected with CD20-biotin (Miltenyi Biotech), 0.1 ug/sample, detected with streptavidinpAPC (Miltenyi Biotec) at 0.2 ug/sample. Anti-CD22 scFc activity was detected with CD22-His (Thermo Fisher) at 0.1 ug/sample, and detected with anti-His FITC (Miltenyi Biotec). Flow cytometric analysis was performed on a MACSQuantR 10 Analyzer (Miltenyi Biotec). Characterization of target tumor lines and luciferase-positive sub clones was performed using CD19-FITC, CD20 VioBlue, and CD22-APC antibodies. Dead cells were excluded from analysis by 7AAD staining (BD Biosciences, San Jose, CA).

### Example 1

### Expression of a DuoCAR (2+1 DuoSet) on Primary Human T Cells

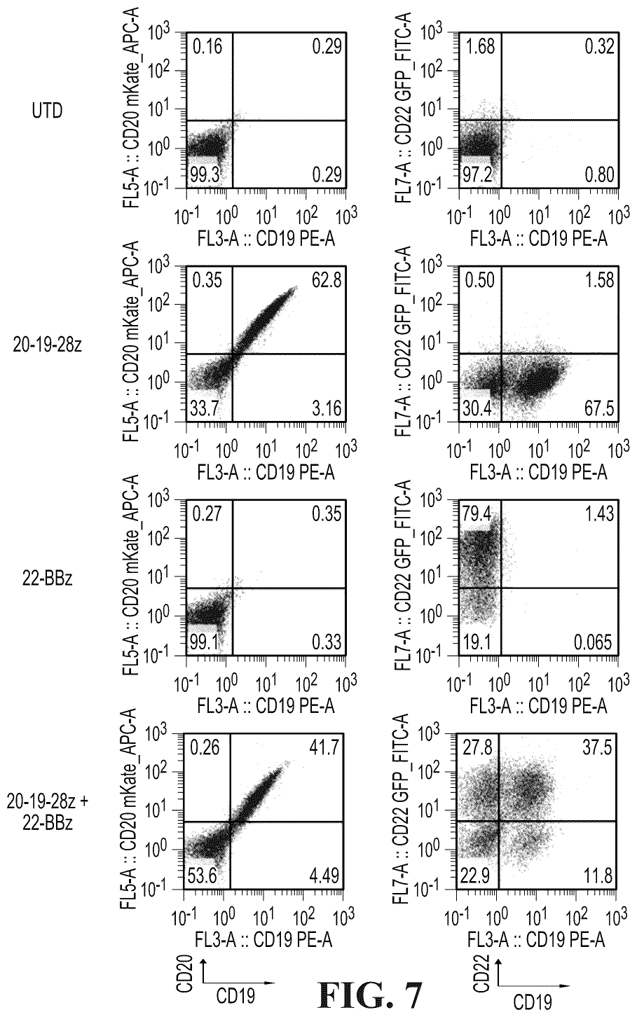

As a proof of principle, a DuoSet comprised of two CAR-T vectors was created. One member of the set expressed a tandem CD20_CD19 binding domain linked to CD8 transmembrane and CD28 and CD3-zeta signaling domains (LTG2228), SEQ ID NO: 51 and SEQ ID NO: 52. The second member of the DuoSet was a CAR construct with a single CD22 binder linked to CD8 transmembrane and 4-1BB and CD3-zeta signaling domains (LTG2200), SEQ ID NO: 9 and SED ID NO: 10. In FIG. 7, the paired columns show dual staining for CD20 and CD19 scFvs, left column, and CD22 and CD19 scFvs, right column. Row 1 shows

T cells that were not transduced (UTD) and thus show no binding. Row 2 shows T cells transduced with LV encoding a CD20_CD19 CAR vector with a CD8 transmembrane and intracellular CD28 and CD3-zeta signaling domains (20-19-28z). While dual staining is seen for CD20 and CD19 binding (left panel), only CD19 binding is seen in the right panel. Row 3 shows T cells transduced with a CD22 CAR vector with a CD8 transmembrane and intracellular 4-1BB and CD3-zeta signaling domains (22-BBz). No dual staining is seen with CD19 or CD20 (left panel) and only a single population of cells able to bind CD22 is seen (right panel). In Row 4 T cells are transduced with a DuoSet comprised of both vectors in Row 2 and Row 3. Only the DuoSet express all three CAR-encoded binding domains (42% of the cells express CD20_19 (left panel), and 38% expresses CD22 and CD19 bonding domains (right panel). As CD22 and CD19 scFv are on each of the two separate transmembrane proteins comprising the DuoSet, 38% represents the true DuoSet expressing population in this example.

### Example 2

### Anti-Leukemia Activity of a Human T Cell Preparation Expressing DuoCARs Generated by Co-Transduction Method

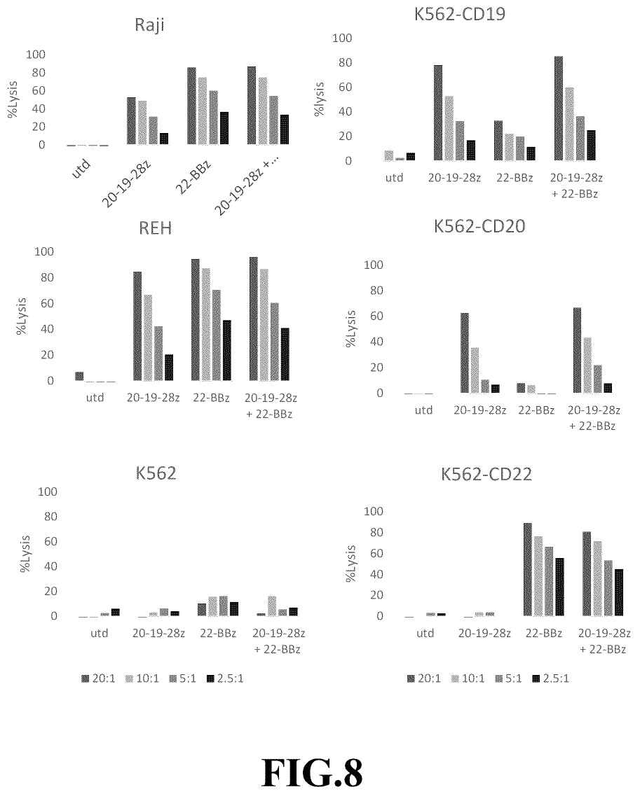

Anti-leukemia activity of a human T cell preparation expressing a DuoCAR that targets three leukemia antigens simultaneously (c.f., see FIG. 7 for DuoCAR expression characteristics). A DuoSet comprised of a CD20_19 tandem CAR and a CD22-specific single CAR (prepared as in Example 1) was used an effector T cell population in a cytotoxic T cell assay using leukemia cell line and model cell lines as targets. Human T cells transduced with single CAR components (20_19-28z or 22-BBz) or DuoCARs (20_19-28z+22-BBz), were used in cytotoxic T cells assay at four different effector to target ratios (20:1, 10:1, 5:1, 2.5:1, as indicated) (c.f., see FIG. 8). The leukemia cell lines used as CAR-T targets were: Raji (expresses all three target antigens), REH (expresses all three target antigens), K562 (control, no targets expressed), K562-CD19 (expresses CD19), K562-CD20 (expresses CD20), and K562-CD22 (expresses CD22). Only the DuoCAR-transduced cells (20-19-28z+22-BBz) exhibited high cytolytic activity against both leukemia cell lines (Raji and REH), and all three single-expressing K562 target cells lines (K562-CD19, K562-CD20, K562-CD22). This demonstrates that the DuoCAR technology can uniquely target three leukemia antigens simultaneously, in the same effector T cell population, and thus demonstrates superior anti-neoplastic activity by being able to target more than one or two target antigens at a time, thus decreasing the possibility of the malignancy generating escape mutants (cells clones that have lost or down-modulate one or two antigens and this escaped immune-ablation. The end result will be higher cure rates for patients, due to escape and outgrowth of antigen-loss variants, which in the end is a relapse.

### Example 3

### Anti-Leukemia Activity of a Human T Cell Preparation Expressing DuoCARs Generated by Co-Transfection Methods

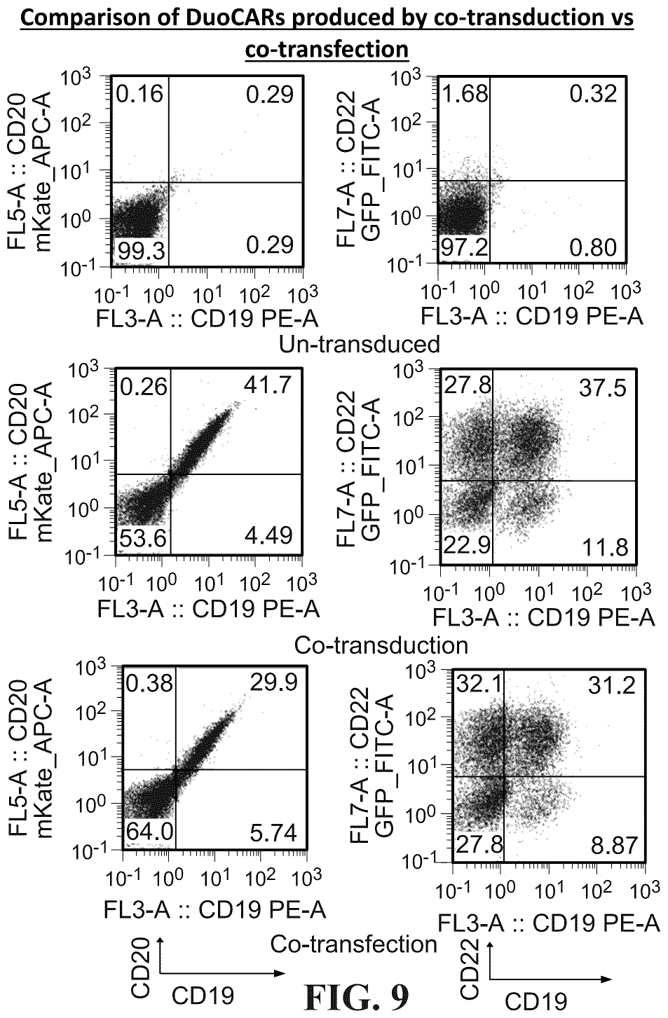

The DuoCAR technology described in this application generates a population of therapeutic lymphocytes, in this example human T cells, that express more than two antigen specificities from more than one transmembrane protein encoded by a gene vector. In this example, this is achieved by two different means. FIG. 9 contains three rows of data, labeled “un-transduced,” “co-transduction,” and “co-transfection”. FIG. 9 contains two columns of data, generated as in FIG. 7, wherein the first column is analyzed by flow cytometry for the expression of CD20- and CD19-specific specific binding, and the second column is analyzed by flow cytometry for the expression of CD22- and CD19-binding activity. In the first row of data, un-transduced human T cells are shown. No binding activity is seen for the CD19, CD20, or CD22 recombinant protein indicators of CAR-derived binding activity, demonstrating no DuoCAR expression. In the second row, “co-transduction” was used to generate DuoCARs. In this data set, two LV were used to simultaneously transduce activated T cells. As in FIG. 7, one CAR in the DuoSet comprising the DuoCAR was a tandem CD20 and CD19 binder linked to CD28 signaling and CD3-zeta signaling motifs; and the other CAR was a CD22 binder, linked to 4-1BB and CD3-zeta signaling motifs. The upper right quadrant in column one shows a very specific pattern of unitary staining for CD20 and CD19-scFv activity. This is due to both binders being on the same surface glycoprotein, and thus they are co-expressed with equal intensity, generating the very specific linear pattern seen. In the second column of the co-transduction data, a more traditional pattern is seen when the two glycoproteins are not expressed in a uniform pattern on each cell. Thus a pattern of 4 distinct populations is seen. In the lower left quadrant, cells expressing neither binder are seen. In the upper left, cells expressing only the CD22 CAR are seen. In the lower right quadrant cells expressing only the CD20_CD19 tandem CAR are seen. Finally, in the upper right quadrant cells expressing both members of the CAR DuoSet, comprising the DuoCAR, are seen.

In the bottom row, cell populations expressing the DuoCAR are generated in a different manner. Unlike the co-transduction method, where 2 LV preparations created independently are used at the time of the T cell transduction, “co-transfection” refers to a method wherein two backbone plasmids (encoding the two CARs comprising the DuoCAR) are simultaneously transfected into the 293T packaging cell line for LV production. The helper plasmids comprising this third generation LV system are identical in both methods. The advantage of the co-transfection method is that a single preparation of LV, containing vectors encoding both CARs is created. As can be seen from the data, using the co-transfection method nearly identical patterns of CD20-CD19 CAR and CD22 CAR expression are seen, as compared to the co-transduction method in the second row. The staining pattern for both glycoproteins induced by LV generated by co-transfection (CD22 for the CD22-CAR and CD19 co-staining for the CD20_19 CAR) in the upper right quadrant of the data in the second column, demonstrates that both methods efficiently generate DuoCARs.

### Referenced Literature

1) Wu, A. M., et al., Multimerization of a chimeric anti-CD20 single-chain Fv-Fc fusion protein is mediated through variable domain exchange. Protein engineering, 2001. 14 (12): p. 1025-1033.
2) Haso, W., et al., Anti-CD22-chimeric antigen receptors targeting B-cell precursor acute lymphoblastic leukemia. Blood, 2013. 121 (7): p. 1165-1174.
3) Kuroda, H., et al., Simplified lentivirus vector production in protein-free media using polyethylenimine-mediated transfection. Journal of virological methods, 2009. 157 (2): p. 113-121.

### Example 4

### Comparison of DuoCARs Generated by Co-Transfection Method and Bicistronic DuoCAR Constructs

### Methods Utilized in Example 4

Cell lines (PBMC and targets): All cell lines and reagents were purchased from American Tissue Culture Collection (ATCC, Manassas, VA), unless otherwise noted. The Burkitt's lymphoma cell line Raji, the acute lymphocytic leukemia cell lines REH, as well as the chronic myelogenous leukemia cell line K562, were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). The human embryonic kidney cell line 293T was propagated in Dulbecco's modified Eagle medium supplemented with 10% heat-inactivated FBS.

Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones. The mouse-adapted Raji-luc line was generated by engrafting a Raji clone stably expressing firefly luciferase into NSG mice (NOD.Cg-Prkd^{cscid} Il2rg^{tm1Wjl}/SzJ), The Jackson Laboratory Sacramento, CA), isolating the engrafted Raji-luc tumor cells from mouse spleens by either positive (CD19 microBeads, human, Miltenyi Biotec, Bergisch Gladbach, Germany) or negative selection (mouse cell depletion kit, Miltenyi Biotec), expanding in culture, and re-cloning to facilitate the selection of clones with high expression of firefly luciferase. Whole blood was collected from healthy volunteers at Oklahoma Blood Institute (OBI, Oklahoma City, OK) with donors' written consent. Processed buffy coats were purchased from OBI. The CD4-positive and CD8-positive human T cells were purified from buffy coats via positive selection using a 1:1 mixture of CD4- and CD8-MicroBeads (Miltenyi Biotec) according to manufacturer's protocol.

### Creation of Chimeric Antigen Receptor (CAR)-Expressing Vectors Comprising DuoCARs:

CAR antigen-binding domains, scFv, sequences were derived from the mouse hybridoma FMC-63 for CD19 (FMC-63: AA 1-267, GenBank ID: HM852952.1) and Leu-16 for CD20 [1], entire sequence of VL and VH. Several anti CD22 scFv binding sequences were used. Tandem CAR19_20 or CAR20_19 were generated by linking scFv of each antibody in frame to CD8 hinge and transmembrane domains (AA 123-191, Ref sequence ID NP_001759.3), 4-1BB (CD137, AA 214-255, UniProt sequence ID Q07011) transactivation domain and CD3 zeta signaling domain (CD247, AA 52-163, Ref sequence ID: NP_000725.1.). The scFv regions of 19A and 20A were linked in sequence by a flexible interchain linker (GGGGS)₅ (SEQ ID NO: 108), followed by CD8, 4-1BB and CD3 zeta domains. Leader sequence from human granulocyte macrophage colony stimulating factor receptor alpha subunit was included in all constructs, as described in [2]. In bicistronic CAR designs, two CAR chains were encoded within the same expression cassette, separated by ribosomal skip element 2A. CAR constructs sequences were codon optimized (DNA2.0, Newark, CA) and cloned into a third generation lentiviral plasmid backbone (Lentigen Technology Inc., Gaithersburg, MD) under the regulation of a human EF-1α of MSCV promoter. Lentiviral vector (LV) containing supernatants were generated by transient transfection of HEK 293T cells, as previously described [3]. For co-transfection experiments, equal amounts of two transfer plasmids encoding each of the DuoCAR chains were combined and applied, together with helper plasmids to the HEK 293T packaging cell line during transfection step, and resulting viral vector preparations were used for transduction of primary human T cells. Harvested pelleted lentiviral supernatants were stored at −80° C.

Primary T cell transduction: Selected CD4+ and CD8+ human primary T cells from normal donors were cultivated in TexMACS medium (serum-free) supplemented with 40 IU/ml IL-2 at a density of 0.3 to 2×10⁶ cells/ml, activated with CD3/CD28 MACS® GMP TransAct reagent (Miltenyi Biotec) and transduced on day 3 with lentiviral vectors encoding CAR constructs in the presence of 10 μg/ml protamine sulfate (Sigma-Aldrich, St. Louis, MO) overnight, and media exchanged on day 4. On day 5, cultures were transferred to TexMACS medium supplemented with 200 IU/ml IL-2, and propagated until harvest on day 10-13.

Immune effector assays: To determine cell-mediated cytotoxicity (CTL assay), 5,000 target cells stably transduced with firefly luciferase were combined with CAR T cells at various effector to target ratios and incubated overnight. SteadyGlo reagent (Promega, Madison WI) was added to each well and the resulting luminescence was analyzed on an EnSpire plate reader (Perkin Elmer, Shelton, Connecticut) and recorded as counts per second (sample CPS). Target only wells (max CPS) and target only wells plus 1% Tween-20 (min CPS) were used to determine assay range. Percent specific lysis was calculated as: (1−(sample CPS−min CPS)/(max CPS−min CPS)).

Flow Cytometric analysis: All cell staining reagents for flow cytometry were from Miltenyi Biotec, unless otherwise noted. One million CAR T transduced cells were harvested from culture, washed two times in cold staining buffer (AutoMACS solution with 0.5% bovine serum albumin) and pelleted at 350×g for 5 minutes at 4° C. CAR surface expression on transduced T cells was initially detected by staining with protein L-biotin conjugate (stock 1 mg/ml, 1:1000 dilution, GenScript, Piscataway, NJ) for 30 minutes at 4° C., followed by two washes and staining with streptavidin-PE conjugate for 30 minutes at 4° C. (stock: 1.0 ml, 1:200 dilution, Jackson ImmunoResearch Laboratories, West Grove, PA). Non-transduced cells and transduced cells stained with streptavidin-PE only, were used as negative controls. Anti-CD4 antibody was employed to determine CD4 to CD8 ratio of CAR T positive population, and was added during the second incubation step. Dead cells were excluded by 7AAD staining (BD Biosciences, San Jose, CA). Cells were washed twice and resuspended in 200 ul Staining Buffer before quantitative analysis by flow cytometry. Specific DuoSet CAR T staining was carried out on Human T cells activated with CD3-CD28 nanomatrix (TransAct, Miltenyi Biotec) transduced with DuoSet vectors in the presence of IL-2, and analyzed for expression of CD19-, CD20-, or CD22-scFv domains by flow cytometry using recombinant CD19, CD20, or CD22 for staining, as for antibodies.

Anti-CD19 scFv activity was detected with CD19-Fc (R&D Biosystems), used at 1 ug/sample, and stained with goat anti-human Fc-gamma-R-PE (Jackson ImmuoResearch Laboratories, Inc.) at 0.75 ug/sample. Anti-CD20 scFv activity was detected with CD20-biotin (Miltenyi Biotech), 0.1 ug/sample, detected with streptavidin APC (Miltenyi Biotec) at 0.2 ug/sample. Anti-CD22 scFv activity was detected with CD22-His (Thermo Fisher) at 0.1 ug/sample, and detected with anti-His FITC (Miltenyi Biotec). Flow cytometric analysis was performed on a MACSQuantR 10 Analyzer (Miltenyi Biotec). Characterization of target tumor lines and luciferase-positive sub clones was performed using CD19-FITC, CD20 VioBlue, and CD22-APC antibodies. Dead cells were excluded from analysis by 7AAD staining (BD Biosciences, San Jose, CA).

### Generating Bicistronic DuoCARs Using 2A Ribosomal Skip Sequence

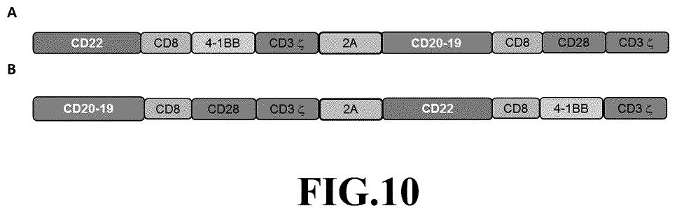

In addition to co-transduction and co-transfection approaches described in EXAMPLE 2 and EXAMPLE 3 supra, DuoCARs simultaneously targeting the three hematologic tumor antigens, CD19, CD20, CD22 and featuring different costimulatory domains, simultaneous expression of two CAR chains from a single mRNA transcript can be facilitated by use of self-cleavage element 2A. The 2A element mediates ribosomal skip during translation of the mRNA transcript to protein, thus enabling production of two discreet CAR protein chains at equimolar ratio. In this example, one CAR chain is comprised of CD22 scFv, linked in frame to CD8 hinge and transmembrane domain, 4-1BB costimulatory domain and CD3 zeta activation domain. The second CAR chain is comprised of a tandem CD20 CD19 scFv-based targeting domain, followed by CD8 hinge and transmembrane domain, CD28 costimulatory domain and CD3 zeta activation domain. The two designs differ in the order of CAR chains, such as in one design the CD22 CAR is first, followed by 2A element and the tandem 2019 CAR, and vice versa (FIG. 10).

First, a set of four bicistronic DuoCAR designs targeting CD19, CD20 and CD22 antigens simultaneously, under the control of EF1a promoter were constructed as described above (Set 1, Table 1 infra).

TABLE 1
list of Bicistronic DuoCAR Constructs and Single CAR Controls

| Bicistronic DuoCAR | | |
|---|---|---|
| Construct Number | Description | Set# |
| LTG2515 | EF1A-2019-28z-2A-m971-BBz | Set 1 |
| LTG2228 | EF1A-20-19-28z | Set 1 |
| LTG2520 | EF1A-2019-28z-2A-16P17-BBz | Set 1 |
| LTG2521 | EF1A-2019-28z-2A-16P8-BBz | Set 1 |
| LTG2200 | EF1A-m971 CD22 CAR control | Set 1 |
| LTG2209 | EF1A-16p17-BBz | Set 1 |
| LTG2218 | EF1A-16p8-BBz | Set 1 |
| D0043 | MSCV_20-19-28z-2A-m971-BBz | Set 2 |
| D0044 | MSCV_20-19-28z-2A-16p8-BBz | Set 2 |
| D0046 | MSCV_m971-BBz-2A-20-19-28z | Set 2 |
| D0047 | MSCV_16p8-BBz-2A-20-19-28z | Set 2 |

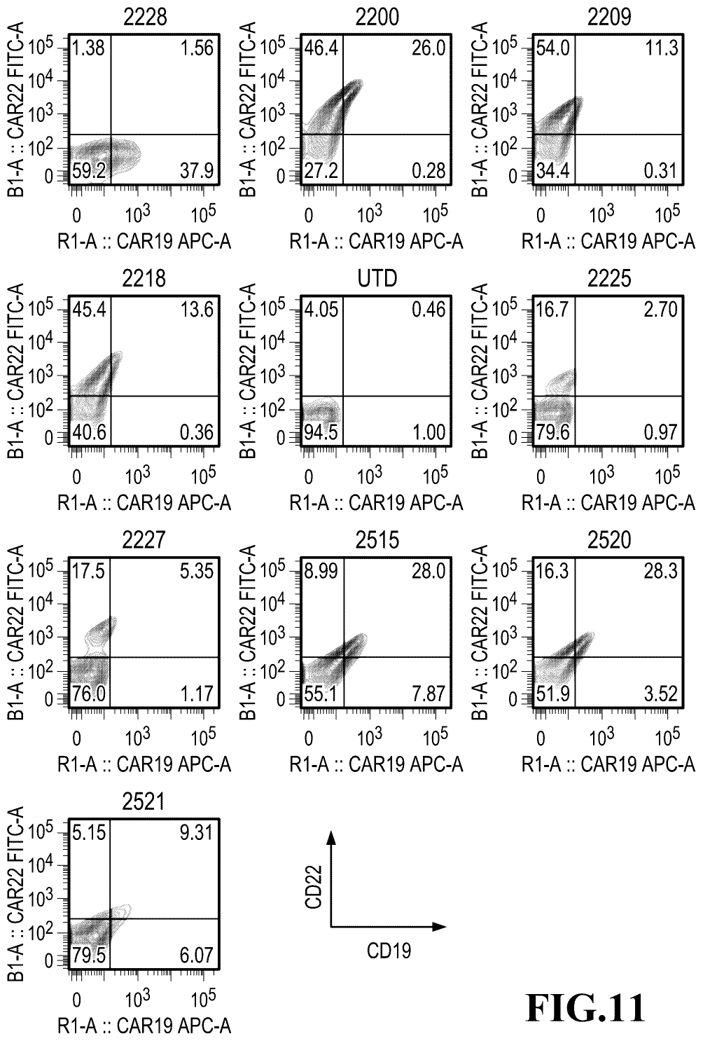

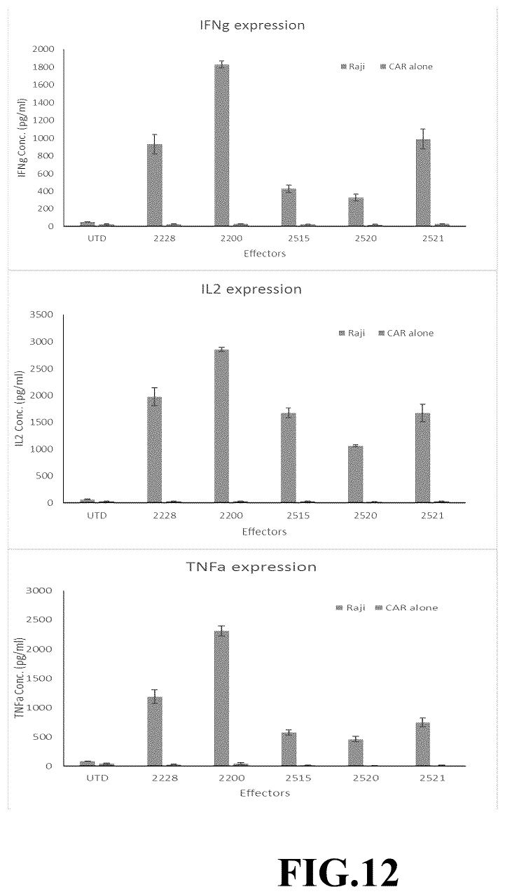

To facilitate optimal expression of CD22-targeting CAR moiety in DuoCAR format, the CD22-targeting CAR chain incorporated one of CD22-reactive scFv sequences 16P8, or 16P17. The CD22 scFv m971 was used as a comparator, and untransduced cells (UTD) served as a CAR-negative control). Co-expression of the CD20-CD19 targeting CAR chain and the CD22-targeting CAR chain was facilitated by 2A ribosomal skip sequence as described above. Individually encoded CAR chains were included as expression controls. Human primary T cells from a healthy donor were transduced with lentiviral vectors encoding each DuoCAR or single CAR control. Upon completion of T cell culture expansion, CAR expression was assessed by flow cytometry. The percentage of CAR20⁺CAR22⁺ double-positive cells in DuoCAR groups, representing co-expression of the tandem CD20-CD19 CAR chain and the CD22-CAR chain in the same cell, (LTG2515, LTG2520, LTG2521) was relatively low, and ranged from 28% (LTG2515, LTG2520) to 9% (LTG2521) (FIG. 11). By contrast, the expression of individual CAR controls was considerably greater, at ˜72% for CD22-targeting construct (LTG2200), and at ˜38% for the tandem CD20-CD19 targeting CAR (LTG 2228, FIG. 11). The functionality of DuoCARs was then tested in cytokine release assay. DuoCAR effector cells of controls were combined with Raji target cells at effector to target ratio (E:T) of 10 overnight. At the end of incubation period, cell culture supernatants were harvested and assayed for secreted T cell cytokines IFN gamma, TNF alpha and IL-2 (FIG. 12). Effectors incubated under similar conditions in the absence of tumor target cells were used as an additional control for spontaneous cytokine release. Co-incubation of Raji tumor cells with CAR effectors yielded strong upregulation of IFN gamma, IL-2 and TNFa for all constructs. Notably, none of the CARs produced cytokines spontaneously. However, the magnitude of cytokine secretion tended to be lower for all DuoCAR constructs as compared to positive controls CAR22 LTG2200, and tandem 2019 CAR LTG2228, likely due to relatively modest expression of the DuoCARs, as seen in FIG. 11.

Modest DuoCAR expression and cytokine response as compared to single CAR controls (FIG. 11, FIG. 12) suggested that the large payload size may be detrimentally impacting DuoCAR expression efficiency in the present configuration. In order to improve DuoCAR transduction efficiency, select DuoCAR sequences were codon re-optimized as needed, and expression cassettes were re-cloned into a new expression backbone, under the control of MSCV internal promoter for improved bicistronic expression (Set 2, Table 1).

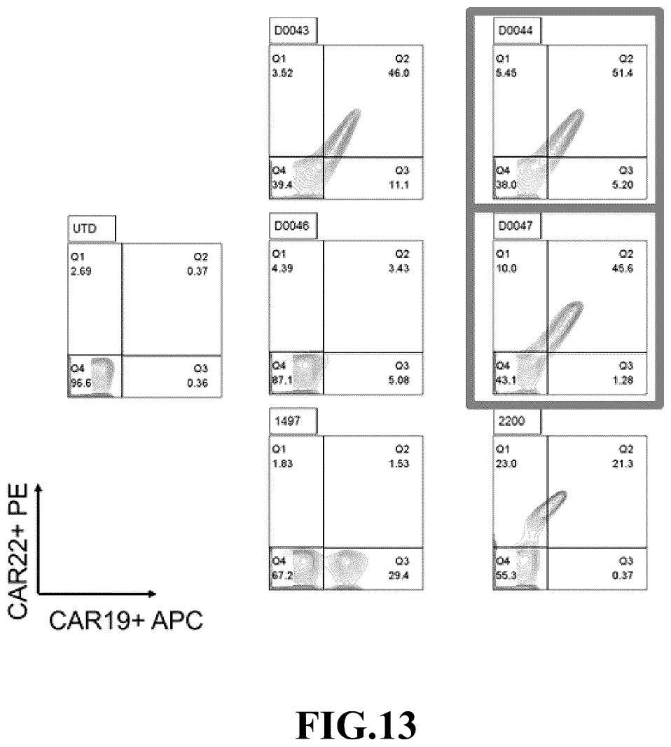

Lentiviral vectors were generated for each new DuoCAR construct, and CAR T cells were transduced and expanded as described in materials and methods. DuoCAR expression was determined by flow cytometry. The percentage of CD19+CD22+ T cells represents cells co-expressing the two chains of the DuoCAR (FIG. 13). Here, high transduction efficiency was achieved for DuoCAR Constructs D0044 (MSCV_20-19-28z-2A-16p8-BBz) and D0047 (MSCV_16p8-BBz-2A-20-19-28z), both containing the anti CD22 scFv 16P8 (FIGS. 13, 51% and 45%, respectively). Unexpectedly, DuoCAR D0043, containing the comparator m971 CD22 scFv was expressed well in the distal orientation (MSCV_20-19-28z-2A-m971-BBz, 46% positive), but showed no expression in the reverse orientation (D0046, MSCV_m971-BBz-2A-20-19-28z). Therefore the choice of scFv sequences included in DuoCAR design as well as sequence codon optimization and choice of expression backbone all are critical for optimal DuoCAR expression.

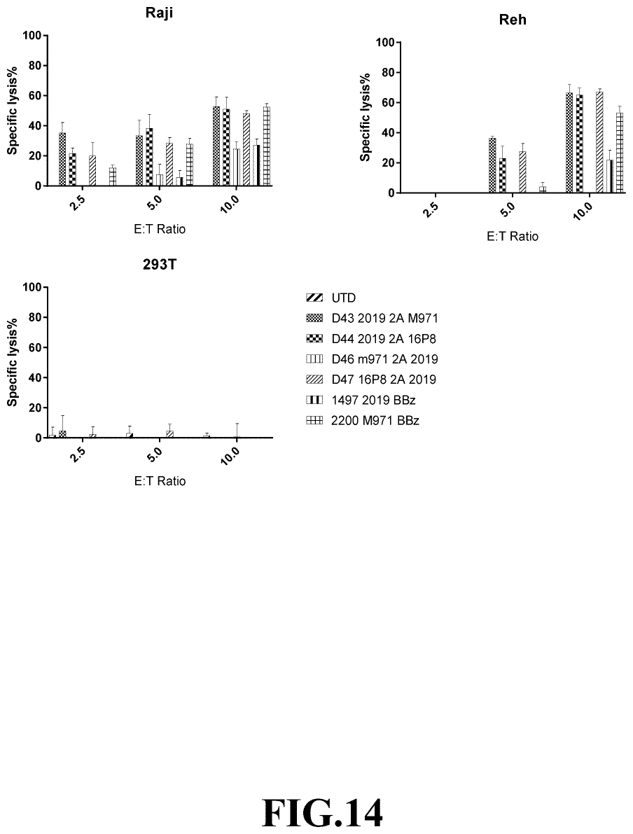

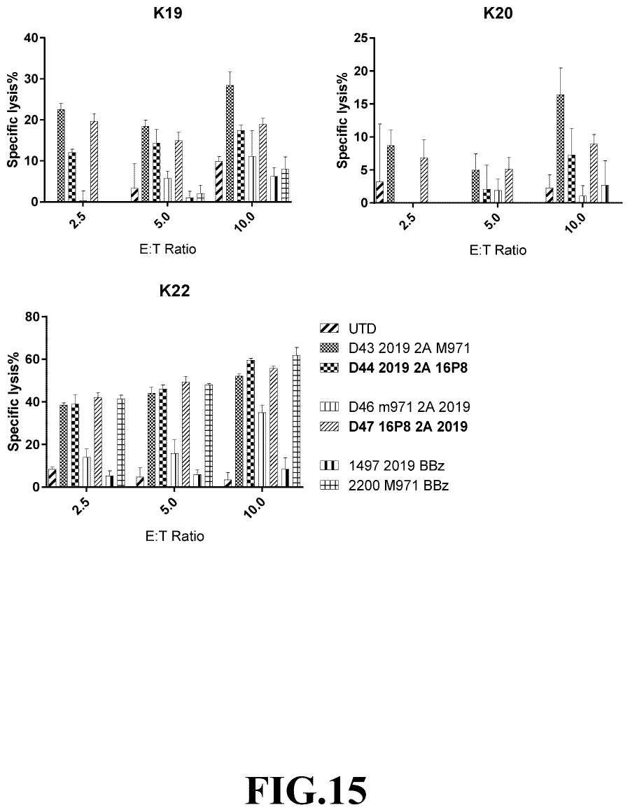

The cytotoxic function of DuoCAR set 2-transduced T cells was assayed in overnight killing assay vs a panel tumor lines with varying expression of tumor antigens CD19, CD20 and CD22. All lines were stably transduced to express firefly luciferase, and killing assays were performed as described in materials and methods. First, DuoCARs were combined with CD19+CD20+CD22+ with Non-Hodgkin's lymphoma Raji, or acute lymphoblastic leukemia Reh cells, or CD19-CD20-CD22-human embryonic kidney 293T cell line (FIG. 15). DuoCAR D0044 and D0047 bicistronically encoding CAR 20-19-28z and CD22 CAR 16p8-BBz CAR, and single CAR 22 control LTG2200, and tandem CAR control 20-19 LTG1497, as well as untransduced T cell control (UTD) were included (FIG. 14) Constructs D0043, D0044, D0046 and D0047 are noted in figure legend as D43, D44, D46 and D47, respectively, for brevity (FIG. 14). Effector and target cells were incubated at ratios of 2.5, 5 or 10 overnight in triplicate, then plates were harvested and developed with SteadyGlo reagent, and luciferase activity of the surviving tumor cells was determined by luminometry. Overall, CAR cytolytic function correlated with DuoCAR expression (FIG. 13). DuoCARs D0047 and D0044 potently lysed CD19, CD20 and CD22 triple-positive tumor lines Raji and Reh, as did the positive control DuoCAR D0043, whereas the sub-optimally expressed construct D0046 had relatively low lytic function (FIG. 14). No lysis of the CD19-CD20-CD22-triple negative line was caused by either CAR construct, underscoring the specificity of CAR-mediated lysis to cognate antigens.

To further delineate the specificity of DuoCAR constructs, transgenic K562 lines expressing either CD19, CD20 or CD22 antigens, termed K19, K20, K22, respectively were generated (FIG. 15). In co-incubation assays with single-positive tumor lines, DuoCARs D44 and D47, featuring CAR chains targeting CD19, CD20 and CD22, potently lysed each target line in effector to target ratio dependent manner, and were similar in their function to the comparator DuoCAR D0043 (construct designations in figure legends were shortened from D0043, D0044, D0046, D0047 to D43, D44, D46 and D47, respectively-FIG. 15). Control T cells expressing a tandem 2019 CAR (1497), lysed tumor lines K19 and K20, but had only negligible background lytic effect in K22 (less than 10% lysis at the highest E:T ratio of 10). The single CD22 control CAR potently lysed K22 tumor cells, but had no function in K20 cells, and only showed background lysis in K19 cells (10% lysis at the highest E:T of 10:1). Therefore, this experimental system enables testing of CAR reactivity to each tumor antigen with high accuracy. In summary, both DuoCARs D0044 and D0047 demonstrated that each of their tumor targeting domains is functional in this single antigen expressing test system (FIG. 15).

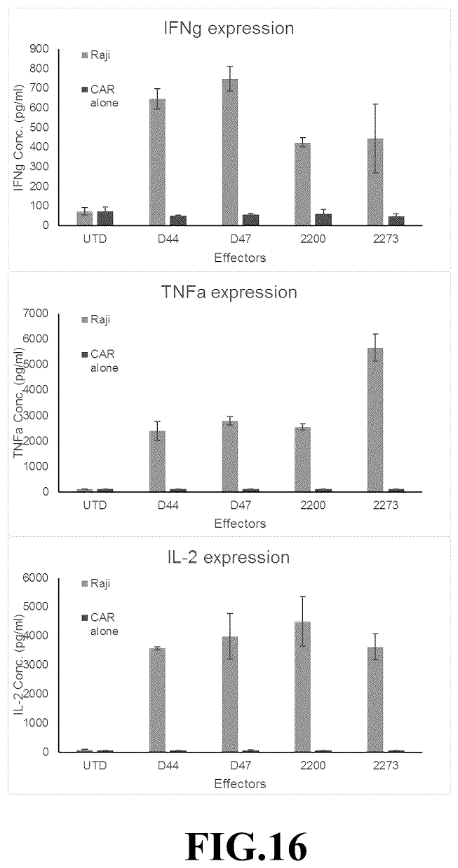

To characterize the cytokine release response of DuoCAR constructs, each of the DuoCAR T cell preparations D0044, D0047 (Figure legend: D44, D47, respectively) with the CD19+CD20+CD22+ were combined with Raji tumor cells at E:T ratio of 10 overnight, and analyzed culture supernatants by ELISA for T cell cytokines IFNg, TNFa and IL-2 (FIG. 16). Single CAR22 construct LTG2200 and Tandem 2019 CAR construct LTG2273 were included for comparison, and untransduced T cells (UTD) were used a s a negative control. In parallel, CAR T cells from each group were incubated under similar conditions but in the absence of tumor cells, to test for spontaneous cytokine release (FIG. 16). It was found that whereas none of the constructs yielded spontaneous release of cytokines, both DuoCARs D44 and D47 manifested strong induction of IL-2, IFNg and TNFa after co-incubation with Raji targets, underscoring the potency of these DuoCAR constructs. Notably, despite co-expressing two chains simultaneously in the same cell, no evidence of tonic signaling was detected, as attested by complete absence of spontaneous cytokine release (FIG. 16).

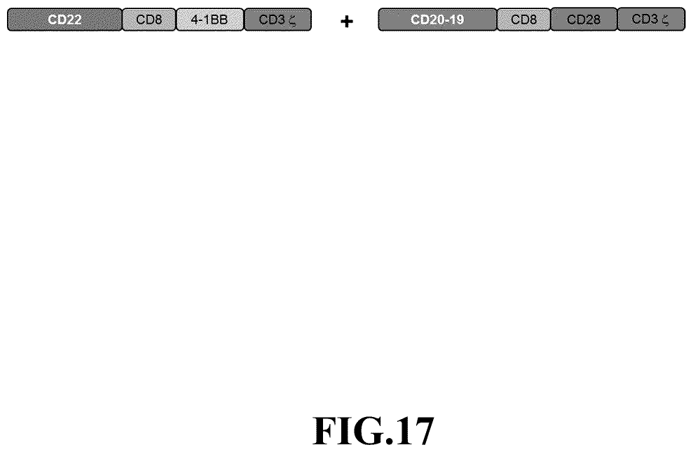

Having achieved the successful development of bicistronic DuoCARs targeting three distinct tumor antigens CD19, CD20, CD22 and comprised of two CAR chains possessing costimulatory domains with distinct and complimentary functions, the question was asked whether similar construct can be generated by other approaches. Successful bicistronic expression of separate CAR chains within the same ORF requires multiple optimization and refinement steps, and will be unique for each new set of sequences. By contrast, combining two CAR sequences during lentiviral vector manufacturing or during CAR T transduction, may offer a more universal approach and a fast method for creating CAR combinations to be expressed in the same cell, or same T cell population, while using a single lentiviral preparation for T cell transduction. In this example, as in the DuoCAR approach, one CAR chain is comprised of CD22 scFv, linked in frame to CD8 hinge and transmembrane domain, 4-1BB costimulatory domain and CD3 zeta activation domain. The second CAR chain is comprised of a tandem CD20 CD19 scFv-based targeting domain, followed by CD8 hinge and transmembrane domain, CD28 costimulatory domain and CD3 zeta activation domain (FIG. 17). In co-transfection approach, two transfer plasmids, each encoding one CAR chain, are mixed together and combined with the helper plasmids during vector production step, as per standard protocol (see materials and methods). The resulting lentiviral preparation will thus encode the mixture of the two CAR chains. Using this approach, a set of lentiviral preparations encoding two CAR chains simultaneously were generated (Table 2 infra).

TABLE 2
Constructs used in Co-Transfection co-transduction experiments

| CAR construct number | Description |
|---|---|
| D1 | MSCV-AscI-16P17- CD8 4-1BBz |
| D2 | MSCV-AscI-16P8- CD8 4-1BBz |
| D3 | MSCV-AscI-16P13- CD8 4-1BBz |
| 2273 | MSCV-20-19-28z |
| D1 + 2273 | combination |
| D2 + 2273 | combination |
| D3 + 2273 | combination |

Transfer plasmids for CAR 22 utilizing scFv 16P17, 16P8, 16P13 CAR22-4-1BB-CD3zeta, under the control of MSCV promoter (D1, D2, D3 respectively) and tandem CAR 2019-28-CD3zeta under the control of MSCV (LTG 2273) were constructed. Lentiviral vectors encoding each CAR chain alone were produced in parallel. High titers for all DuoCAR co-transfection preparations (10¹⁰ TU/ml, not shown) were routinely achieved, underscoring the efficiency of this approach.

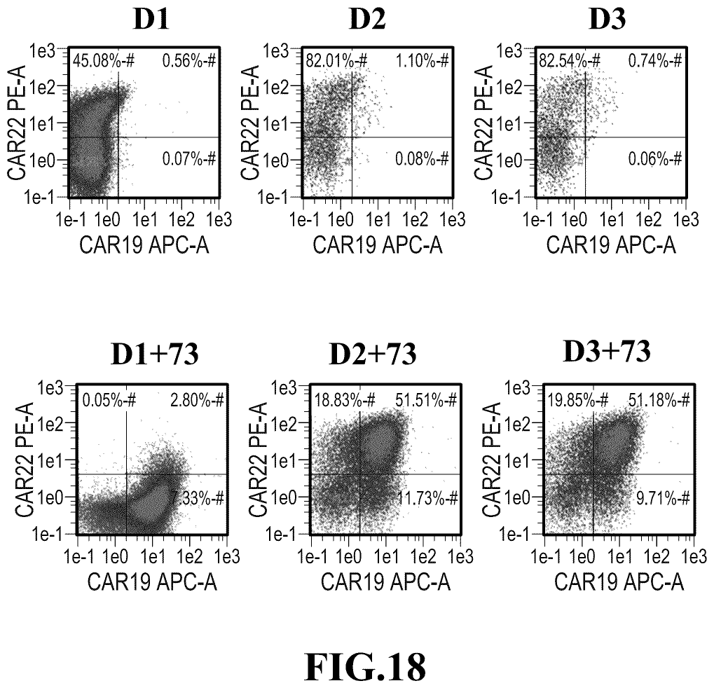

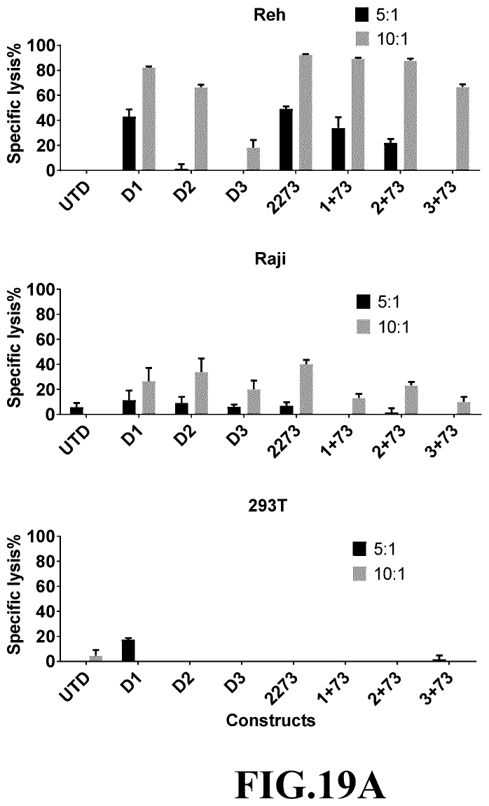

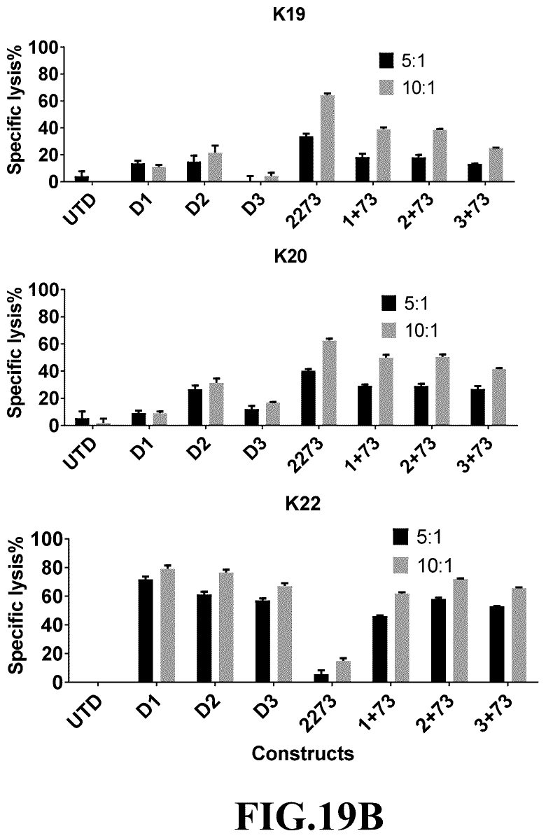

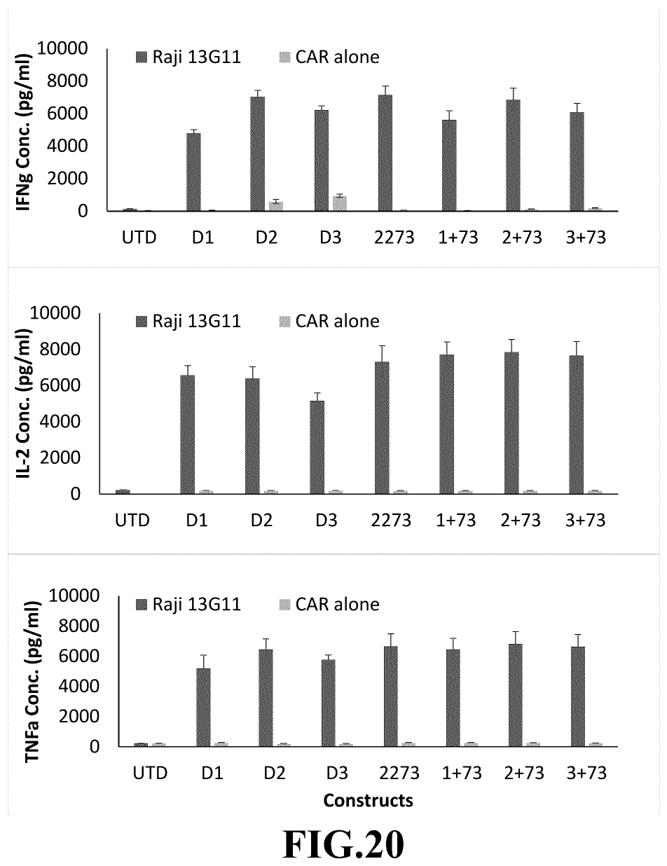

To optimize DuoCAR function, a series of CAR22 constructs comprised of scFvs 16P17, 16P8, and 16P13, were designed (constructs D1, D2, D3, respectively) under the control of MSCV promoter and used a tandem CAR 2019 (LTG2273), also driven by MSCV promoter for DuoCAR co-transfection combinations (Table 2 and FIG. 18). Lentiviral vectors were prepared by co-transfection of LTG2273 with one of the CD22 CAR plasmids, and yielded high infective titers (not shown). Each LV was used at multiplicity of infection (MOI) 20 for transduction of health donor T cells and CAR expression was determined by flow cytometry (FIG. 18). All control groups transduced with LV encoding a single CAR control yielded high CAR expression (45% for D1, 82% for D2, 82% for D3, 87% for 2273 (not shown). Surprisingly and unexpectedly, in combination co-transfection, groups D2+73 and D3+73 yielded efficient and nearly identical co-expression of the two CAR chains (51%), whereas combinations D1+73 failed to co-express (2.8% CAR+), FIG. 18. To determine whether these DuoCARs possess lytic function, CAR T cells from each group on a panel of tumor lines were tested (FIG. 19, in the labels of groups D1+2273, D2+2273, D3+2273, “D” was omitted for brevity). All DuoCAR preparations efficiently lysed triple-positive tumor lines Raji and Reh, but not triple negative line 293T, attesting to DuoCAR specificity (FIG. 19A). In addition, all DuoCARs showed above-background lytic function against single-antigen tumor lines K19, K20 and K22, whereas single control CARs with mismatched targeting domains showed no specific lysis: see D1 through D3 in K19; DI through D3 in K20, 2273 in K22, (FIG. 19B). The capability of DuoCARs to induce cytokines upon co-incubation with specific tumor targets was then assayed (FIG. 20; in the labels of groups D1+2273, D2+2273, D3+2273, “D” was omitted for brevity). DuoCAR T cell, single CAR controls and untransduced T cells (UTD) were combined with triple CD19+CD20+CD22+ Raji tumor cells and incubated overnight. In parallel, CAR T cells in the absence of tumor were incubated under similar conditions to rule out spontaneous cytokine release. At the end of incubation period, culture supernatants were assayed for cytokines IFNg, TNFa and IL-2 by ELISA (FIG. 20). All CAR groups produced high IFNg levels upon co-incubation with Raji. Whereas some single CD22 CAR controls had moderate spontaneous IFNg release (D2, D3), none of the DuoCARs produced IFNg spontaneously, suggesting a potential greater margin of safety for DuoCARs. IL-2 and TNFa expression were also highly induced by Raji co-incubation in all CAR groups with the exception of CAR 2272 (FIG. 20).

In summary, described here are the generation of functional and highly specific DuoCARs by co-transfection of individual CAR chains during LV preparation and applying the resulting LV preparation in T cell transduction. Moreover, using transgenic cell lines expressing only a single target antigen (K19, K20, K22) it was demonstrated that each of the CAR targeting domains is functional and can elicit DuoCAR function against target-expressing tumor cells. Surprisingly and unexpectedly, only a few combinations were able to demonstrate both robust CAR expression and potent cytotoxic function, therefore DuoCAR design is not trivial.

### Example 5

### Bicistronic DuoCARs Potently Eradicate Lymphoma Tumors.

### Materials and Methods Used in Example 5

### (a) Cell Lines

The Burkitt lymphoma cell line Raji, and the chronic myelogenous leukemia line K562 were purchased from American Tissue Culture Collection (ATCC, Manassas, VA). The REH leukemia line was purchased from DSMZ (Leibniz Institute DSMZ, Braunschwieg, Germany). Cells were cultured in RPMI-1640 medium supplemented with 10% heat-inactivated fetal bovine serum (FBS, Hyclone, Logan, UT) and 2 mM L-Glutamax (Thermo Fisher Scientific, Grand Island, NY). Human Embryonic kidney line 293T was purchased from ATCC (Gibco/Thermo Fisher Scientific, Grand Island, NY). Single-cell clones of luciferase-expressing cell lines were generated by stably transducing wild-type tumor lines with lentiviral vector encoding firefly luciferase (Lentigen Technology, Inc., Gaithersburg, MD), followed by cloning and selection of luciferase-positive clones. The Raji 13G11 clone was generated by passaging luciferase-transduced Raji cells in the mice and was selected for its proliferative capacity. Whole blood was collected from healthy volunteers at Oklahoma Blood Institute (OBI) with donors' written consent. Processed buffy coats were purchased from OBI (Oklahoma City, OK). The CD4-positive and CD8-positive human T cells were purified from buffy coats via positive selection using a 1:1 mixture of CD4- and CD8-MicroBeads (Miltenyi Biotec, Bergisch Gladbach, Germany) according to manufacturer's protocol.

### (b) Creation of Chimeric Antigen Receptor (CAR)—Expression Vectors

The DuoCAR constructs were designed as bicistronic sequences incorporation one tandem CD19- and CD20-targeting CAR, and one single CD22-targeting CAR. The bicistronic expression of the two CAR constructs from same mRNA template was facilitated by ribosomal skip element 2A. CAR antigen-binding single and tandem domains were derived from human anti-CD22 single chain variable fragments (ScFv), or the tandem 20-19 targeting scFv described previously (Schneider, D. et al., (2017). Journal for immunotherapy of cancer, 5 (1), 42.). The CAR T-encoding sequences were generated by linking the binder sequence in frame to CD8a linking and transmembrane domains (aa 123-191, Ref sequence ID NP_001759.3). The C-terminal segment of the CAR constructs contained a CD3 zeta signaling domain (CD247, aa 52-163, Ref sequence ID: NP_000725.1). Some designs also included a co-stimulatory domain, derived from human 4-1BB, ICOS, OX40 or CD27 proteins. CAR constructs sequences were cloned into a third generation lentiviral plasmid backbone (Lentigen Technology Inc., Gaithersburg, MD). Lentiviral vector (LV) containing supernatants were generated by transient transfection of HEK 293T cells and vector pelleted by centrifugation of lentiviral vector-containing supernatants, and stored at −80° C.

### (c) Primary T Cell Purification and Transduction

Human primary T cells from healthy volunteers were purified from whole blood or buffy coats (purchased from commercial provider with donor's written consent) using immunomagnetic bead selection of CD4+ and CD8+ cells according to manufacturer's protocol (Miltenyi Biotec, Bergisch Gladbach, Germany). T cells were cultivated in TexMACS medium supplemented with 200 IU/ml IL-2 at a density of 0.3 to 2×10⁶ cells/ml, activated with CD3/CD28 MACS® GMP T Cell TransAct reagent (Miltenyi Biotec) and transduced on day 2 with lentiviral vectors encoding CAR constructs in the presence of 10 ug/ml protamine sulfate (Sigma-Aldrich, St. Louis, MO) overnight, and media exchanged on day 3. Cultures were propagated in TexMACS medium supplemented with 200 IU/ml IL-2 until harvest on day 8-10.

### (d) Immune Effector Assays (CTL and Cytokine)

To determine cell-mediated cytotoxicity (CTL assay), 5,000 target cells stably transduced with firefly luciferase were combined with CAR T cells at various effector to target ratios and incubated overnight. SteadyGlo reagent (Promega, Madison WI) was added to each well and the resulting luminescence quantified as counts per second (sample CPS). Target only wells (max CPS) and target only wells plus 1% Tween-20 (min CPS) were used to determine assay range. Percent specific lysis was calculated as: (1−(sample CPS−min CPS)/(max CPS−min CPS)). Supernatants from co-cultures at E:T ratio of 10:1 were removed and analyzed by ELISA (eBioscience, San Diego, CA) for IFNγ, TNFα and IL-2 concentration.

### (e) Flow Cytometric Analysis

For cell staining, half a million CAR T transduced cells were harvested from culture, washed two times in cold AutoMACS buffer supplemented with 0.5% bovine serum albumin (Miltenyi Biotec), and CAR surface expression detected by staining with CD19-Fc and CD20-Biotin or CD19-Fc and CD22-His peptide followed by secondary peptide-specific fluorescent conjugates (Jackson ImmunoResearch, West Grove, PA). Anti-CD4 antibody conjugated to VioBlue fluorophore (Miltenyi Biotec) was used where indicated, as per vendors' protocol. Non-transduced cells were used as negative controls. Dead cells in all studies were excluded by 7AAD staining (BD Biosciences, San Jose, CA). Cells were washed twice and resuspended in 200 ul Staining Buffer before quantitative analysis by flow cytometry. Flow cytometric analysis was performed on a MACSQuant®10 Analyzer (Miltenyi Biotec), and data plots were generated using FlowJo software (Ashland, OR).

### Generation of Trivalent DuoCARs Targeting CD19, CD20, CD22

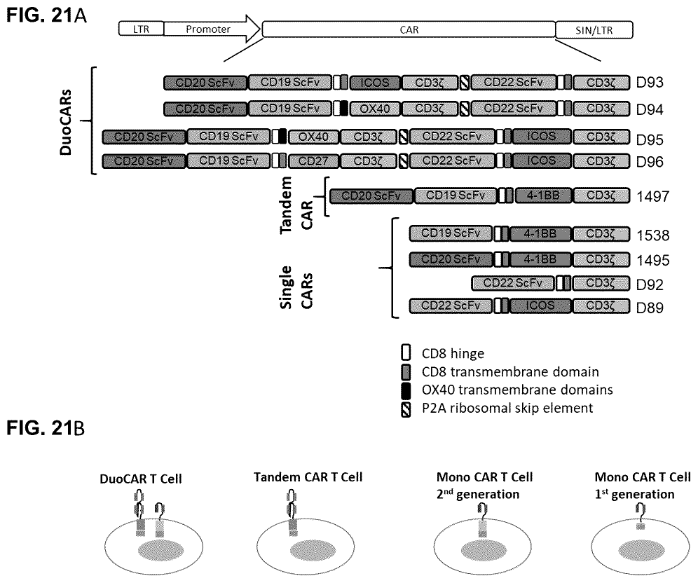

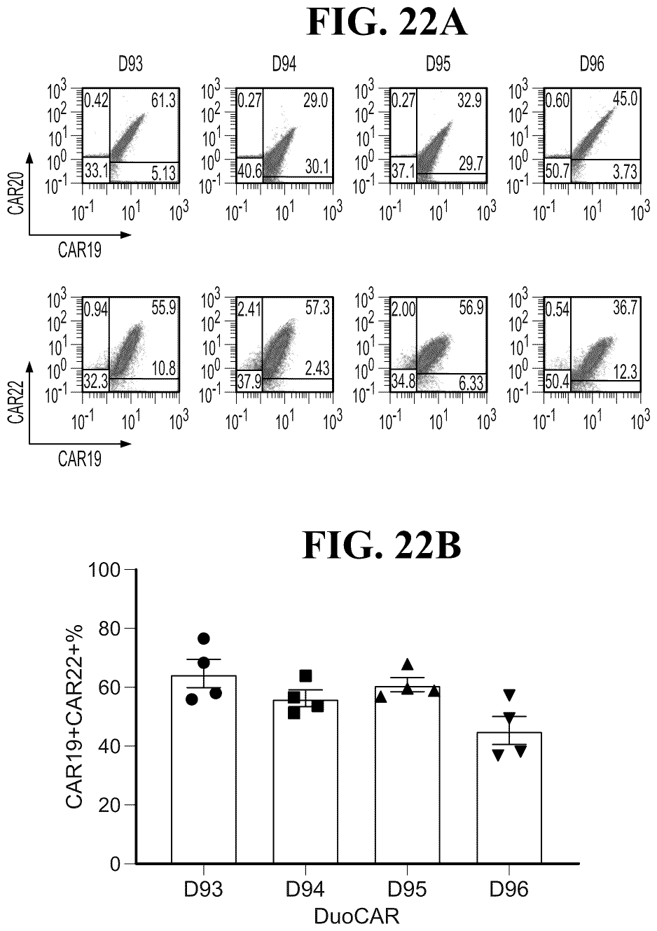

Trivalent DuoCARs were constructed by linking a tandem 2019-targeting CAR chain to a 22-targeting CAR chain via P2A ribosomal skip element. Four distinct DuoCAR constructs were designed based on best combinations previously identified in optimization co-transduction experiments. The optimization studies involved testing each CAR moiety (i.e. a CD20/19 tandem CAR and a CD22 single CAR) containing a 41BB, CD28, OX40, ICOS, or CD27 costimulatory domain, or no costimulatory domain, either alone or in combination. Specific parameters tested were CAR expression levels and in vitro anti-tumor activity. DuoCAR construct structures are shown in FIG. 21A, termed D93, D94, D95 and D96. DuoCAR Construct D93 was comprised of tandem scFv binder domain targeting B cell antigens CD19 and CD20, hinge and transmembrane domain derived from CD8, followed by ICOS co-stimulatory domain, and CD3ζ activation domains. This CAR construct sequence was linked via P2A ribosomal skip element to CD22-targeting first generation CAR, thus creating a bicistronic, triple-targeting DuoCAR (FIG. 21). Use of the 2A ribosomal skip element assures the following CAR attributes: (i) the production of one uniform cellular product, and (ii) stoichiometrically equal expression of the two CAR moieties within the individual cell; the combination of which achieves optimal anti-tumor function. DuoCAR construct D94 was identical to D93, except that the ICOS co-stimulatory domain was substituted for OX40 domain. Construct D95 was comprised of CD20- and CD19-tandem targeting OX40z CAR chain, identical to that of D94, followed by a second generation CD22 CAR chain with ICOS co-stimulatory domain and CD3ζ activation domain. Finally, DuoCAR construct D96 contained the CD20 and CD19 tandem targeting CAR chain with CD27 co-stimulatory domain, followed by CD22 targeting single CAR chain with ICOS co-stimulatory domain, each with CD3ζ activation domain (FIG. 21A). DuoCAR constructs were encoded into lentiviral vectors and transduced into human primary T cells. The DuoCARs were robustly expressed in T cells, ranging 30%-70% CAR T+ cells between three different constructs and donors (FIG. 22A, 22B).

In addition, several control CAR constructs, including single-targeting CARs and a tandem targeting CAR were constructed (FIG. 21A). The single-targeting CARs comprised either anti-CD22 scFv, anti-CD19 scFv, or anti-CD20 scFv, followed by CD8 hinge and transmembrane domain, either linked directly to the CD3z activation domain (D92, CAR22, first generation) or also incorporating a co-stimulatory domain (D89, 1538, 1495, second generation), FIG. 21A. The tandem control CAR targeting CD20 and CD19, termed 1497 CAR was comprised of the tandem CD20-CD19 scFv linked in frame to CD8 hinge and transmembrane domain, 4-1BB co stimulatory and CD3z activation domain. Tandem control constructs D88, D90, D91 were designed as tandem CAR 1497, except for the substitution of the 4-1BB co-stimulatory domain with ICOS, OX40 or CD27 domains, respectively (not shown). These constructs exhibit anti-tumor activity against unmodified tumors expressing all three target antigens, CD19, CD20 and CD22, but in contrast to the DuoCARs D93, D94, D95 and D96, the tandem constructs are not able to prevent antigen escape of tumor cells that are double-negative for CD19 and CD20. The positioning and composition of CAR chains within T cell is schematically shown for DuoCAR, tandem CAR, and single CAR T cell of the first or the second generation in FIG. 21B. All single and tandem CAR constructs achieved robust expression in human primary T cells by lentiviral transduction.

### DuoCARs Potently and Specifically Lyse Tumor Targets In Vitro

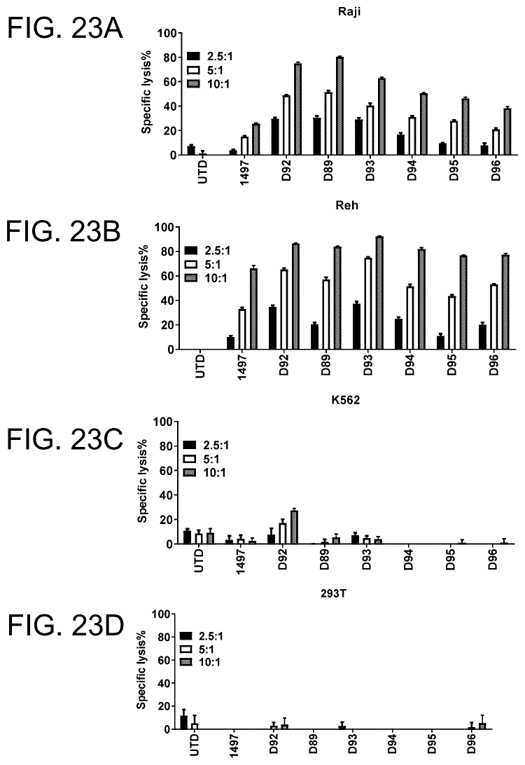

In order to evaluate the functionality of the constructed DuoCARs, the constructed DuoCARs were combined with luciferase-expressing target tumor lines for an overnight killing assay. Antigen-positive lines, the NHL line Raji (CD19+CD20+CD22+), and the B-ALL line Reh (CD19+CD20lowCD22+) were used to test the capability of DuoCARs to lyse tumor cells in antigen-specific manner. Negative control lines, myelogenous leukemia K562, and human embryonic kidney line 293T, which are both CD19−CD20−CD22−, were also included (FIG. 23).

Tandem CAR 1497, and single CAR controls D89 and D92, the CD22 targeting CARs of the second and the first generation, respectively, were included for comparison.

CAR T cells and target cells were combined at effector to target (E:T) ratios of 10:1, 5:1 or 2.5:1, and at the completion of incubation period specific lysis was calculated for each condition as described in Materials and methods.

All single and tandem CARs lysed target-positive tumor lines, Raji and Reh, in E:T-dependent manner (FIG. 23A, 23B). The DuoCARs constructs potently lysed target cell lines which express the targeted antigens CD19, CD20 and CD22 at all E:T ratios (FIG. 23A, 23B). The tandem control CAR 1497, targeting the CD19 and CD20 antigens, resulted in a relatively modest tumor lysis at the E:T ratio tested, as compared to the DuoCARs D93, D94, D95 and D96 in Raji cells (FIG. 23A). By comparison, in Reh cells, the lytic potency of 1497 was similar to the lytic potency of DuoCARs D93, D94, D95 and D96 (FIG. 23B). The single-targeting CAR22 constructs D89 and D92 tended to be the most potent tumor cell killers for the CD22 antigen-positive target lines Raji and Reh. By contrast, none of the DuoCAR constructs or controls lysed target-negative tumor lines K562 and 293T (FIG. 23C, 23D), with the exception of single targeting CAR22 construct D92 in K562 cells, which produced 27% non-specific lysis at the highest E:T ratio of 10 (FIG. 23C). Therefore, the DuoCARs performed equally well, or were superior to 1497 tandem construct in the lysis of antigen-positive target lines, and produced no background lysis in antigen negative lines, demonstrating antigen-dependence. Notably, despite non-specific lytic activity of the single CAR 22, D92, in K562 cells, the incorporation D92 sequence into DuoCAR constructs D1 and D2 did not result in non-specific target lysis by DuoCARs. Therefore, the DuoCAR design appears to tamper down the undesired spontaneous lytic activity seen in the first generation CAR D92 (FIG. 23C).

### Cytokine Response of DuoCARs

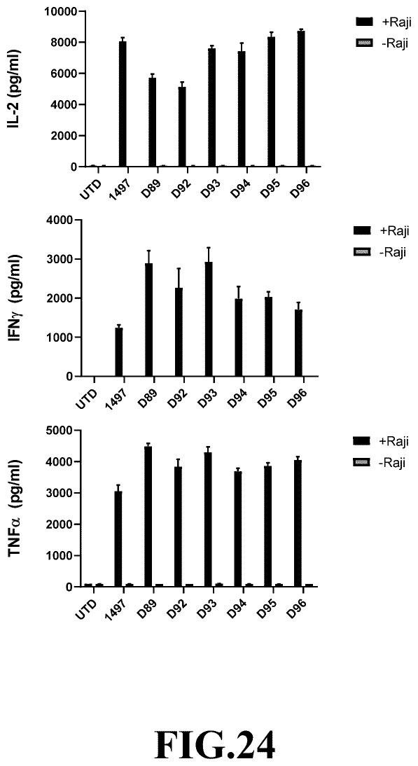

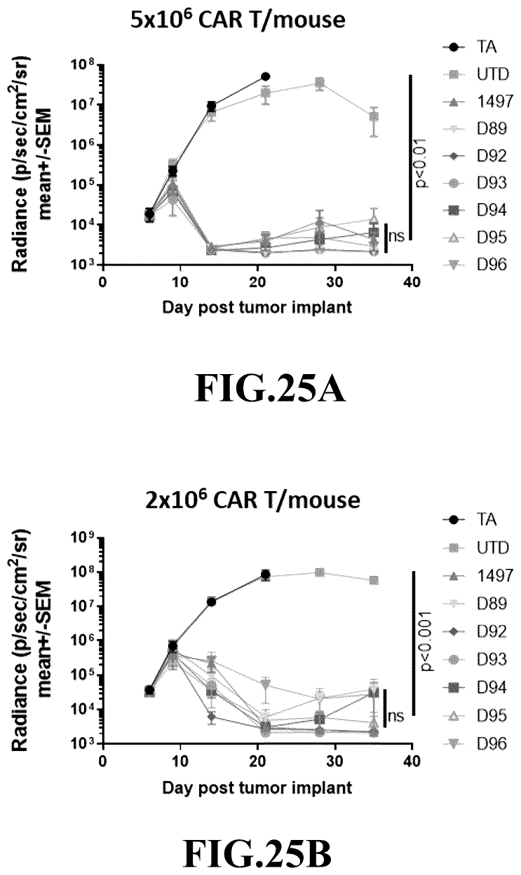

To characterize the cytokine production of DuoCARs in response to target cells, supernatants were collected form co-cultures of DuoCARs with CD19+CD20+CD22+ Raji target cells following overnight incubation. The concentration of T cell pro-inflammatory and homeostatic cytokines IL-2, IFNγ and TNFα in culture supernatants were determined by ELISA (FIG. 24, blue bars). Non-transduced T cells from same donor and batch (UTD) were included as a CAR-negative control. Further, to control for possible spontaneous cytokine release by CAR T cells in the absence of triggering target cell, each CAR T cell group was also incubated without Raji targets (FIG. 24, light grey bars). DuoCARs, as well as single and tandem control CARs strongly induced the production of IL-2, IFNγ and TNFα in response to target Raji cells as compared to UTD, however none of the DuoCAR lines or CAR controls were prone to spontaneous release of these soluble factors in the absence of target cells (FIG. 24).

### DuoCAR Efficiently Lyse CD19+CD20+CD22+ Raji Tumors In Vivo

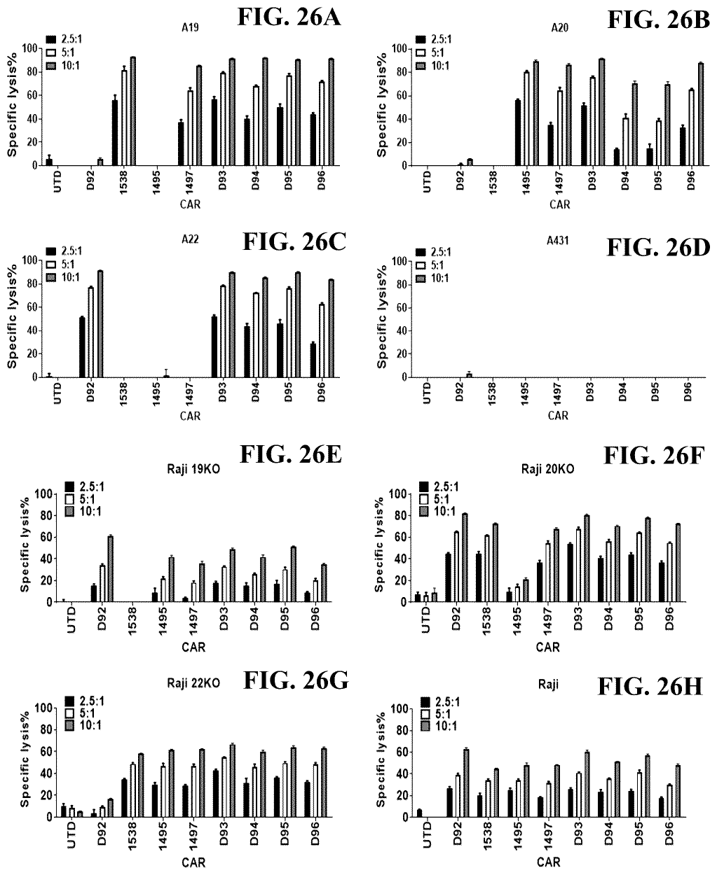

Having established the cytotoxic and cytokine release functionality of DuoCARs against antigen-positive target cells in vitro, DuoCAR function was then demonstrated in vivo. NSG (NOD.Cg-Prkdc^{scid}Il2rg^{tm1Wjl}/SzJ) mouse xenograft of Raji cells stably transduced with firefly luciferase was utilized. DuoCARs D93, D94. D95 and D96, as well as tandem control CAR 1497, and single controls D89 and D92 were included (FIG. 26A). Tumor bearing mice were administered either five million CAR T cells, or two million CAR T cells each, or dose-matched UTD controls, on study day 7, and tumor rejection was measured by bioluminescent imaging periodically up to study day 28 (FIGS. 26B-26E). In the high CAR T dose regiment of five million cells per mouse, DuoCARs potently suppressed Raji tumor progression from day 14 and onward, whereas tumors in tumor alone group (TA), and the non-transduced T cell group (UTD) progressed unabated. The tumor repression mediated by DuoCARs and single and tandem CAR controls, as compared to the TA and UTD negative controls, was statistically significant. The DuoCAR D93, and the single CARs D89 and D92 tended to generate the greatest tumor regression over the study period, whereas the DuoCAR D95 and the tandem CAR 1497 tended to be the least potent among the CAR constructs. However, the differences between the individual CAR constructs at this dosage level were not statistically significant (FIG. 26B). To better pinpoint the minor differences between DuoCAR constructs and to test whether they remain functional at low dose regiments, Raji-bearing mice were dosed with two million CAR T cells each (FIG. 26C, 26E). Despite the low CAR T dose, all CAR constructs significantly controlled Raji tumor burden as compared to TA and UTD controls at this level. Whereas none of the DuoCAR constructs or control CARs were significantly better than other CARs, DuoCAR D93 and D94 tended to maintain best tumor control, whereas DuoCAR D96 tended to be less potent than other CARs (FIG. 26C). Of note, tumor regression in 1497, the tandem CAR group, appeared delayed as compared to DuoCARs D93-D96, suggesting a possible superiority of the DuoCAR constructs in this setting (FIG. 26C). In addition, The single CAR D92 tended to reduce tumor burden faster than the other CAR constructs, in concordance with high potency, but also lower specificity observed for this construct in the in vitro cytotoxicity experiment against antigen-positive and antigen-negative target lines (FIG. 23).

Trispecific DuoCARs require only a single antigen for anti-tumor function, and are potently killing antigen-erased target cells in a model of tumor antigen loss of either CD19, CD20, or CD22.

Having demonstrated that the DuoCARs mediated a potent rejection of CD19+CD20+CD22+, wild-type Raji xenograft in vivo, even at the low dose regiment of two million CAR T+ cells/mouse, the sufficiency of each single antigen to trigger DuoCAR activation was verified in vitro. CAR constructs included in this experiment are schematically shown in FIG. 26A. Experimental groups included the DuoCARs D93, D94, D95 and D96, the tandem CAR control 1497, and single CAR controls D92, 1538 and 1495, targeting the CD22, CD19 and the CD20 antigens, respectively (FIG. 26).

The DuoCARs are postulated to function as a logical [OR] gate constructs, such as the presence of either one or more of the three targeted antigens is sufficient for triggering CAR activation and anti-tumor function. It was then confirmed that each of the three reactivities is intact in DuoCARs D93, D93, D95, and D96. To this end, the A431 squamous cell carcinoma line was engineered, which is naturally devoid of B cell surface molecules, to stably express either CD19, or CD20, or CD22. To facilitate quantitation of tumor lysis, each target A431 line also stably expressed firefly luciferase. DuoCAR T cells and control CARs were tested in in vitro cytotoxicity assay against each of the A431 clones expression one antigen only, and the parental A431 parental line was included as a control target-negative (FIG. 26A-26D).

DuoCARs D93, D93, D95, and D96 effectively lysed tumors expressing a single antigen each only: CD19 (FIG. 26A), CD20 (FIG. 26B) or CD22 (FIG. 26C), but not the parental line A431 lacking the expression of these target molecules (FIG. 26D). Tumor lysis by DuoCARs of target cells with cognate antigen expression was dependent on effector to target ratio, demonstrating precise specificity of the DuoCAR constructs. As expected, single CARs could not lyse target clones if those clones lacked the expression of the targeted antigens. Line A19 was lysed by single CAR19, 1538, but not by single CAR targeting CD22-D92, or targeting CD20-1495 (FIG. 26A). Similarly, target line A20 was lysed by CAR20 1495, but not by single CARs CD22-D92, or CD19 CAR-1538 (FIG. 26B). Moreover only the CD22-targeted single CARs D92 and D89, but not the CD19 and CD20 targeted CARs 1538 and 1495, respectively, lysed the A22 target line (FIG. 26C). None of the constructs lysed the parental line A431, in concordance with lack of expression of CD19, CD20 or CD22 on this tumor line (FIG. 26D). Therefore, DuoCARs were reactive with each one of the target antigens CD19, CD20, CD22, in an isolated fashion, independently of other two antigens. Further, the presence of each single antigen CD19, CD20 or CD22 in isolation was sufficient to trigger DuoCAR function.

It was then shown that in a model of tumor antigen escape, Raji clones with erased expression of either CD19, CD20 or CD22, DuoCARs were able to lyse target cells despite the lack of either one of the three targeted molecules (FIG. 26E-26G), and the magnitude of lysis was comparable to the DuoCAR lysis of the parental Raji line, in which the expression of all three antigens was intact (FIG. 26H). By contrast, the single-targeting CARs were lytic only against clones in which their cognate targets were present. Specifically, single CARs 20 and 22, D92 and 1495, but not single CAR 19, 1538, lysed Raji 19KO (FIG. 26E), Single CARs 19 and CAR22, but not CAR 20 lysed the Raji 20KO (FIG. 26F), and single CAR 19 and 20, but not CAR22 lysed the Raji 22 KO line (FIG. 26G). By comparison, none of the single CAR controls D92, 1538 or 1495 showed any impairment in the lysis of the parental Raji clone, expressing all three target antigens (FIG. 26H). These results demonstrate the superiority of DuoCARs in targeting tumor cells which lack the expression of one of the targeted antigens.

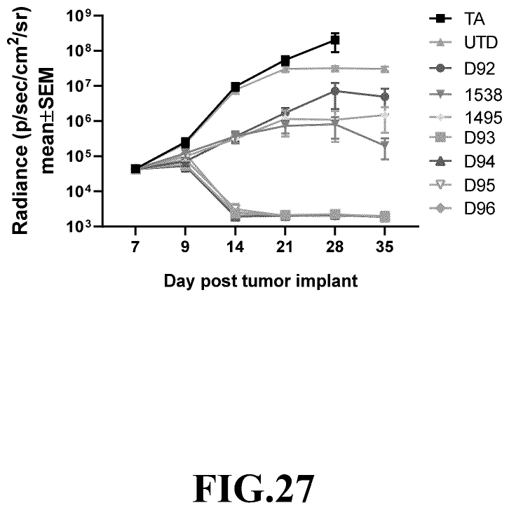

Tumor antigen escape, when expression of one or more of the targeted antigens diminishes or disappears completely, and tumor heterogeneity, whereas a single agent/CAR is incapable to target tumor cell population in its entirety, due to heterogeneous expression of the targeted antigen, remain a major obstacle to CAR T immunotherapy. To demonstrate the ability of DuoCARs to combat tumors which have lost expression of some of their target antigens, a heterogeneous xenograft tumor model was generated (FIG. 27). NSG mice were implanted with a mixture of luciferase-positive, antigen-deleted Raji clones: Raji 19KO, Raji 20KO, Raji 22KO, and the parental Raji clone, at equal proportions. Seven days after tumor implant, mice were treated with five million CAR T+ DuoCAR cells, or single CAR controls targeting CD19, CD20 or CD22. Tumor burden was measured by bioluminescence. Strikingly, starting on study day 14 an downwards, DuoCARs D93, D94, D95 or D96 have completely rejected the heterogeneous Raji tumors. By contrast, tumors continued to grow in mice receiving single targeting CARs CAR19-1538, CAR20-1495 or CAR22-D92 (FIG. 27).

In summary, described here are four novel DuoCAR designs, D93. D94, D95 and D96, which enable production of highly-functional, triple-targeting CAR T cells. DuoCAR T cells were reactive to CD19, CD20 and CD22 antigens in vitro and in vivo with high specificity, and demonstrated an extremely potent function and complete tumor rejection in a disseminated in vivo xenograft Raji tumor model with varied expression of CD19, CD20 and CD22, whereas single-targeting CARs could not prevent tumor progression in this model of tumor antigen escape. Therefore, DuoCAR T cells represent a novel solution to tackling antigen-heterogeneous tumor population and mitigating tumor antigen escape, and thus provide an opportunity for improving clinical outcomes in CAR T-treated patient population.

1.-22. (canceled)

23. A pharmaceutical composition comprising an antitumor effective amount of a population of human natural killer (NK) cells, wherein the NK cells of the population include NK cells comprising one or more multicistronic vectors; wherein each multicistronic vector encodes a functional chimeric antigen receptor (CAR) comprising the amino acid sequence of SEQ ID NO: 110, 112, 114, or 116.

24.-32. (canceled)

33. A method of treating a subject having a cancer, the method comprising administering to the subject a pharmaceutical composition comprising an antitumor effective amount of a population of human NK cells, wherein the population of human NK cells comprises NK cells comprising two or more vectors; wherein each vector encodes a functional CAR comprising the amino acid sequence of SEQ ID NO: 110, 112, 114, or 116.

34. (canceled)

35. The method of claim 33, wherein the genetically modified NK cells are autologous NK cells, and wherein the autologous or allogeneic NK cells are infused directly back into the patient so as to prevent malignant disease relapse.

36. The method of claim 33, wherein the genetically modified NK cells are autologous NK cells, and wherein the autologous NK cells are infused directly back into the patient to promote in vivo expansion of the CAR-NK cells, cancer stabilization, reduction of the cancer, or remission of the cancer in the subject.

37.-38. (canceled)

39. The method of claim 33, wherein the cancer is a hematological cancer.

40. The method of claim 39, wherein the hematological cancer is leukemia, lymphoma, or multiple myeloma.

41. The method of claim 39, wherein the leukemia is chronic lymphocytic leukemia (CLL), acute lymphocytic leukemia (ALL), acute myeloid leukemia (AML), or chronic myelogenous leukemia (CML).

42. The method of claim 39, wherein the lymphoma is mantle cell lymphoma, non-Hodgkin's lymphoma or Hodgkin's lymphoma.

43. The method of claim 33, wherein the cancer is selected from the group consisting of oral and pharynx cancer, a digestive system cancer, a respiratory cancer, a bone and joint cancer, a soft tissue cancer, a skin cancer, a pediatric cancer, a tumor of the central nervous system, a cancer of the breast, a cancer of the genital system, a cancer of the urinary system, a cancer of the eye and orbit, a cancer of the endocrine system, and a cancer of the brain and other nervous system.
